(19) United States
(12) Patent Application Publication
(10) Pub. No.: US 2026/0053798 A1
(43) Pub. Date: Feb. 26, 2026

(54) COMBINATION THERAPY FOR A RAS RELATED DISEASE OR DISORDER

(71) Applicant: Revolution Medicines, Inc., Redwood City, CA (US)

(72) Inventors: Ida ARONCHIK, Burlingame, CA (US); Cristina BLAJ, Oakland, CA (US); Lingyan JIANG, San Mateo, CA (US); Jingjing JIANG, Redwood City, CA (US); Mark LABRECQUE, Redwood City, CA (US); Bianca Jennifer LEE, Redwood City, CA (US); Marie MENARD, Foster City, CA (US); Elsa QUINTANA, Belmont, CA (US); Kyle SEAMON, San Mateo, CA (US); Lillian SEU, San Francisco, CA (US); Mallika SINGH, San Francisco, CA (US); Nataliya Tovbis SHIFRIN, San Carlos, CA (US); Vidyasiri VEMULAPALLI, San Mateo, CA (US); Yingyun WANG, San Francisco, CA (US); Xing WEI, San Carlos, CA (US); Caroline E. WELLER, Redwood City, CA (US); David E. WILDES, San Francisco, CA (US); Yu Chi YANG, Foster City, CA (US); Yongxian ZHUANG, Milpitas, CA (US); David Church MONTGOMERY, San Carlos, CA (US)

(21) Appl. No.: 19/377,386

(22) Filed: Nov. 3, 2025

Related U.S. Application Data
(63) Continuation of application No. PCT/US2024/027781, filed on May 3, 2024.
(60) Provisional application No. 63/464,023, filed on May 4, 2023.
(60) Provisional application No. 63/545,097, filed on Oct. 20, 2023.
(60) Provisional application No. 63/551,254, filed on Feb. 8, 2024.

(51) Int. Cl. A61K 31/504; A61K 31/282; A61K 31/337; A61K 31/475; A61K 31/506; A61K 31/513; A61K 31/5386; A61K 31/7068; A61K 39/395; A61P 35/00

(52) U.S. Cl. CPC A61K 31/504; A61K 31/282; A61K 31/337; A61K 31/475; A61K 31/506; A61K 31/513; A61K 31/5386; A61K 31/7068; A61K 39/39558; A61P 35/00

(57) ABSTRACT

The present disclosure relates to combination therapies for treating a RAS related disease or disorder (e.g., cancer). In particular, the present disclosure relates to methods of treating a RAS related disease or disorder in a subject in need thereof, comprising administering to the subject a therapeutically effective amount of a RAS(ON) inhibitor in combination with one or more additional therapeutic agents, pharmaceutical compositions comprising a therapeutically effective amounts of the same, kits comprising the compositions and methods of use therefor.

### FIELD

The present disclosure relates to combination therapies useful for treating aberrant RAS signaling (e.g., treating RAS addicted cancer). Provided herein, in certain aspects, are compositions comprising therapeutically effective combinations comprising a RAS(ON) inhibitor, pharmaceutical composition comprising the combination therapies and methods of using the same in the treatment of a diseases or disorders having modulated RAS activity.

### BACKGROUND

It has been well established in literature that RAS proteins (K-RAS, H-RAS, and N-RAS) play an essential role in various RAS related diseases, such as cancer, RASopathies, and neurological disorders, where mutations in the RAS genes or their regulators render RAS proteins persistently active and are therefore appropriate targets for therapy. Indeed, mutations in RAS proteins account for approximately 30% of all human cancers in the United States, many of which are fatal. Dysregulation of RAS proteins by activating mutations, overexpression or upstream activation is common in human tumors, and activating mutations in RAS are frequently found in human cancer. For example, activating mutations at codon 12 in RAS proteins function by inhibiting both GTPase-activating protein (GAP)-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codon 13 (e.g., G13D) and 61 (e.g., Q61K) of RAS are also responsible for oncogenic activity in some cancers.

Despite decades of extensive drug discovery efforts targeting aberrant RAS-signaling, the presence of acquired and adaptive resistance mechanisms associated with RAS mutant cancers make achieving a complete and durable response difficult. Additional efforts are needed to uncover additional compositions, including those with combinations of therapeutic agents, for targeting RAS-driven diseases and disorders.

### SUMMARY

The present disclosure provides compositions and uses thereof for treating a RAS related disease or disorder (e.g., cancer) comprising a RAS(ON) inhibitor and one or more additional therapeutic agents. This disclosure is based, at least in part, on the observation that a RAS related disease or disorder, such as cancer, can be treated with a combination of i) one or more RAS(ON) inhibitors and ii) one or more additional therapeutic agents. In various embodiments disclosed herein the RAS related disease or disorder (e.g., cancer) is resistant to monotherapy with a therapeutic agent disclosed herein.

In one aspect, the present disclosure provides a method of treating cancer in a subject in need thereof, the method generally comprises administering to the subject a therapeutically effective amount of a combination, comprising i) a RAS(ON) inhibitor; and ii) one or more additional therapeutic agents.

In another aspect, the present disclosure provides a method of treating a RAS related disease or disorder in a subject in need thereof, the method generally comprises administering to the subject a therapeutically effective amount of a combination, comprising i) a RAS(ON) inhibitor; and ii) one or more additional therapeutic agents.

In still another aspect, the present disclosure provides a method of modulating RAS activity and activity of one or more target proteins, in a cell, the method generally comprises administering to the cell an effective amount of a combination, comprising i) a RAS(ON) inhibitor; and ii) one or more additional therapeutic agents, wherein the one more additional therapeutic agents modulate the activity of the one or more target proteins.

In still yet another aspect, the present disclosure provides a method of inhibiting RAS activity and activity of one or more target proteins, in a cell, the method generally comprises administering to the cell an effective amount of a combination, comprising i) a RAS(ON) inhibitor; and ii) one or more additional therapeutic agents, wherein the one more additional therapeutic agents modulate the activity of the one or more target proteins.

In each of the above aspects, the additional therapeutic agent may be a second, distinct RAS(ON) inhibitor (e.g., a RAS(ON) mutant-selective inhibitor or a RAS(ON) multi-selective inhibitor). In each of the above aspects, the RAS(ON) inhibitor may be selected from a RAS(ON) inhibitor described herein, for example those disclosed in section I (A).

In some embodiments, the one or more additional therapeutic agents is a RAS/MAPK pathway inhibitor, a Kinase inhibitor, a Receptor Tyrosine Kinase inhibitor, a PI3K/mTOR pathway inhibitor, a DNA Damage Response inhibitor, a Cell Cycle inhibitor, an Anti-apoptotic protein inhibitor, an Autophagy inhibitor, a Macropinocytosis inhibitor, a Wnt/Beta-catenin pathway inhibitor, a JAK/STAT pathway inhibitor, an Epigenetic modulator, an immunotherapy, a farnesyl transferase inhibitor, a TGFbeta inhibitor, a HSP90 inhibitor, a GPX4 inhibitor, a NRF2 inhibitor, a TEAD inhibitor, a NOTCH inhibitor, a gamma secretase inhibitor, a hedgehog inhibitor, a chemotherapeutic, a proteasome inhibitor, or any combination thereof.

In some embodiments, the RAS/MAPK pathway inhibitor is a RAS (OFF) inhibitor, a SOS1 inhibitor, a SHP2 inhibitor, a MEK inhibitor, a RAF inhibitor, a ERK inhibitor, a MAPK inhibitor, or any combination thereof.

In some embodiments, the RAS (OFF) inhibitor is selected from a RAS (OFF) inhibitor described herein, for example, those disclosed in section I (b) (i).

In some embodiments, the SOS1 inhibitor is RMC-5845, RMC-4948, RMC-0331, BI-1701963, BI-3406, SDR5, MRTX0902, BAY-293, or any combination thereof.

In some embodiments, the SHP2 inhibitor is SHP099, TNO155, RMC-4550, RMC-4630, JAB-3068, JAB-3312, RLY-1971, ERAS-601, SH3809, PF-07284892, BBP-398, or any combination thereof. In some embodiments, the MEK inhibitor is pimasertib, selumetinib, cobimetinib, trametinib, binimetinib, or any combination thereof.

In some embodiments, the RAF inhibitor is VS-6766, IK-595, vemurafenib, dabrafenib, and encorafenib, or any combination thereof.

In some embodiments, the ERK inhibitor is ASTX-029, 1-75, or a combination thereof.

In some embodiments, the MAPK inhibitor is Tilpisertib (GS-4875), neflamapidmod (VX-745), or a combination thereof.

In some embodiments, the kinase inhibitor is a PKA inhibitor, a FAK inhibitor, a ROCK inhibitor, a MSK1 inhibitor, a RSK inhibitor, an ALK inhibitor, or any combination thereof. In some embodiments, the PKA inhibitor is H89. In some embodiments, the FAK inhibitor is BI853520, defactinib, GSK2256098, PF-00562271, VS-4718, or any combination thereof. In some embodiments, the ROCK inhibitor is GSK269962A. In some embodiments, the MSK1 inhibitor is SB-747651A, SB 747651A, Ro 320432, CGP 57380, GSK2830371, SR1664, LY-3214996, PFI-4, MSC-2363318A, AS601245, or any combination thereof. In some embodiments, the RSK inhibitor is BI-D1870, LJH685, SL0101-1, FMK, BRD7389, BIX 02565, LJI308, LJI308-S, LJI308-1, LJH685-S, or any combination thereof. In some embodiments, the ALK inhibitor is Crizotinib, Ceritinib, Alectinib, Brigatinib, Lorlatinib, Ensartinib (X-396), TAE684, ASP3026, TPX-0131, LDK378 (Ceritinib analog), CEP-37440; 4SC-203, TL-398, PLB1003, TSR-011, CT-707, TPX-0005, AP26113, or any combination thereof.

In some embodiments, the Receptor Tyrosine Kinase inhibitor is an EGFR inhibitor, a HER2 inhibitor, a MET inhibitor, an AXL inhibitor, an IGFR inhibitor, a RET inhibitor, a ROS1 inhibitor, a PDGFR inhibitor, a FGFR inhibitor, a VEGF inhibitor, or any combination thereof.

In some embodiments, the EGFR inhibitor is osimertinib, cetuximab, gefitinib (Iressa), erlotinib (Tarceva), lazertinib, afatinib (Gilotrif), or any combination thereof. In some embodiments, the HER2 inhibitor is tucatinib. In some embodiments, the MET inhibitor is Crizotinib (Xalkori), Cabozantinib (Cometriq, Cabometyx), Capmatinib (Tabrecta), Tepotinib (Tepmetko), Savolitinib (Volitinib), Onartuzumab (MetMab), Foretinib (GSK1363089), MGCD-265 (Amuvatinib), SU11274, SU5416, or any combination thereof. In some embodiments, the AXL inhibitor is bemcentib, BGB324, R428, SGI-7079, TP-0903, BMS-777607, UNC2025, TP-0903, or any combination thereof. In some embodiments, the IGFR inhibitor is linsitinib, AXL1717, OSI-906 (Linsitinib), BMS-754807, BI 836845, AZ12253801, PQIP (Pyrrolo[1,2-a]quinoxaline), NVP-AEW541, or any combination thereof. In some embodiments, the RET inhibitor is pralsetinib, selpercatinib (LOXO-292), BLU-667, RXDX-105, TPX-0046, GSK3179106, molidustat (BAY 85-3934), RPI-1 (Retrophin), or any combination thereof. In some embodiments, the ROS1 inhibitor is taletrectinib, DS-6051b, TPX-0131, GZD824, PF-06463922, or any combination thereof. In some embodiments, the PDGFR inhibitor is CP-673451, imatinib, nintedanib (ofev), sunitinib (sutent), pazopanib (votrient), regorafenib (stivarga), dasatinib (sprycel), or any combination thereof. In some embodiments, the FGFR is futibatinib (TAK-659), erdafitinib (balversa), infigratinib (Truseltiq), Debio 1347, rogaratinib (BAY 1163877), or any combination thereof. In some embodiments, the VEGF inhibitor is bevacizumab, aflibercept, ramucirumab, sorafenib, sunitinib, pazopanib, or any combination thereof.

In some embodiments, the PI3K/mTOR pathway inhibitor is a PI3K inhibitor, an AKT inhibitor, an mTOR inhibitor, an MNK inhibitor, an eIF4 inhibitor, or any combination thereof.

In some embodiments, the PI3K inhibitor is alpelisib, copanlisib, or a combination thereof. In some embodiments, the AKT inhibitor is ipatasertib, GSK-2141795, Akt-1-1, Akt-1-1,2, a 1-H-imidazo[4,5-c]pyridinyl derivative, indole-3-carbinol or a derivative thereof, perifosine, a phosphatidylinositol ether lipid analog, triciribine, or any combination thereof. In some embodiments, the mTOR inhibitor is RMC-5552, PI-103, PP242, PP30, Torin 1, an FKBP12 enhancer, a 4H-1-benzopyran-4-one derivative, rapamycin (sirolimus), a rapalog, temsirolimus, everolimus, ridaforolimus, AP23464, AP23841, 40-(2-hydroxyethyl) rapamycin, 40-[3-hydroxy (hydroxymethyl)methylpropanoate]-rapamycin (CC1779), 40-epi-(tetrazolyt)-rapamycin (ABT578), 32-deoxorapamycin, 16-pentynyloxy-32 (S)-dihydrorapanycin, a phosphorus-containing rapamycin derivative, or any combination thereof. In some embodiments, the MNK inhibitor is tomivosertib (eFT508), CGP57380, and SEL201, or any combination thereof. In some embodiments, the eIF4 inhibitor is an eIF4A inhibitor or an eIF4G inhibitor. In some embodiments, the eIF4A inhibitor is zotatifin (eFT226), silvestrol, pateamine A, a rocaglate, or any combination thereof. In some embodiments, the eIF4G inhibitor is pateamine A, hippuristanol, or any combination thereof.

In some embodiments, the DNA damage response inhibitor is a Wee1 inhibitor, a CHK inhibitor, an ATM inhibitor, an ATR inhibitor, a PARP inhibitor, a DNA-PK inhibitor, or any combination thereof. In some embodiments, the Wee1 inhibitor is adavosertib, AZD1775, ZNL-02-096, MK-1775, or any combination thereof. In some embodiments, the CHK inhibitor is a CHK1 or a CHK2 inhibitor. In some embodiments, the CHK inhibitor is rabusertib, LY2606368, GDC-0575, MK-8776, or any combination thereof. In some embodiments, the ATM inhibitor is M4076, AZD0156, KU-60019, VE-821, or any combination thereof. In some embodiments, the ATR inhibitor is ceralasertib, VX-970, AZD6738, BAY 1895344, or any combination thereof. In some embodiments, the PARP inhibitor is olaparib, rucaparib, niraparib, veliparib (ABT-888), or any combination thereof. In some embodiments, the DNA-PK inhibitor is NU7441, AZD7648, VX-984, M3814, CC-115, SCR7, or any combination thereof.

In some embodiments, the cell cycle inhibitor is a CDK inhibitor, an Aurora kinase inhibitor, a PLK inhibitor, a KSP inhibitor, or any combination thereof. In some embodiments, the CDK inhibitor is a CDK2 inhibitor, a CDK4/6 inhibitor, a CDK7 inhibitor, or a CDK9 inhibitor, or any combination thereof. In some embodiments, the CDK inhibitor is seliciclib, UCN-01, P1446A-05, PD-0332991, dinaciclib, P27-00, AT-7519, RGB286638, SCH727965, AZD4573, or any combination thereof. In some embodiments, the Aurora kinase inhibitor is palbociclib, ribociclib, abemaciclib, alisertib, danusertib, barasertib, MLN8237, or any combination thereof. In some embodiments, the PLK inhibitor is volasertib, onvansertib, BI 2536, GSK461364, or any combination thereof. In some embodiments, the KSP inhibitor is SB743921, monastrol, S-Trityl-L-cysteine (STLC), filanesib (ARRY-520), AMG650, BTB-1, K03861, SJ000291942, or any combination thereof. In some embodiments, the anti-apoptotic inhibitors is a Bcl inhibitor, an XIAP inhibitor, a survivin inhibitor, an Mcl-1 inhibitor, or a FLIP inhibitor, or any combination thereof. In some embodiments, the Bcl inhibitor is ABT-263, Venetoclax (Venclexta), Navitoclax (ABT-263), A-1331852, S63845, AT-101, or any combination thereof. In some embodiments, the Mcl-1 inhibitor is AMG-176, MIK665, S63845, or any combination thereof.

In some embodiments, the autophagy inhibitor is chloroquine, 3-methyladenine, hydroxychloroquine, spautin-1, SAR405, bafilomycin A1, 5-amino-4-imidazole carboxamide riboside (AICAR), okadaic acid, autophagy-suppressive algal toxins, analogues of CAMP, LY204002, N6-mercaptopurine riboside, vinblastine, a ULK1 inhibitor, a VPS inhibitor or any combination thereof. In some embodiments, the ULK1 inhibitor is a ULK1/2 inhibitor. In some embodiments, the ULK inhibitor is ULK-101, MRT68921, SBI-0206965, MRT67307, MRT68920, MRT68922, MRT199665, LY3009120, Dorsomorphin, or any combination thereof. In some embodiments, the VPS inhibitor is PIK-III, VPS34-IN1, SAR405, Spautin-1, NSC185058, or any combination thereof. In some embodiments, the macropinocytosis inhibitor is EIPA (ethylisopropylamiloride), Wortmannin, Amiloride, Apilimod, Dyngo-4a, Latrunculin B, or any combination thereof.

In some embodiments, the Wnt/Beta-catenin pathway inhibitor is a beta-catenin inhibitor, a PORCN inhibitor, a GSK3 inhibitor, a CLK inhibitor, or any combination thereof. In some embodiments, the beta-catenin inhibitor is tegavivant, foscenvivant, PRI-724 (also known as ICG-001), C-82, BC2059, or any combination thereof. In some embodiments, the PORCN inhibitor is LGK974 (WNT974), ETC-1922159, CGX1321, CWP232291, or any combination thereof. In some embodiments, the GSK3 inhibitor is Tideglusib, laduviglusib, LiCl (Lithium chloride), CHIR99021, SB216763, AZD1080, LY2090314, or any combination thereof. In some embodiments, the CLK inhibitor is SM08502, SM04690, TG003, KH-CB19, T3.5, CX-4945, or any combination thereof. In some embodiments, the JAK/STAT pathway inhibitor is an inhibitor of JAK1, JAK2, JAK3, STAT3, STAT5, or any combination thereof. In some embodiments, the JAK inhibitor is Ruxolitinib, Fedratinib, Tofacitinib, Baricitinib, or any combination thereof. In some embodiments, the STAT inhibitor is SD-36, Stattic, S31-201, OPB-31121, Napabucasin (BBI608), or any combination thereof.

In some embodiments, the epigenetic modulator is a HDAC inhibitor, a BET inhibitor, an EZH2 inhibitor, a Co-REST inhibitor, an EP300 inhibitor, an LSD1 inhibitor, a PRMT5 inhibitor, an MAT2A inhibitor, a DOTL1 inhibitor, or any combination thereof. In some embodiments, the farnesyl transferase inhibitor is tipifarnib, lonafarnib, rilapladib, or any combination thereof. In some embodiments, the TGFbeta inhibitor is galunisertib (LY2157299), vactosertib (TEW-7197), Fresolimumab, Lerdelimumab, Trabedersen, curcumin, resveratrol, or any combination thereof. In some embodiments, the HSP90 inhibitor is geldanamycin or a derivative (e.g., 17-AAG, 17-DMAG), KOS 953, Radicicol or a derivative (e.g., PU-H71), SNX-2112, Ganetespib, AT13387, Onalespib, Luminespib, KW-2478, or any combination thereof. In some embodiments, the GPX4 inhibitor is RSL3, ML162, DPI7, FINO2, MCB-613, CBS9106, ML210, ODSH, TLN232, or any combination thereof. In some embodiments, the NRF2 inhibitor is ML385, Brusatol, CDDO-Im, RTA-408, trigonelline, or any combination thereof. In some embodiments, the TEAD inhibitor is VT-107, a pan-TEAD inhibitor, VT-104, Verteporfin, CA3, a Statin, K-975, IAG933 or any combination thereof. In some embodiments, the notch/gamma secretase inhibitor is nirogacestat. In some embodiments, the hedgehog inhibitor is Vismodegib, Sonidegib, Glasdegib, or any combination thereof.

In some embodiments, the chemotherapeutic is FOLFOX, FOLFIRI, 5-FU, Tipircil, trifluridine, TMZ, docetaxel, gemcitabine, abraxane, paclitaxel, cisplatin, carboplatin, etoposide, or any combination thereof.

In some embodiments, the immunotherapy is an immune checkpoint inhibitor, a cytokine inhibitor, a cytokine, a vaccine, an antibody therapy, a bispecific antibody, a cellular therapy, or any combination thereof. In some embodiments, immunotherapy is described in Table 1. In some embodiments, the one or more additional therapeutic agent comprises an immune checkpoint inhibitor and chemotherapeutic agent.

In some embodiments, one or more additional therapeutic agent comprises a COX1/2 inhibitor or a COX1 inhibitor. In some embodiments, the one or more additional therapeutic agent comprises an xCT inhibitor. In some embodiments, the inhibitor xCT inhibitor is a SLCA11 inhibitor.

In some embodiments, the one or more additional therapeutic agent comprises a PPAR agonist.

In some embodiments, the one or more additional therapeutic agent comprises a menin inhibitor.

In some embodiments, the one or more additional therapeutic agent comprises a PTEN stabilizer.

In some embodiments, the one or more additional therapeutic agent comprises an SGK inhibitor.

In some embodiments, the one or more additional therapeutic agent comprises a myc inhibitor.

In some embodiments, the one or more additional therapeutic agent comprises a DLL3 inhibitor.

In some embodiments, the one or more additional therapeutic agent comprises a CCR8 inhibitor.

In some embodiments, the one or more additional therapeutic agent comprises a Sting agonist.

In some embodiments, the one or more additional therapeutic agent comprises a SCD1 inhibitor.

In some embodiments, the one or more additional therapeutic agent comprises a GSPT1 inhibitor. In some embodiments, the inhibitor is MRT-2359.

In some embodiments, the one or more additional therapeutic agent comprises a NEK7 modulator.

In some embodiments, the one or more additional therapeutic agent comprises a hormone receptor modulator.

In some embodiments, the one or more additional therapeutic agents comprises a CTLA-4 inhibitor. In some embodiments, the CTLA-4 inhibitor is botensilimab. In some embodiments, the combination further comprises an immune check point inhibitor. In some embodiments, the immune checkpoint inhibitor is balstilimab.

In some embodiments, the combination comprises an EGFR inhibitor and pembrolizumab.

In some embodiments, the combination comprises a SHP2 inhibitor, an immune checkpoint inhibitor and CTLA-4 inhibitor.

In some embodiments, the combination further comprises one or more pharmaceutically acceptable excipients.

In some embodiments, the RAS(ON) inhibitor(s) and the one or more additional therapeutic agent(s) are administered simultaneously.

In some embodiments, the RAS(ON) inhibitor(s) and the one or more additional therapeutic agent(s) are administered sequentially.

In some embodiments, the RAS(ON) inhibitor(s) and the one or more additional therapeutic agent(s) are formulated in a single composition.

In some embodiments, the RAS(ON) inhibitor(s) and the one or more additional therapeutic agent(s) are formulated in separate compositions.

It is specifically contemplated that any limitation discussed with respect to one embodiment of the disclosure may apply to any other embodiment of the disclosure. Furthermore, any compound or composition of the disclosure may be used in any method of the disclosure, and any method of the disclosure may be used to produce or to utilize any compound or composition of the disclosure.

### BRIEF DESCRIPTION OF THE FIGURES

FIG. 1 graphically depicts the RMC-6291 plus RMC-6236 combination doublet overcomes resistance and prolongs durability in KRAS^{G12C} NSCLC xenograft models. In the graph titled “improved responses,” each condition tested shows, from left to right, RMC-6291 100 or 200^{#} mg/kg po qd, RMC-6236 25 mg/kg po qd, and the combination.

FIG. 2 shows the RMC-6291 plus RMC-6236 combination therapy induces apoptosis, drives durable tumor regressions & forestalls resistance in a KRAS^{G12C} NSCLC xenograft model.

FIG. 3 shows RMC-6291 plus RMC-6236 provides a combination benefit in KRAS^{G12C} PDX models.

FIG. 4 graphically depicts the combination benefit of RMC-6236 plus chemotherapy in PDAC KRAS^{AMP} models.

FIG. 5 shows RAS inhibition by RMC-7977 completely suppressed PI3K activity KRAS^{WT} and KRAS^{G12D}, had a moderate inhibitory effect in KRAS^{G12C} and KRAS^{G12V}, and had no effect in KRAS^{G12R} line (left) and RMC-7977 suppresses PIP3 levels only in KRAS^{G12D}-mutant PDAC lines but not in KRAS^{G12R}-mutant PDAC lines (right).

FIG. 6 shows RMC-7977 decreased the levels of pERK (MAPK signaling) in both KRAS^{G12D} and KRAS^{G12R} mutant cell lines (left) and MEK inhibitor decreased the levels of pERK (MAPK signaling) in both KRAS^{G12D} and KRAS^{G12R} mutant cell lines (right).

FIG. 7 shows combination of RMC-9805 with Abemaciclib drives durable complete response in immunocompetent model of PDAC.

### DETAILED DESCRIPTION

The present disclosure relates generally to compositions and methods comprising combination therapies for treating a RAS related disease or disorder. In particular embodiments, the present disclosure provides combination therapies for treating cancers harboring a RAS mutation. In each embodiment, the compositions and methods comprise a RAS(ON) inhibitor therapy (e.g., RMC-6236, RMC-6291 or RMC-9805). In some embodiments, the present disclosure provides compositions and methods comprising a RAS(ON) inhibitor therapy and one or more additional therapeutic agents. The combination therapies of the present disclosure, in certain aspects, synergistically increases the potency of the active agents disclosed herein. The combination therapy of the present disclosure, in certain aspects, provides improved clinical benefit to patients compared to treatment with an active agent disclosed herein as a monotherapy (e.g., an increase in progression-free survival).

### General Methods

The practice of the present disclosure will employ, unless otherwise indicated, conventional techniques of cell culturing, molecular biology (including recombinant techniques), microbiology, cell biology, biochemistry, and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

In this application, unless otherwise clear from context, (i) the terms “a” and “an” mean “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” means including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Those skilled in the art will appreciate that certain inhibitor compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Inhibitor compounds described herein can be asymmetric (e.g., having one or more stereocenters). All stereoisomers, such as enantiomers and diastereomers, are intended unless otherwise indicated. Compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more inhibitor compounds described herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, inhibitor compounds described herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶CI, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, inhibitor compounds may be utilized in any such form, including in any solid form. In some embodiments, compounds may be provided or utilized in hydrate or solvate form.

Those of ordinary skill in the art, reading the present disclosure, will appreciate inhibitor compounds may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

As used herein, the term “administration” refers to the administration of a composition (e.g., a compound, or a preparation that includes a compound as described herein) to a subject or system. Administration also includes administering a prodrug derivative or analog of the compound or pharmaceutically acceptable salt of the compound or composition to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H) (R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

An “amino acid substitution,” as used herein, refers to the substitution of a wild-type amino acid of a protein with a non-wild-type amino acid. Amino acid substitutions can result from genetic mutations and may alter one or more properties of the protein (e.g., may confer altered binding affinity or specificity, altered enzymatic activity, altered structure, or altered function).

The term “combination therapy” refers to a method of treatment including administering to a subject at least two therapeutic agents, optionally as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

As used herein, an “effective amount,” “therapeutically effective” combination, or a “therapeutically effective” amount refers to an amount sufficient to elicit the desired biological response. In the present disclosure the desired biological response is to treat the disease or disorder (e.g., cancer) of the patient (i.e., a subject in need of treatment with a combination therapy), such as slowing progression of the disease or disorder, or reducing presence of the disease or disorder in the patient. It is understood that reference to “a patient” in the present disclosure refers to a patient in need of treatment with a combination of therapeutic agents (e.g., a RAS(ON) inhibitor and an additional therapeutic agent). The precise amount of the first therapeutic agent and the second therapeutic agent administered to a patient will depend on the mode of administration, the type and severity of the disease or disorder and on the characteristics of the subject, such as general health, age, sex, body weight and tolerance to drugs. In some embodiments, the effective amount of the first therapeutic agent and second therapeutic agent of the combination is different from the effective amount of the first therapeutic agent and/or the second therapeutic agent administered as a monotherapy of either. In some embodiments, the effective amount of the first therapeutic agent and the second therapeutic agent of the combination is the same as the effective amount of the first therapeutic agent and the second therapeutic agent administered as a monotherapy of each. In some embodiments, the therapeutically effective combination of the first therapeutic agent and second therapeutic agent refers to an amount of the first therapeutic agent and the second therapeutic agent that is different from the therapeutically effective amount of the first therapeutic agent and the second therapeutic agent administered as a monotherapy. In some embodiments, the therapeutically effective combination of the first therapeutic agent and the second therapeutic agent refers to an amount of the first therapeutic agent and the second therapeutic agent that is the same as the therapeutically effective amount of one of the first therapeutic agent and the second therapeutic agent administered as a monotherapy.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

As used herein, the term “dosage form” refers to a physically discrete unit of a compound (e.g., an inhibitor compound as disclosed herein) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen). The dosages of one or more of the additional therapies (e.g., non-drug treatments or therapeutic agents) may be reduced from standard monotherapy dosages when administered in combination with a RAS(ON) inhibitor. In certain embodiments, doses may be determined empirically from drug combinations and permutations or may be deduced by isobolographic analysis (e.g., Black et al., Neurology 65: S3-S6 (2005)).

The term “disorder” is used in this disclosure to mean, and is used interchangeably with, the terms disease, condition, or illness, unless otherwise indicated.

The term “isomer,” as used herein, means any tautomer, stereoisomer, atropiosmer, enantiomer, or diastereomer of any compound of the disclosure. It is recognized that the compounds of the disclosure can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the disclosure, the chemical structures depicted herein encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, degraders, chemicals, analogs that mimic the binding site of an enzyme, receptor, or other protein. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the MW is less than 800 Da. In some embodiments, the MW is less than 700 Da. In some embodiments, the MW is less than 600 Da. In some embodiments, the range of the MW of the small molecule is between 600 Da and 700 Da, inclusive. In some embodiments, the range of the MW of the small molecule is between 600 Da and 800 Da, inclusive. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid sidechain of a target protein.

In some embodiments, the inhibitor is a degrader, e.g., a molecular entity designed to selectively induce the degradation of target proteins within cells, such as Proteolysis Targeting Chimeras (PROTACs). A protein degrader, such as a PROTAC, typically consists of a bifunctional molecule that simultaneously binds to both the target protein of interest and a component of the cellular degradation machinery, such as an E3 ubiquitin ligase. This dual binding promotes the ubiquitination of the target protein, leading to its recognition by the proteasome and subsequent degradation. The design and optimization of protein degraders may involve structural modifications, linker optimization, and functional characterization to enhance specificity, potency, and selectivity towards the target protein.

The term “mutation” as used herein indicates any modification of a nucleic acid or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications or chromosomal breaks or translocations. In particular embodiments, the mutation results in an amino acid substitution in the encoded protein.

A “patient” or “subject” is a mammal, e.g., a human, mouse, rat, guinea pig, dog, cat, horse, cow, pig, or non-human primate, such as a monkey, chimpanzee, baboon or rhesus.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

As used herein, the term “pharmaceutical composition” refers to a compound, such as a compound of the present disclosure, or a pharmaceutically acceptable salt thereof, formulated together with a pharmaceutically acceptable excipient.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The terms “RAS inhibitor” and “inhibitor of [a]RAS” are used interchangeably to refer to any inhibitor that targets, that is, selectively binds to or inhibits a RAS protein.

As used herein, the term “RAS (OFF) inhibitor” refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS (OFF) inhibitors may also bind to or inhibit the GTP-bound, active state of RAS (e.g., with a lower affinity or inhibition constant than for the GDP-bound, inactive state of RAS). A RAS (OFF) inhibitor may be mutant-selective, such as selective for a G12C, G12D or G12V mutant. A RAS (OFF) inhibitor may be selective for more than one mutant, or selective for one or more mutants and for wild-type (in either situation, a “pan-KRAS (OFF)” inhibitor). Methods of measuring RAS (OFF) inhibition are known in the art.

As used herein, the terms “RAS(ON) multi-selective inhibitor”, “RAS^{MULTI} inhibitor”, “RAS^{MULTI} (ON) inhibitor” or “RAS (MULTI) inhibitor” refer to a RAS inhibitor of at least 3 RAS isoforms, including variants with missense mutations at one of the following positions: 12, 13, 59, 61, or 146. In some embodiments, a RAS(ON) multi-selective inhibitor refers to a RAS inhibitor of at least 3 RAS variants with missense mutations at one of the following positions: 12, 13, and 61. In non-limiting examples, RMC-6236 and RMC-7977 are RAS(ON) multi-selective inhibitors.

As used herein, the term “RAS(ON) mutant-selective inhibitor” refers to a RAS inhibitor of one variant carrying a missense mutation. In a non-limiting example, RMC-6291 is a RAS(ON) mutant-selective inhibitor of RAS^{G12C} (also termed a “RAS(ON) G12C-selective inhibitor”). In another non-limiting example, RMC-9805 is a RAS(ON) mutant-selective inhibitor of RAS^{G12D} (also termed a “RAS(ON) G12D-selective inhibitor”).

The terms “RAS pathway” and “RAS/MAPK pathway” are used interchangeably herein to refer to a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “treatment” (also “treat” or “treating”), in its broadest sense, refers to any administration of a substance (e.g., a compound of the present disclosure) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition. In any treatment method herein, a patient or subject may be in need of such treatment.

The term “tri-complex” refers to having a mechanism of action entailing formation of a high affinity three-component complex between a synthetic ligand (e.g., a RAS(ON) inhibitor) and two intracellular proteins which do not interact under normal physiological conditions: the target protein of interest, RAS, and a widely expressed cytosolic chaperone protein in the cell, cyclophilin A. Such tri-complexes are known in the art. See, e.g., WO 2020/132597, WO 2021/091956, WO 2021/091967, WO 2021/091982, WO 2022/060836, WO 2022/235864, WO 2022/235870, WO 2023/060253, WO 2023/133543, and WO 2023/240263.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

### I. Combination Therapy

Provided herein are compositions comprising a RAS(ON) inhibitor and one or more therapeutic agents for use in treating a RAS related disease or disorder. In certain embodiments, the compositions of the disclosure comprise two or more RAS(ON) inhibitor therapies. In certain embodiments, compositions of the disclosure comprise a RAS(ON) inhibitor therapy and one additional therapeutic agent. In certain embodiments, compositions of the disclosure comprise a RAS(ON) inhibitor therapy and two additional therapeutic agents. In certain embodiments, compositions of the disclosure comprise a RAS(ON) inhibitor therapy and three additional therapeutic agents. In certain embodiments, compositions of the disclosure comprise a RAS(ON) inhibitor therapy and four or more additional therapeutic agents.

Also provided are pharmaceutical compositions including the combinations, or a pharmaceutically acceptable salt thereof, and a pharmaceutically acceptable excipient. Composition comprising a combination of therapeutic agents may be used in methods of modulating RAS (e.g., in a subject or in a cell) and in methods of treating RAS related diseases and disorders, as described herein. The present disclosure provides, inter alia, compositions, methods, and kits for treating or preventing a RAS related disease or disorder (e.g., cancer).

A RAS(ON) inhibitor as disclosed herein may be administered before, after, or concurrently with one or more of such additional therapies. When combined, dosages of a RAS(ON) inhibitor and dosages of the one or more additional therapies (e.g., non-drug treatment or therapeutic agent) provide a therapeutic effect (e.g., synergistic or additive therapeutic effect). A RAS(ON) inhibitor as disclosed herein and an additional therapy, such as an anti-cancer agent, may be administered together, such as in a unitary pharmaceutical composition, or separately and, when administered separately, this may occur simultaneously or sequentially. Such sequential administration may be close or remote in time.

All references herein are incorporated by reference for the agents described, including compound or molecular structures disclosed therein, whether explicitly stated as such or not.

### a) RAS(ON) Inhibitors

Compositions and methods of the present disclosure include a RAS(ON) inhibitor. In certain embodiments, a RAS(ON) inhibitor useful in the present disclosure may form a high affinity three-component complex, or conjugate, between a synthetic ligand and two intracellular proteins which do not interact under normal physiological conditions: the target protein of interest (e.g., RAS), and a widely expressed cytosolic chaperone (presenter protein) in the cell (e.g., cyclophilin A). More specifically, in some embodiments, the inhibitors of RAS described herein induce a new binding pocket in RAS by driving formation of a high affinity tri-complex, or conjugate, between the RAS protein and the widely expressed cytosolic chaperone, cyclophilin A (CYPA). In some embodiments, the RAS(ON) inhibitor is a RAS(ON) multi-selective inhibitor (e.g., RMC-6236, RMC-7977 or GFH547). In some embodiments, the RAS(ON) inhibitor is a mutant-selective inhibitor (e.g., RMC-6291 or RMC-9805).

In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). In some embodiments, a RAS(ON) inhibitor has a molecular weight of between 800 and 1200 Da, inclusive. Reference to the term RAS(ON) inhibitor includes, without limitation, any one or more RAS(ON) inhibitors selected from the RAS(ON) inhibitors disclosed in WO 2021/091956, WO 2021/091982, WO 2021/091967, WO 2022/060836, WO 2022/235864, WO 2022/235870, WO 2022/251292, WO 2023/133543, WO 2023/015559, WO 2023/025832, WO 2023/060253, WO 2023/133543, WO 2023/240263 or PCT application serial number PCT/US2023/037057, PCT/US2024/023272, PCT/US2024/023208, or WO 2024/067857, WO 2024/060966, WO 2024/017859, WO 2024/008834, WO 2024/008610, WO 2023/232776, WO 2023/208005, WO 2023/086341, WO 2023/025832, WO 2023/015559, CN 117720556, CN 117720555, CN 117720554, CN 1177534687, CN 11753685, or CN 11753684, each of which is incorporated by reference in its entirety, or a combination of any such RAS(ON) inhibitors. Methods of determining RAS(ON) inhibition are known in the art. See, e.g., WO 2021/091956 and WO 2022/060836.

In some embodiments, the RAS(ON) inhibitor is compound A647 of WO 2021/091982. In some embodiments, the RAS(ON) inhibitor is compound A122 of WO 2022/060836. In some embodiments, the RAS inhibitor therapy comprises two or more RAS(ON) inhibitors.

In some embodiments, the RAS(ON) inhibitor is RMC-6236

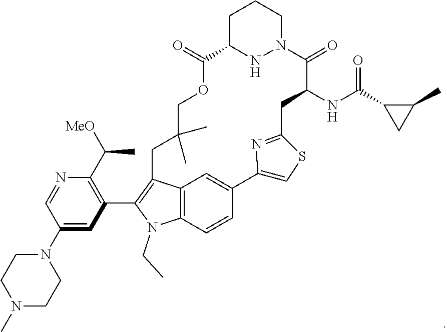

In some embodiments, the RAS(ON) inhibitor is RMC-7977

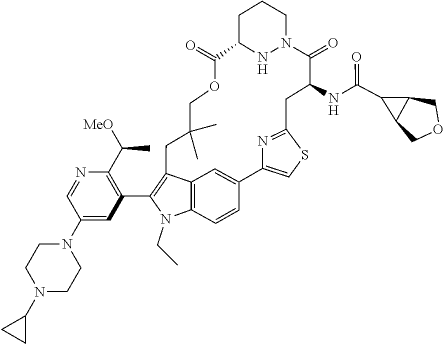

In some embodiments, the RAS(ON) inhibitor is RMC-6291

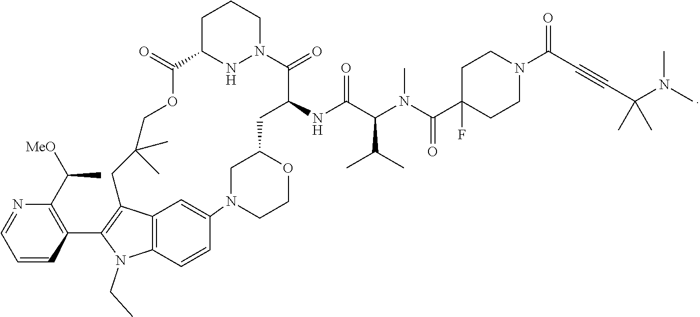

In some embodiments, the RAS(ON) inhibitor is RMC-4998

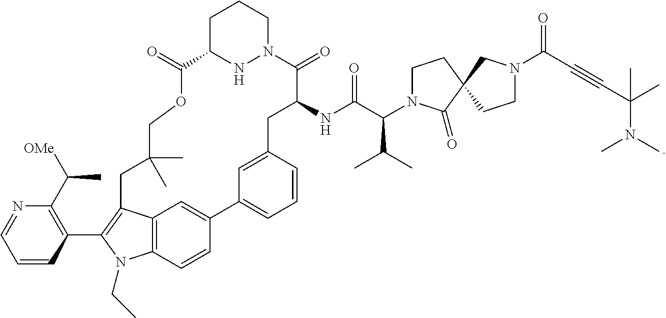

In some embodiments, the RAS(ON) inhibitor is RMC-9805

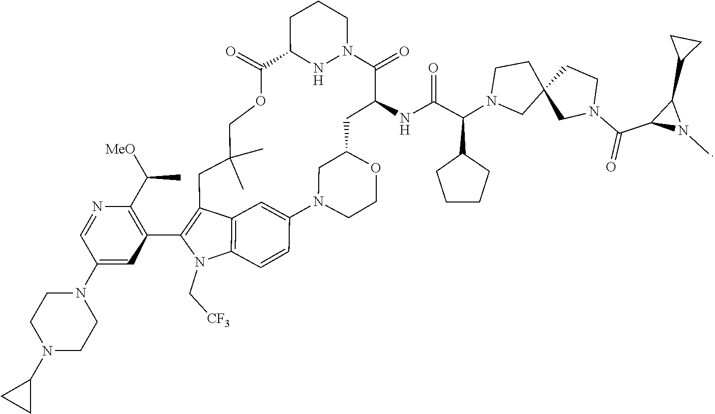

In non-limiting examples, the RAS(ON) inhibitor therapy comprises RMC-6236 and RMC-6291 or RMC-6236 and RMC-9805.

Syntheses of RAS(ON) inhibitors are known, for example, as described in WO 2021/091956, WO 2021/091982 or WO 2022/060836, together with synthetic methods known in the art of synthetic organic chemistry, or variations thereon as appreciated by those skilled in the art.

### b) RAS/MAPK Inhibitors

Compositions and methods described herein may include a RAS(ON) inhibitor (e.g., RMC-6236, RMC-6291 and/or RMC-9805) in combination with one or more RAS/MAPK pathway inhibitors. The RAS/MAPK pathway is a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions. In some embodiments, a therapeutic agent that may be combined with a RAS(ON) inhibitor is an inhibitor of the MAP kinase (MAPK) pathway (or “MAPK pathway inhibitor”). MAPK pathway inhibitors include, but are not limited to, one or more MAPK pathway inhibitors described in Cancers (Basel) 2015 September; 7 (3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLOS One. 2014 Nov. 25; 9 (11)); and GSK1120212 (or JTP-74057, described in Clin Cancer Res. 2011 Mar. 1; 17 (5): 989-1000). The MAPK pathway inhibitor may be PLX8394, LXH254, GDC-5573, or LY3009120. A MAPK pathway inhibitor may be a PI3Ka: RAS breaker, such as BBO-10203.

### i) RAS (OFF) Inhibitors and RAS (OFF) Degraders

Compositions and methods described herein may include a RAS(ON) inhibitor (e.g., RMC-6236, RMC-6291 and/or RMC-9805) in combination with one or more RAS (OFF) inhibitors. Numerous mutant-selective and pan-KRAS inhibitors have been disclosed. A RAS (OFF) inhibitor may be administered or formulated in combination with a RAS(ON) inhibitor described herein. RAS (OFF) inhibitors are designed to inhibit RAS activity by targeting different regions of the RAS protein in its inactive state, preventing its activation and downstream signaling.

In some embodiments, a RAS (OFF) inhibitor is a KRAS (OFF) inhibitor that has a molecular weight of under 700 Da. The term “KRAS (OFF) inhibitor” refers to any RAS (OFF) inhibitor that binds to KRAS in its GDP-bound “OFF” position. In some embodiments, the KRAS (OFF) inhibitor is specific for a KRAS^{G12C} mutation. KRAS^{G12C} (OFF) inhibitors use a covalent binding group that allows them to selectively target the KRAS^{G12C} mutant protein, and many such inhibitors comprise a pyrimidine core. KRAS^{G12C} (OFF) inhibitors all target the same cysteine residue in the KRAS^{G12C} mutant protein, leading to a conformational change that locks the protein in an inactive state. KRAS^{G12C} (OFF) inhibitors include, but are not limited to, AMG510 (sotorasib), MRTX849 (adagrasib), MRTX1257, GDC-6036 (divarasib), JDQ443 (opnurasib), ERAS-3490, LY3537982 (olomorasib), BI 1823911, BPI-421286, JAB-3312, JAB-21000, JAB-21822 (glecirasib), D-1553, D3S-001, HBI-2438, HS-10370, MK-1084, YL-15293, BBO-8520 (ON/OFF inhibitor), FMC-376 (ON/OFF inhibitor), GEC255, BBO-11818, and GFH925 (IBI351). In some embodiments, the KRAS (OFF) inhibitor is selected from AMG510 and MRTX849. In some embodiments, the KRAS (OFF) inhibitor is AMG510. In some embodiments, the KRAS (OFF) inhibitor is selected from BPI-421286, JNJ-74699157 (ARS-3248), LY3537982, MRTX1257, ARS853, ARS1620, or GDC-6036.

In some embodiments, a KRAS (OFF) inhibitor is specific for a KRAS^{G12D} mutation. Many KRAS^{G12D} (OFF) inhibitors have been developed using RAS^{G12C} (OFF) inhibitors as a starting point, thus sharing the backbone of G12C inhibitors in combination with other chemical moieties such as piperazine-based compounds. Non-limiting examples of KRAS^{G12D} (OFF) inhibitors include MRTX1133, MRTX282, JAB-22000, ERAS-4, ERAS-5024, HRS-4642, BI-2852, BI-2852, ASP3082, TH-Z827, TH-Z835, QTX-3046, GFH375 (VS-7375), INCB161734 and KD-8.

In some embodiments, the small molecule RAS (OFF) inhibitor is specific for a KRAS^{G12V} mutation. In some embodiments, the small molecule RAS (OFF) inhibitor is specific for a KRAS^{G13D} mutation. In some embodiments, the small molecule RAS (OFF) inhibitor is a pan-KRAS (OFF) inhibitor. In some embodiments, reference to the term RAS (OFF) inhibitor includes any such RAS (OFF) inhibitor disclosed in any one of the following patent applications: WO 2024085661, WO 2024083258, WO 2024083256, WO 2024083246, WO 2024083168, WO 2024078555, WO 2024076674, WO 2024076672, WO 2024076670, WO 2024067714, WO 2024067575, WO 2024064335, WO 2024063578, WO 2024063576, WO 2024061370, WO 2024061333, WO 2024061267, WO 2024056063, WO 2024055112, WO 2024054926, WO 2024054647, WO 2024054625, WO 2024051763, WO 2024051721, WO 2024050742, WO 2024050640, WO 2024046406, WO 2024046370, WO 2024045066, WO 2024044667, WO 2024044649, WO 2024044334, WO 2024041621, WO 2024041606, WO 2024041589, WO 2024041573, WO 2024040131, WO 2024040109, WO 2024040080, WO 2024036270, WO 2024034657, WO 2024034593, WO 2024034591, WO 2024034123, WO 2024032747, WO 2024032704, WO 2024032703, WO 2024032702, WO 2024031088, WO 2024030647, WO 2024030633, WO 2024029613, WO 2024022507, WO 2024022444, WO 2024020159, WO 2024019103, WO 2024017859, WO 2024017392, WO 2024015731, WO 2024015262, WO 2024012456, WO 2024009191, WO 2024008179, WO 2024008178, WO 2024008068, WO 2024006445, WO 2024006424, WO 2024002373, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023246914, WO 2023246903, WO 2023246777, WO 2023244713, WO 2023244615, WO 2023244604, WO 2023244600, WO 2023244599, WO 2023230190, WO 2023226630, WO 2023225302, WO 2023225252, WO 2023220421, WO 2023219941, WO 2023217148, WO 2023215802, WO 2023215801, WO 2023213269, WO 2023212548, WO 2023208005, WO 2023205719, WO 2023199180, WO 2023198191, WO 2023197984, WO 2023190748, WO 2023185864, WO 2023183755, WO 2023183585, WO 2023179703, WO 2023179629, WO 2023173017, WO 2023173016, WO 2023173014, WO 2023172737, WO 2023171781, WO 2023159087, WO 2023159086, WO 2023154766, WO 2023152255, WO 2023151674, WO 2023151621, WO 2023150394, WO 2023150284, WO 2023143623, WO 2023143605, WO 2023143352, WO 2023143352, WO 2023143312, WO 2023141570, WO 2023141300, WO 2023138662, WO 2023138601, WO 2023138589, WO 2023138524, WO 2023133183, WO 2023133181, WO 2023130012, WO 2023125989, WO 2023125627, WO 2023122662, WO 2023122154, WO 2023120742, WO 2023119677, WO 2023117681, WO 2023116934, WO 2023116895, WO 2023114733, WO 2023105491, WO 2023104018, WO 2023103906, WO 2023103523, WO 2023101928, WO 2023099624, WO 2023099624, WO 2023099620, WO 2023099612, WO 2023099608, WO 2023099592, WO 2023098832, WO 2023098425, WO 2023097227, WO 2023081840, WO 2023081476, WO 2023078424, WO 2023077441, WO 2023072297, WO 2023072188, WO 2023066371, WO 2023064857, WO 2023061463, WO 2023061294, WO 2023057985, WO 2023056951, WO 2023056421, WO 2023051586, WO 2023049697, WO 2023046135, WO 2023045960, WO 2023041059, WO 2023041059, WO 2023040989, WO 2023040513, WO 2023039240, WO 2023039020, WO 2023036282, WO 2023034290, WO 2023030517, WO 2023030495, WO 2023030385, WO 2023030495, WO 2023030517, WO 2023030685, WO 2023030687, WO2023034290, WO 2023036282, WO 2023039240, WO 203020347, WO 2023025116, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023040989, WO 2023039240, WO 2023039020, WO 2023036282, WO 2023034290, WO 2023030517, WO 2023030495, WO 2023030385, WO 2023025116, WO 2023020523, WO 2023020521, WO 2023020519, WO 2023020518, WO 2023020347, WO 2023018812, WO 2023018810, WO 2023018809, WO 2023018699, WO 2023014979, WO 2023014006, WO 2023004102, WO 2023003417, WO 2023001141, WO 2023001123, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762 (e.g., Compound 44 or Compound 66a), WO 2022212894, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021113595, WO 2021107160, WO 2021106231, WO 2021088458, WO 2021086833, WO 2021085653, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, CN 115721720, CN 115724842, CN 115785124, CN 115785199, CN 114437084, CN 114195788, CN 114437107, CN 114409653, CN 114380827, CN 114195804, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113248521, CN 113321654, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, and CN 109574871, or CN 117683051, CN 117645627, CN 117624194, CN 117624190, CN 117586280, CN 117486901, CN 117466917, CN 117462688, CN 117362315, CN 117327102, CN 117327094, CN 117327074, CN 117285590, CN 117263959, CN 117247382, CN 117186095, CN 117164605, CN 116969977, CN 116925075, CN 116891489, CN 116731045, CN 116731044, CN 116554208, CN 116514846, CN 116478184, CN 116478141, CN 116410145, CN 116375742, CN 116354988, CN 116332948, CN 116332938, CN 116327956, CN 116262759, CN 116217592, CN 116199703, CN 116162099, CN 116143806, CN 116143805, CN 116120315, CN 116102559, CN 115960105, CN 115894520, CN 115872979, CN 115850267, CN 115785199, CN 115785124, CN 115785124, CN 115724842, CN 115716840, CN 115703775, CN 115611923, CN 115611898, CN 115583937, CN 115572278, CN 115557949, CN 115521312, CN 115504976, CN 115490709, CN 115466272, CN 115433183, CN 115433179, CN 115403575, CN 115385938, CN 115385937, CN 115385912, CN 115381786, CN 115368383, CN 115368382, CN 115368381, CN 115353506, CN 115322158, CN 115304623, CN 115304602, CN 115197245, CN 115181106, CN 114989195, CN 114989166, CN 114989147, CN 114920741, CN 114920739, CN 114907387, CN 114874234, CN 114874201, CN 114716436, CN 114716435, CN 114685532, CN 114685460, CN 114591319, CN 114539293, CN 114539286, CN 114539246, CN 114437107, CN 114437084, CN 114409653, CN 114380827, CN 114195804, CN 114195788, CN 114057776, CN 114057744, CN 114057743, CN 113999226, CN 113980032, CN 113980014, CN 113929676, CN 113754653, CN 113683616, CN 113563323, CN 113527299, CN 113527294, CN 113527293, CN 113493440, CN 113429405, CN 113248521, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, and CN 109574871, each of which is incorporated herein by reference in its entirety, including the RAS compound structures disclosed therein which are specifically incorporated herein by reference.

In any embodiment employing a RAS (OFF) inhibitor herein, a RAS (OFF) degrader targeting the OFF state of RAS may alternatively be employed. These degraders are known in the art. RAS degraders may be found, for example, in one or more of the following applications: WO 2024083258, WO 2024083256, WO 2024055112, WO 2024054625, WO 2024050742, WO 2024044334, WO 2024040080, WO 2024034657, WO 2024034593, WO 2024034591, WO 2024034123, WO 2024029613, WO 2024020159, WO 2024019103, WO 2024017392, WO 2023185864, WO 2023171781, WO 2023141570, WO 2023138524, WO 2023130012, WO 2023116934, WO 2023099620, WO 2023081476,

WO 2023077441, and CN 115785199, each of which is incorporated herein by reference in its entirety. In some embodiments, the RAS (OFF) inhibitor is a peptide-based inhibitor. Peptide-based RAS (OFF) inhibitors have been developed that target specific regions of the RAS protein, such as the Switch II region or the RAS-effector interface. Non-limiting examples include the K-Ras-binding peptide (Krpep-2d), the Ras inhibitory peptide (RasIn) and LUNA18 (NCT05012618). Peptide-based RAS (OFF) inhibitors are a class of compounds that target the RAS protein by disrupting its interaction with its downstream effectors or other signaling proteins. These inhibitors are typically designed to mimic the binding motifs of RAS-interacting proteins or other RAS effectors, such as RAF or PI3K. By binding to RAS at the same site as these effectors, peptide-based inhibitors can effectively compete with these proteins and prevent the activation of downstream signaling pathways.

Peptide-based RAS (OFF) inhibitors can be further classified into two main categories: those that target the RAS-effector interface, and those that target other regions of the RAS protein. Peptide-based inhibitors that target the RAS-effector interface are designed to bind to the switch regions of RAS that are critical for its interaction with downstream effectors, such as RAF or PI3K. These inhibitors typically contain amino acid residues that are similar to those found in the binding motifs of RAS-interacting proteins or effectors and are often designed to form hydrogen bonds or other interactions with key residues on the surface of RAS.

Peptide-based RAS (OFF) inhibitors that target other regions of the RAS protein are typically designed to disrupt other interactions that are critical for the activation or signaling of RAS. For example, some peptide-based inhibitors are designed to bind to the hypervariable region of RAS, which is thought to play a role in membrane localization and anchoring of the protein. By binding to this region, peptide-based inhibitors can prevent the proper localization of RAS to the plasma membrane, which is necessary for its activation and signaling.

Several common motifs have been identified as important for the binding of RAS-interacting proteins and effectors and are often used in the design of peptide-based inhibitors. One example is the RAF-binding domain (RBD), which is found in many RAS-interacting proteins and is important for the interaction of RAS with downstream effectors such as RAF. The RBD contains a conserved amino acid sequence (Arg-Xaa-Arg) that is critical for binding to RAS, and this motif has been incorporated into several peptide-based inhibitors designed to disrupt the RAS-RAF interaction. Another example is the RAS-binding domain (RBD) of PI3K, which is important for the interaction of RAS with this downstream effector. The RBD of PI3K contains several conserved amino acid residues (such as Arg-Arg-Trp) that are critical for binding to RAS, and these motifs have been used in the design of peptide-based inhibitors that target the RAS-PI3K interaction. Other common motifs used in peptide-based RAS (OFF) inhibitors include the Ras-binding domain (RBD) of other RAS-interacting proteins such as RaIGDS and SOS, as well as sequences that mimic the structure of the switch regions of RAS itself. These motifs are typically used to optimize the binding affinity and selectivity of the inhibitor for the desired target protein or interaction.

In some embodiments, the RAS (OFF) inhibitor is an antibody or antigenic binding peptide specific for RAS (OFF). Antibodies have been developed that bind to specific regions of the RAS protein, such as the Switch II region or the RAS-effector interface. For example, some antibodies have been developed that target the switch regions of RAS proteins, which are critical for the activation of these proteins and their interaction with downstream effectors. Binding of these antibodies to the switch regions can prevent the conformational changes required for RAS activation and downstream signaling. Another approach involves the use of antibodies that target RAS-interacting proteins or downstream effectors, such as RAF or PI3K. Binding of these antibodies to their target proteins can disrupt the RAS-dependent signaling pathways and inhibit the growth and survival of cancer cells. Additionally, some antibodies have been developed that can induce the internalization and degradation of RAS proteins, leading to their depletion and inhibition of downstream signaling. For example, some antibodies have been developed that recognize the unique structure of mutant RAS proteins and target them for degradation via the ubiquitin-proteasome pathway. Non-limiting examples of KRAS (OFF)-specific inhibitory antibodies include anti-p21ser, and K27 (DARPin) (see, e.g., Khan et al, Biochim Biophys Acta Mol Cell Res. 2020 February; 1867 (2): 118570).

### ii) SOS1 Inhibitors

In some embodiments, compositions and methods described herein may include a RAS(ON) inhibitor (e.g., RMC-6236, RMC-6291 and/or RMC-9805) in combination with one or more SOS1 inhibitors. A SOS1 inhibitor may be administered or formulated in combination with a RAS(ON) inhibitor and/or any additional therapeutic agent described herein. In some embodiments, a SOS1 inhibitor is one or more of RMC-5845, RMC-4948, RMC-0331, BI-1701963, BI-3406, SDR5, MRTX-0902, and BAY-293. In some embodiments, reference to the term SOS1 inhibitor includes any such SOS1 inhibitor disclosed in any one of the following patent applications: WO 2023029833, WO 2023041049, WO 2023022497, WO 2022184116, WO 2022170952, WO 2022170917, WO 2022171184, WO 2022170802, WO 2022161461, WO 2022121813, WO 2022028506, WO 2022139304, WO 2021228028, WO 2019122129, CN 115215847, CN 115028644, CN 114685488, CN 111393519, each of which is incorporated herein by reference in its entirety, including the compound structures disclosed therein which are specifically incorporated herein by reference.

iii) SHP Inhibitors

In some embodiments, compositions and methods described herein may include a RAS(ON) inhibitor (e.g., RMC-6236, RMC-6291 and/or RMC-9805) in combination with one or more SHP inhibitors. A SHP inhibitor may be administered or formulated in combination with a RAS(ON) inhibitor and/or any additional therapeutic agent described herein. In some embodiments, the SHP inhibitor is an inhibitor of SHP1. In some embodiments, the SHP inhibitor is an inhibitor of SHP2. In some embodiments, the SHP 1 inhibitor is SB6299 aka DA-4511. In some embodiments, a SHP2 inhibitor is one or more of SHP099, TNO155, RMC-4550, RMC-4630, JAB-3068, JAB-3312, RLY-1971, ERAS-601, SH3809, PF-07284892, or BBP-398. In some embodiments, reference to the term SHP2 inhibitor includes any such SHP2 inhibitor disclosed in any one of the following patent applications: WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2022063190, WO 2022043865, WO 2022042331, WO 2022033430, WO 2022017444, WO 2022007869, WO 2021259077, WO 2021249449, WO 2021249057, WO 2021244659, WO 2021218755, WO 2021176072, WO 2021171261, WO 2021149817, WO 2021148010, WO 2021147879, WO 2021143823, WO 2021143701, WO 2021143680, WO 2021281752, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Pat. Nos. 11,179,397, 11,044,675, 11,034,705, 11,033,547, 11,001,561, 10,988,466, 10,954,243, 10,934,302, or 10,858,359, each of which is incorporated herein by reference in its entirety, including the compound structures disclosed therein which are specifically incorporated herein by reference.

### iv) MEK Inhibitors

In some embodiments, compositions and methods described herein may include a RAS(ON) inhibitor (e.g., RMC-6236, RMC-6291 and/or RMC-9805) in combination with one or more MEK inhibitors. A MEK inhibitor may be administered or formulated in combination with a RAS(ON) inhibitor and/or any additional therapeutic agent described herein. In some embodiments, a MEK inhibitor is one or more of pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N. In some embodiments, reference to the term MEK inhibitor includes any such MEK inhibitor disclosed in any one of the following patent applications: WO 2022221866, WO 2022125941, WO 2022208391, WO 2022015736, WO 2022177557, WO 2021018866, WO 2021069486, WO 2021142144, WO 2021168283, WO 2021234097, WO 2019076947, WO 2018233696, WO 2016188472, WO 2014063024, WO 2013019906, WO 2011047238, WO 2007044515, US2023032403, and CN 115813930, each of which is incorporated herein by reference in its entirety, including the compound structures disclosed therein which are specifically incorporated herein by reference.

### v) RAF Inhibitors

In some embodiments, compositions and methods described herein may include a RAS(ON) inhibitor (e.g., RMC-6236, RMC-6291 and/or RMC-9805) in combination with one or more RAF inhibitors. A RAF inhibitor may be administered or formulated in combination with a RAS(ON) inhibitor and/or any additional therapeutic agent described herein. In some embodiments, a RAF inhibitor is VS-6766. In some embodiments, a RAF inhibitor is a BRAF inhibitor. BRAF inhibitors that may be used in combination with a RAS(ON) inhibitor include, for example, Vs6766, IK-595, vemurafenib, dabrafenib, and encorafenib. BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E. In some embodiments, reference to the term RAF inhibitor includes any such RAF inhibitor disclosed in any one of the following patent applications: WO 2022226626, WO 2022226261, WO 2019084459, WO 2018203219, WO 2017212442, WO 2015075483, WO 2013134243, WO 2013134298, WO 2011047238, WO 2011025965, WO 2011025947, WO 2011025951, WO 2011025940, WO 2011025938, WO 2010065893, WO 2009016460, WO 2009130015, WO 2009111278, WO 2009111279, WO 2008028141, and WO 2006024834, each of which is incorporated herein by reference in its entirety, including the compound structures disclosed therein which are specifically incorporated herein by reference.

### vi) ERK Inhibitors

In some embodiments, compositions and methods described herein may include a RAS(ON) inhibitor (e.g., RMC-6236, RMC-6291 and/or RMC-9805) in combination with one or more ERK inhibitors. An ERK inhibitor may be administered or formulated in combination with a RAS(ON) inhibitor and/or any additional therapeutic agent described herein. In some embodiments, an ERK inhibitor is an ERK1/2 inhibitor, such as ERAS-007. In some embodiments, an ERK inhibitor is an ERK 5 inhibitor. In some embodiments, an ERK inhibitor is one or more of ASTX-029 or I-75. In some embodiments, reference to the term ERK inhibitor includes any such ERK inhibitor disclosed in any one of the following patent applications: WO 2021110169, WO 2021110168, WO 2021252316, WO 2020102686, WO 2020228817, WO 2020107987, WO 2019233456, WO 2019233457, WO 2016025561, WO 2016192063, WO 2016106029, WO 2016106009, WO 2015051341, WO 2014124230, WO 2014052563, WO 2011041152, WO 200910550, WO 2008153858, CN114315837, CN 115057860, and CN 107973783, each of which is incorporated herein by reference in its entirety, including the compound structures disclosed therein which are specifically incorporated herein by reference.

vii) MAPK Inhibitors

In some embodiments, compositions and methods described herein may include a RAS(ON) inhibitor (e.g., RMC-6236, RMC-6291 and/or RMC-9805) in combination with one or more Mitogen-Activated Protein Kinase (MAPK) inhibitors. A MAPK inhibitor may be administered or formulated in combination with a RAS(ON) inhibitor and/or any additional therapeutic agent described herein. In some embodiments, a MAPK inhibitor is a p38MAPK inhibitor or a MAP3K8 inhibitor. In some embodiments, the MAPK inhibitor is one or more of Tilpisertib (GS-4875) and neflamapidmod (VX-745). In some embodiments, reference to the term MAPK inhibitor includes any such MAPK inhibitor disclosed in any one of the following patent applications: WO 2016029263, CN 114767674, CN 115850179, and CN 1743006, each of which is incorporated herein by reference in its entirety, including the compound structures disclosed therein which are specifically incorporated herein by reference.

In some embodiments, a therapeutic agent that may be combined with a RAS(ON) inhibitor is an inhibitor of MAP2K4. A non-limiting example of a MAP2K4 inhibitor useful according to the disclosure is HRX-0233.

### c) Kinase Inhibitors

Compositions and methods described herein may include a RAS(ON) inhibitor (e.g., RMC-6236, RMC-6291 and/or RMC-9805) in combination with one or more kinase inhibitors. Tyrosine kinases and serine/threonine kinases play a crucial role in various cellular processes such as cell signaling, growth, and differentiation. Kinase inhibitors known in the art have been developed as a treatment for various types of cancer in addition to therapies for conditions such as neurodegenerative diseases, autoimmune disorders, and inflammation.

### i) PKA Inhibitors

In some embodiments, compositions and methods described herein may include one or more Protein Kinase A (PKA) inhibitors. A PKA inhibitor may be administered or formulated in combination with a RAS(ON) inhibitor and/or any additional therapeutic agent described herein. In some embodiments, a PKA inhibitor is H89. In some embodiments, reference to the term PKA inhibitor includes any such PKA inhibitor disclosed in any one of the following patent applications: CN 106620678 and CN 114632155, each of which is incorporated herein by reference in its entirety, including the compound structures disclosed therein which are specifically incorporated herein by reference.

### ii) FAK Inhibitors

In some embodiments, compositions and methods described herein may include a RAS(ON) inhibitor (e.g., RMC-6236, RMC-6291 and/or RMC-9805) in combination with one or more Focal Adhesion Kinase (FAK) inhibitors. A FAK inhibitor may be administered or formulated in combination with a RAS(ON) inhibitor and/or any additional therapeutic agent described herein. In some embodiments, a FAK inhibitor is one or more of BI853520, defactinib, GSK2256098, PF-00562271, and VS-4718. In some embodiments, reference to the term FAK inhibitor includes any such FAK inhibitor disclosed in any one of the following patent applications: WO 2022152315, WO 2021098679, WO 2020135442, WO 2020191448, WO 2012022408, WO 2013134353, WO 2012110774, WO 2010062578, CN 111072571, and KR 101691536, each of which is incorporated herein by reference in its entirety, including the compound structures disclosed therein which are specifically incorporated herein by reference.

iii) ROCK Inhibitor

In some embodiments, compositions and methods described herein may include a RAS(ON) inhibitor (e.g., RMC-6236, RMC-6291 and/or RMC-9805) in combination with one or more Rho-associated, coiled-coil containing protein kinase (ROCK) inhibitors. A ROCK inhibitor may be administered or formulated in combination with a RAS(ON) inhibitor and/or any additional therapeutic agent described herein. In some embodiments, a ROCK inhibitor is GSK269962A. In some embodiments, reference to the term ROCK inhibitor includes any such ROCK inhibitor disclosed in any one of the following patent applications: WO 2023051753, WO 2022237892, WO 2022012409, WO 2021093795, WO 2021214200, WO 2020177292, WO 202011751, WO 2019014304, WO 2019179525, WO 2019089868, WO 2019014300, WO 2018108156, WO 2018009627, WO 2018009625, WO 2018009622, WO 2017123860, WO 2017205709, WO 2016112236, WO 2014068035, WO 2013030367, WO 2012146724, WO 2012067965, WO 2011107608, CN 108129453, CN 108191821, CN 110917352, CN 108558823, CN108047193, CN107973777, CN108047197, CN108129448, CN 115869304, and GB202214708, each of which is incorporated herein by reference in its entirety, including the compound structures disclosed therein which are specifically incorporated herein by reference.

### iv) MSK1 Inhibitors

In some embodiments, compositions and methods described herein may include a RAS(ON) inhibitor (e.g., RMC-6236, RMC-6291 and/or RMC-9805) in combination with one or more Mitogen- and stress-activated kinase (MSK1) inhibitors. A MSK1 inhibitor may be administered or formulated in combination with a RAS(ON) inhibitor and/or any additional therapeutic agent described herein. In some embodiments, a MSK1 inhibitor is one or more of SB-747651A, SB 747651A, Ro 320432, CGP 57380, GSK2830371, SR1664, LY-3214996, PFI-4, MSC-2363318A, and AS601245.

### v) RSK Inhibitors

In some embodiments, compositions and methods described herein may include a RAS(ON) inhibitor (e.g., RMC-6236, RMC-6291 and/or RMC-9805) in combination with one or more ribosomal S6 kinase (RSK) inhibitors. A RSK1 inhibitor may be administered or formulated in combination with a RAS(ON) inhibitor and/or any additional therapeutic agent described herein. In some embodiments, a RSK inhibitor is one or more of BI-D1870, LJH685, SL0101-1, FMK, BRD7389, BIX 02565, LJI308, LJI308-S, LJI308-1, and LJH685-S. In some embodiments, a RSK inhibitor is PMD-026. In some embodiments, reference to the term RSK inhibitor includes any such RSK inhibitor disclosed in any one of the following patent applications: WO 2021249558, WO 2020165646, WO 2017141116, and CN 113801139, each of which is incorporated herein by reference in its entirety, including the compound structures disclosed therein which are specifically incorporated herein by reference.

### vi) ALK Inhibitors

In some embodiments, compositions and methods described herein may include a RAS(ON) inhibitor (e.g., RMC-6236, RMC-6291 and/or RMC-9805) in combination with one or more Anaplastic Lymphoma Kinase (ALK) inhibitors. An ALK inhibitor may be administered or formulated in combination with a RAS(ON) inhibitor and/or any additional therapeutic agent described herein. In some embodiments, an ALK inhibitor is one or more of Crizotinib (Xalkori), Ceritinib (Zykadia), Alectinib (Alecensa), Brigatinib (Alunbrig), Lorlatinib (Lorbrena), Ensartinib (X-396), TAE684, ASP3026, TPX-0131, LDK378 (Ceritinib analog), CEP-37440; 4SC-203, TL-398, PLB1003, TSR-011, CT-707, TPX-0005, and AP26113. Additional examples of ALK kinase inhibitors are described in examples 3-39 of WO05016894.

In some embodiments, reference to the term ALK inhibitor includes any such ALK inhibitor disclosed in any one of the following patent applications: WO 2019142095, WO 2019179482, WO 2018130928, WO 2018127184, WO 2017101803, WO 2016192132, WO 2014100431, WO 2012082972, CN 111138492, CN 110526914, CN 109836415, CN 105801603, CN107987056, and CN 105878248, each of which is incorporated herein by reference in its entirety, including the compound structures disclosed therein which are specifically incorporated herein by reference.

### d) Receptor Tyrosine Kinase Inhibitors

Compositions and methods described herein may include a RAS(ON) inhibitor (e.g., RMC-6236, RMC-6291 and/or RMC-9805) in combination with one or more receptor tyrosine kinase inhibitors. A receptor tyrosine kinase (RTK) inhibitor is a type of molecule (e.g., small molecule, antibody, and nucleic acid) that binds to and blocks the activity of receptor tyrosine kinases or their ligands. RTKs are proteins found on the surface of cells that play a critical role in cell signaling and growth and have been developed as therapeutics for a range of diseases, including cancer, diabetes, and autoimmune disorders. In some embodiments, a therapeutic agent may be a pan-RTK inhibitor, such as afatinib.

### i) EGFR Inhibitors

In some embodiments, compositions and methods described herein may include a RAS(ON) inhibitor (e.g., RMC-6236, RMC-6291 and/or RMC-9805) in combination with one or more EGFR inhibitors. An EGFR inhibitor may be administered or formulated in combination with a RAS(ON) inhibitor and/or any additional therapeutic agent described herein. EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15: 59 (8): 1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), Lazertinib, erlotinib (Tarceva®), and lapatinib (TykerB®). See, e.g., Yan et al., Pharmacogenetics and Pharmacogenomics In Oncology Therapeutic Antibody Development, BioTechniques 2005, 39 (4): 565-8; and Paez et al., EGFR Mutations In Lung Cancer Correlation With Clinical Response To Gefitinib Therapy, Science 2004, 304 (5676): 1497-500. In some embodiments, the EGFR inhibitor is osimertinib (Tagrisso®). In some embodiments, an EGFR inhibitor is one or more of cetuximab, gefitinib (Iressa), erlotinib (Tarceva), and afatinib (Gilotrif). Additional non-limiting examples of small molecule EGFR inhibitors include any of the EGFR inhibitors described in Traxler et al., Exp. Opin. Ther. Patents 1998, 8 (12): 1599-1625. An EGFR inhibitor may be ERAS-801. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4). In some embodiments, the EGFR inhibitor may be bosutinib, crizotinib, dasatinib, erlotinib, gefitinib, lapatinib, pazopanib, ruxolitinib, sunitinib, vemurafenib, abrocitinib, asciminib, futibatinib, ibrutinib, imatinib, pacritinib, or sorafenib. In some embodiments, reference to the term EGFR inhibitor includes any such EGFR inhibitor disclosed in any one of the following patent applications: WO 2023041071, WO 2023049312, WO 2023020600, WO 2023284747, WO 2022206797, WO 2022258977, WO 2022033416, WO 2022033410, WO 2022105908, WO 2022100641, WO 2022014639, WO 2022007841, WO 2021018009, WO 2021057882, WO 2021252661, WO 2021018003, WO 2021073498, WO 2021238827, WO 2020254547, WO 2020216371, WO 2020147838, WO 2020207483, WO 2020254572, WO 2020001350, WO 2021001351, WO 2019164948, WO 2019218958, WO 2019046775, WO 2019015655, WO 2018121758, WO 2018218963, WO 2017220007, WO 2017205459, WO 2017161937, WO 2016192609, WO 199633980, WO 199630347, WO 199730034, WO 199730044, WO 199738994, WO 199749688, WO 199802434, WO 199738983, WO 199519774, WO 199519970, WO 199713771, WO 199802437, WO 199802438, WO 199732881, WO 199833798, WO 199732880, WO 199732880, WO 199702266, WO 199727199, WO 199807726, WO 1997/34895, WO 199631510, WO 199814449, WO 199814450, WO 199814451, WO 199509847, WO 199719065, WO 199817662, WO 199935146, WO 199935132, WO 199907701, WO 199220642, DE 19629652, EP 682027, EP 837063, EP 0787772, EP 0520722, EP 0566226, CN 115960018, CN 110283162, CN 114044774, CN111973601, CN 111973602, and CN113896744, each of which is incorporated herein by reference in its entirety, including the compound structures disclosed therein which are specifically incorporated herein by reference.

ii) HER2 inhibitors

In some embodiments, compositions and methods described herein may include a RAS(ON) inhibitor (e.g., RMC-6236, RMC-6291 and/or RMC-9805) in combination with one or more HER2 inhibitors. A HER2 inhibitor may be administered or formulated in combination with a RAS(ON) inhibitor and/or any additional therapeutic agent described herein. In some embodiments, an HER2 inhibitor is one or more of tucatinib, rastuzumab (Herceptin), pertuzumab (Perjeta), lapatinib (Tykerb), ado-trastuzumab emtansine (Kadcyla), and neratinib (Nerlynx). Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327. In some embodiments, reference to the term HER2 inhibitor includes any such HER2 inhibitor disclosed in any one of the following patent applications: WO 2021156178, WO 2021156180, WO 2021213800, WO 2021088987, WO 2013561183, and WO 2013056108, each of which is incorporated herein by reference in its entirety, including the compound structures disclosed therein which are specifically incorporated herein by reference.

iii) MET Inhibitors

In some embodiments, compositions and methods described herein may include a RAS(ON) inhibitor (e.g., RMC-6236, RMC-6291 and/or RMC-9805) in combination with one or more MET inhibitors. A MET inhibitor may be administered or formulated in combination with a RAS(ON) inhibitor and/or any additional therapeutic agent described herein. In some embodiments, a MET inhibitor is one or more of Crizotinib (Xalkori), Cabozantinib (Cometriq, Cabometyx), Capmatinib (Tabrecta), Tepotinib (Tepmetko), Savolitinib (Volitinib), Onartuzumab (MetMab), Foretinib (GSK1363089), MGCD-265 (Amuvatinib), SU11274, and SU5416. In some embodiments, reference to the term MET inhibitor includes any such MET inhibitor disclosed in any one of the following patent applications: WO 2022226168, WO 2021222045, WO 2020047184, WO 2020015744, WO 2020244654, WO 2020156453, WO 2019206268, WO 2018077227, WO 2017012539, WO 2016015653, WO 2016012963, WO 2012015677, WO 2011162835, WO 2010089507, WO 2009091374, WO 2009056692, WO 2008051547, WO 2007130468, US2012237524, CN 103497177, CN 107311983, CN 107382968, CN 110218191, and TW201331206, each of which is incorporated herein by reference in its entirety, including the compound structures disclosed therein which are specifically incorporated herein by reference.

### iv) AXL Inhibitors

In some embodiments, compositions and methods described herein may include a RAS(ON) inhibitor (e.g., RMC-6236, RMC-6291 and/or RMC-9805) in combination with one or more AXL inhibitors. An AXL inhibitor may be administered or formulated in combination with a RAS(ON) inhibitor and/or any additional therapeutic agent described herein. AXL is a receptor tyrosine kinase that belongs to the TAM family of receptors, which also includes TYRO3 and MERTK. In some embodiments, an AXL inhibitor is one or more of bemcentib, BGB324, R428, SGI-7079, TP-0903, BMS-777607, UNC2025, and TP-0903. In some embodiments, reference to the term AXL inhibitor includes any such AXL inhibitor disclosed in any one of the following patent applications: WO 2023045816, WO 2022237843, WO 2022246179, WO 2021012717, WO 2021088787, WO 2021067772, WO 2021239133, WO 2021204713, WO 2020238802, WO 2019039525, WO 2019101178, WO 2019074116, WO 2017146236, WO 2016097918, WO 2015012298, WO 2010005876, WO 2010083465, CN 115073367, and JP 2022171109, each of which is incorporated herein by reference in its entirety, including the compound structures disclosed therein which are specifically incorporated herein by reference.

### v) IGFR Inhibitors

In some embodiments, compositions and methods described herein may include a RAS(ON) inhibitor (e.g., RMC-6236, RMC-6291 and/or RMC-9805) in combination with one or more insulin-like growth factor receptor 1 (IGF-1R) inhibitors. An IGFR inhibitor may be administered or formulated in combination with a RAS(ON) inhibitor and/or any additional therapeutic agent described herein. IGFR inhibitors have been developed to target the IGFR receptor, which plays a critical role in cancer progression and metastasis. In some embodiments, an IGFR inhibitor is one or more of linsitinib, AXL1717, OSI-906 (Linsitinib), BMS-754807, BI 836845, AZ12253801, PQIP (Pyrrolo[1,2-a]quinoxaline), and NVP-AEW541. In some embodiments, reference to the term IGFR inhibitor includes any such IGFR inhibitor disclosed in any one of the following patent applications: WO 2022115946, WO 2022217923, WO 2021203861, WO 2021246413, WO 2020116398, WO 2019046600, WO 2018195250, WO 2018221521, WO 2018204872, WO 2017072196, WO 2016173682, WO 2015162291, WO 2015162292, WO 2010066868, WO 2006069202, and CN 112125916, each of which is incorporated herein by reference in its entirety, including the compound structures disclosed therein which are specifically incorporated herein by reference.

### vi) RET Inhibitors

In some embodiments, compositions and methods described herein may include a RAS(ON) inhibitor (e.g., RMC-6236, RMC-6291 and/or RMC-9805) in combination with one or more Rearranged during transfection (RET) inhibitors. An RET inhibitor may be administered or formulated in combination with a RAS(ON) inhibitor and/or any additional therapeutic agent described herein. RET plays a critical role in various cellular processes, including cell growth, differentiation, survival, and migration. RET is activated by binding of its ligands, such as glial cell line-derived neurotrophic factor (GDNF) family ligands, which leads to the activation of downstream signaling pathways that promote these cellular processes. In some embodiments, a RET inhibitor is one or more of pralsetinib, selpercatinib (LOXO-292), BLU-667, RXDX-105, TPX-0046, GSK3179106, molidustat (BAY 85-3934), and RPI-1 (Retrophin). In some embodiments, reference to the term RET inhibitor includes any such RET inhibitor disclosed in any one of the following patent applications: WO 2021211380, WO 2021057963, WO 2021043209, WO 2021222017, WO 2020035065, WO 2020114487, WO 2020200314, WO 2020200316, WO 2020114494, WO 2018071447, WO 2018213329, WO 2017079140, WO 2014050781, CN 113943285, CN 113683610, CN 113683611, CN 113620944, CN 113620945, CN 113527291, CN 113527292, CN 113527290, CN 113135896, CN 111057075, CN111233899, and CN111362923, each of which is incorporated herein by reference in its entirety, including the compound structures disclosed therein which are specifically incorporated herein by reference.

vii) ROS1 Inhibitors

In some embodiments, compositions and methods described herein may include a RAS(ON) inhibitor (e.g., RMC-6236, RMC-6291 and/or RMC-9805) in combination with one or more c-ros oncogene 1 (ROS1) inhibitors. A ROS1 inhibitor may be administered or formulated in combination with a RAS(ON) inhibitor and/or any additional therapeutic agent described herein. ROS1 is a receptor tyrosine kinase that belongs to the insulin receptor family and plays a role in various cellular processes, including cell growth, differentiation, survival, and migration. In some embodiments, a ROS1 inhibitor is one or more of taletrectinib, DS-6051b, TPX-0131, GZD824, and PF-06463922. In some embodiments, reference to the term ROS1 inhibitor includes any such ROS1 inhibitor disclosed in any one of the following patent applications: WO 2021098703, WO 2020024825, and US2017079972, each of which is incorporated herein by reference in its entirety, including the compound structures disclosed therein which are specifically incorporated herein by reference.

viii) PDGFR Inhibitors

In some embodiments, compositions and methods described herein may include a RAS(ON) inhibitor (e.g., RMC-6236, RMC-6291 and/or RMC-9805) in combination with one or more platelet-derived growth factor receptor (PDGFR) inhibitors. A PDGFR inhibitor may be administered or formulated in combination with a RAS(ON) inhibitor and/or any additional therapeutic agent described herein. PDGFR is a family of receptor tyrosine kinases that consists of two members, PDGFRa and PDGFRB. They are activated by binding to their ligands, such as platelet-derived growth factor (PDGF), which leads to the activation of downstream signaling pathways that promote cell growth, proliferation, and survival. In some embodiments, a PDGFR inhibitor is one or more of CP-673451, imatinib, nintedanib (ofev), sunitinib (sutent), pazopanib (votrient), regorafenib (stivarga), and dasatinib (sprycel).

### ix) FGF Inhibitors

In some embodiments, compositions and methods described herein may include a RAS(ON) inhibitor (e.g., RMC-6236, RMC-6291 and/or RMC-9805) in combination with fibroblast growth factor (FGF) inhibitors. An FGF inhibitor may be administered or formulated in combination with a RAS inhibitor and/or any additional therapeutic agent described herein. FGFRs are a family of receptor tyrosine kinases that consists of four members, FGFR1-4. FGFRs are activated by binding to their ligands, fibroblast growth factors (FGFs), which leads to the activation of downstream signaling pathways that promote cell growth, differentiation, and survival. In some embodiments, the FGFR inhibitor is an inhibitor of FGFR2. In some embodiments, the FGFR inhibitor is an inhibitor of FGFR4. In some embodiments, an FGFR inhibitor is one or more of futibatinib (TAK-659), erdafitinib (balversa), infigratinib (Truseltiq), Debio 1347, and rogaratinib (BAY 1163877). In some embodiments, reference to the term FGFR inhibitor includes any such FGFR inhibitor disclosed in any one of the following patent applications: WO 2022033472, WO 2022152274, WO 2022166469, WO 2022206939, WO 2021037219, WO 2021089005, WO 2021113462, WO 2020185532, WO 2019213544, WO 2020164603, WO 2019154364, WO 2019034076, WO 2019213506, WO 2019223766, WO 2018028438, WO 2018153373, WO 2018121650, WO 2018010514, WO 2017028816, WO 2017118438, WO 2016134320, WO 2015008844, WO 2014172644, WO 2014007951, WO 2013179033, WO 2013087578, WO 2012047699, CN 105906630, CN 115869315, CN 115141176, CN 115043832, and CN 115028634, each of which is incorporated herein by reference in its entirety. In some embodiments, the FGF pathway inhibitor targets an FGF ligand. Such FGF pathway inhibitors include FGF ligand traps and antibodies. Non-limiting examples include, FP-1039, an FGF ligand trap consisting of the extracellular domain of FGFR1 fused to the Fc portion of human IgG1, designed to sequester FGF ligands and inhibit FGF signaling, and MFGR1877S, a monoclonal antibody targeting FGF ligands, designed to block FGF-mediated signaling, including the compound structures disclosed therein which are specifically incorporated herein by reference.

### x) VEGF Inhibitors

In some embodiments, compositions and methods described herein may include a RAS(ON) inhibitor (e.g., RMC-6236, RMC-6291 and/or RMC-9805) in combination with one or more vascular endothelial growth factor (VEGF) signaling inhibitors. VEGF (vascular endothelial growth factor) signaling inhibitors are a class of drugs that target the signaling pathway mediated by VEGF and its receptors. VEGF plays a critical role in angiogenesis, the process of forming new blood vessels from existing ones, and it is overexpressed in many types of cancer, making it an attractive target for cancer therapy. A VEGF inhibitor may be administered or formulated in combination with a RAS(ON) inhibitor and/or any additional therapeutic agent described herein. In some embodiments, the VEGF inhibitor is an antibody or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto). In some embodiments, the VEGF inhibitor is one or more of bevacizumab, aflibercept, ramucirumab, sorafenib, sunitinib, and pazopanib.

### e) PI3K/mTOR Pathway Inhibitors

Compositions and methods described herein may include a RAS(ON) inhibitor (e.g., RMC-6236, RMC-6291 and/or RMC-9805) in combination with one or more inhibitors of the PI3K-AKT-TOR signaling pathway. The PI3K-AKT-mTOR signaling pathway is a critical intracellular pathway that regulates a wide range of cellular processes including cell growth, proliferation, metabolism, and survival. The pathway is initiated when growth factors, such as insulin or IGF-1, bind to cell surface receptors and activate phosphoinositide 3-kinase (PI3K). Activated PI3K then phosphorylates phosphatidylinositol 4,5-bisphosphate (PIP2) to produce phosphatidylinositol 3,4,5-trisphosphate (PIP3), which in turn activates AKT. Activated AKT then phosphorylates a variety of downstream targets including the tuberous sclerosis complex (TSC1/TSC2), leading to the activation of mTOR (mammalian target of rapamycin) complex 1 (mTORC1). Activated mTORC1 promotes protein synthesis and cell growth by phosphorylating key regulators of translation initiation such as S6 kinase (S6K) and eukaryotic initiation factor 4E-binding protein 1 (4E-BP1).

### i) PI3K Inhibitors

In some embodiments, compositions and methods described herein may include a RAS(ON) inhibitor (e.g., RMC-6236, RMC-6291 and/or RMC-9805) in combination with one or more PI3K inhibitors. A PI3K inhibitor may be administered or formulated in combination with a RAS(ON) inhibitor and/or any additional therapeutic agent described herein. PI3K inhibitors include, but are not limited to, wortmannin; 17-hydroxywortmannin analogs described in WO06/044453; 4-[2-(1H-Indazol-4-yl)-6-[4-(methylsulfonyl) piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-I-(4-((2-(2-aminopyrimidin-5-yl)-7-methyl-4-morpholinothieno[3,2-d]pyrimidin-6-yl)methyl) piperazin-1-yl)-2-hydroxypropan-1-one (described in WO08/070740); LY294002 (2-(4-morpholinyl)-8-phenyl-4H-I-benzopyran-4-one (available from Axon Medchem); PI 103 hydrochloride (3-[4-(4-morpholinylpyrido-[3′,2′: 4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[I-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136. In some embodiments, the PI3K inhibitor is alpelisib or copanlisib.

### ii) AKT Inhibitors

In some embodiments, compositions and methods described herein may include a RAS(ON) inhibitor (e.g., RMC-6236, RMC-6291 and/or RMC-9805) in combination with one or more AKT inhibitors. An AKT inhibitor may be administered or formulated in combination with a RAS(ON) inhibitor and/or any additional therapeutic agent described herein. AKT inhibitors include, but are not limited to, ipatasertib, GSK-2141795, Akt-1-1 (inhibits Aktl) (Barnett et al., Biochem. J. 2005, 385 (Pt. 2): 399-408); Akt-1-1,2 (inhibits Akl and 2) (Barnett et al., Biochem. J. 2005, 385 (Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134 (12 Suppl): 3493S-3498S); perifosine (e.g., interferes with Akt membrane localization; Dasmahapatra et al. Clin. Cancer Res. 2004, 10 (15): 5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9). The PI3K/AKT inhibitor may include, but is not limited to, one or more PI3K/AKT inhibitors described in Cancers (Basel) 2015 September; 7 (3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; and GSK2126458.

iii) mTOR Inhibitors

In some embodiments, compositions and methods described herein may include a RAS(ON) inhibitor (e.g., RMC-6236, RMC-6291 and/or RMC-9805) in combination with one or more mTOR inhibitors. A mTOR inhibitor may be administered or formulated in combination with a RAS(ON) inhibitor and/or any additional therapeutic agent described herein. mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl) rapamycin; 40-[3-hydroxy (hydroxymethyl)methylpropanoate]-rapamycin (also known as CC1779); 40-epi-(tetrazolyt)-rapamycin (also called ABT578); 32-deoxorapamycin; 16-pentynyloxy-32 (S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

### iv) MNK Inhibitors

In some embodiments, compositions and methods described herein may include a RAS(ON) inhibitor (e.g., RMC-6236, RMC-6291 and/or RMC-9805) in combination with one or more mitogen-activated protein kinase-interacting kinase (MNK) inhibitors. A MNK inhibitor may be administered or formulated in combination with a RAS(ON) inhibitor and/or any additional therapeutic agent described herein. MNK proteins are activated downstream of the mitogen-activated protein kinase (MAPK) signaling pathway, which plays a critical role in the regulation of cellular proliferation, differentiation, and survival. MNKs phosphorylate eIF4E, a key component of the eukaryotic translation initiation complex, which enhances the translation of specific mRNAs, including those encoding proteins involved in cell cycle regulation and oncogenesis. In some embodiments, a MNK inhibitor is one or more tomivosertib (eFT508), CGP57380, and SEL201. In some embodiments, reference to the term MNK inhibitor includes any such MNK inhibitor disclosed in any one of the following patent applications: WO 2021098691, WO 2020108619, WO 2020086713, WO 2018152117, WO 2018228275, WO 2015200481, and CN115583942, each of which is incorporated herein by reference in its entirety, including the compound structures disclosed therein which are specifically incorporated herein by reference.

v) eIF4 inhibitors

In some embodiments, compositions and methods described herein may include a RAS(ON) inhibitor (e.g., RMC-6236, RMC-6291 and/or RMC-9805) in combination with one or more eukaryotic initiation factor 4A (eIF4A) inhibitors. An eIF4A inhibitor may be administered or formulated in combination with a RAS(ON) inhibitor and/or any additional therapeutic agent described herein. eIF4A is a critical component of the eukaryotic translation initiation complex, where it functions as an RNA helicase to unwind the secondary structure of mRNA and facilitate ribosome binding. eIF4A is required for the translation of many cancer-associated genes, making it an attractive therapeutic target for cancer treatment. In some embodiments, an eIF4A inhibitor is one or more zotatifin (eFT226), silvestrol, pateamine A, and rocaglates. In some embodiments, reference to the term eIF4A inhibitor includes any such eIF4A inhibitor disclosed in any one of the following patent applications: WO 2023034813, WO 2021195128, and WO 2017091585, each of which is incorporated herein by reference in its entirety, including the compound structures disclosed therein which are specifically incorporated herein by reference.

In some embodiments, compositions and methods described herein may include one or more eukaryotic initiation factor 4G (eIF4G) inhibitors. An eIF4G inhibitor may be administered or formulated in combination with a RAS(ON) inhibitor and/or any additional therapeutic agent described herein. eIF4G family includes several proteins that are involved in the initiation of protein translation. eIF4G serves as a scaffold for other proteins, including eIF4E and eIF4A, to form the eIF4F complex, which is responsible for binding to the 5′ cap of mRNA and unwinding the secondary structure of the mRNA to allow ribosomal scanning and translation initiation. In some embodiments, an eIF4G inhibitor is one or more pateamine A, and hippuristanol.

### f) DNA Damage Response Inhibitors

Compositions and methods described herein may include a RAS(ON) inhibitor (e.g., RMC-6236, RMC-6291 and/or RMC-9805) in combination with one or more DNA damage response (DDR) inhibitors. The DDR pathway is a critical cellular pathway that is activated in response to DNA damage and is essential for maintaining genomic stability, thereby preventing the development of cancer. However, cancer cells often have defects in the DDR pathway, which makes them more sensitive to DDR inhibitors. DDR inhibitors have shown promise in preclinical studies as potential cancer therapeutics, particularly in combination with other agents.

### i) Wee1 Inhibitors

In some embodiments, compositions and methods described herein may include a RAS(ON) inhibitor (e.g., RMC-6236, RMC-6291 and/or RMC-9805) in combination with one or more Wee1 inhibitors. Wee1 is a kinase that plays a critical role in regulating the cell cycle by inhibiting the activity of cyclin-dependent kinases (CDKs) and preventing the progression of cells through the G2/M checkpoint. Wee1 is overexpressed in several cancer types and has been implicated in tumor growth and survival. In some embodiments, a Wee1 inhibitor is one or more of imp7068, adavosertib, or ZNL-02-096. In some embodiments, reference to the term Wee1 inhibitor includes any such Wee1 inhibitor disclosed in any one of the following patent applications: WO 2022011391, WO 2022247641, WO 2021043152, WO 2020221358, WO 2020083404, WO 2020192581, WO 2019085933, WO 2018133829, WO 2015115355, WO 2015183776, WO 2014085216, and CN 114831993, each of which is incorporated herein by reference in its entirety, including the compound structures disclosed therein which are specifically incorporated herein by reference.

### ii) CHK Inhibitors

In some embodiments, compositions and methods described herein may include a RAS(ON) inhibitor (e.g., RMC-6236, RMC-6291 and/or RMC-9805) in combination with one or more checkpoint kinase (CHK) inhibitors. A CHK inhibitor may be administered or formulated in combination with a RAS(ON) inhibitor and/or any additional therapeutic agent described herein. CHK1 kinase is a critical regulator of the cell cycle and the DNA damage response pathway. In some embodiments, the CHK inhibitor is a CHK1 inhibitor. In some embodiments, a CHK inhibitor is a CHK2 inhibitor. In some embodiments, a CHK1 inhibitor is one or more rabusertib, LY2606368, GDC-0575, and MK-8776. In some embodiments, reference to the term CHK1 inhibitor includes any such CHK1 inhibitor disclosed in any one of the following patent applications: WO 2021113661, WO 2021104461, WO 2019012030, WO 2010118390, WO 2008067027, WO 2002070494, and TW202126818, each of which is incorporated herein by reference in its entirety, including the compound structures disclosed therein which are specifically incorporated herein by reference.

iii) ATM Inhibitors

In some embodiments, compositions and methods described herein may include a RAS(ON) inhibitor (e.g., RMC-6236, RMC-6291 and/or RMC-9805) in combination with one or more ataxia telangiectasia mutated (ATM) inhibitors. An ATM inhibitor may be administered or formulated in combination with a RAS(ON) inhibitor and/or any additional therapeutic agent described herein. ATM plays a role in regulating the replication stress response and maintaining genomic stability. In some embodiments, an ATM inhibitor is one or more M4076, AZD0156, KU-60019, and VE-821. In some embodiments, reference to the term ATM inhibitor includes any such ATM inhibitor disclosed in any one of the following patent applications: WO 2021197339, WO 2021098734, WO 2021260580, WO 2007026157, WO 2006085067, and US2016113935, each of which is incorporated herein by reference in its entirety, including the compound structures disclosed therein which are specifically incorporated herein by reference.

### iv) ATR Inhibitors

In some embodiments, compositions and methods described herein may include a RAS(ON) inhibitor (e.g., RMC-6236, RMC-6291 and/or RMC-9805) in combination with one or more ataxia telangiectasia and Rad3-related (ATR) inhibitors. An ATR inhibitor may be administered or formulated in combination with a RAS(ON) inhibitor and/or any additional therapeutic agent described herein. In some embodiments, an ATR inhibitor is one or more ceralaertib, VE-821, RP-350, AZ20, VX-970, abd110, VX-803, and BAY 1895344. In some embodiments, reference to the term ATR inhibitor includes any such ATR inhibitor disclosed in any one of the following patent applications: WO 2023016529, WO 2022237875, WO 2022268025, WO 2021012049, WO 2021023272, WO 2021260579, WO 2021228758, WO 2019050889, WO 2019154365, WO 2019133711, WO 2017059357, WO 2013049859, WO 2007046426, WO 2007015632, and CN113797341, each of which is incorporated herein by reference in its entirety, including the compound structures disclosed therein which are specifically incorporated herein by reference.

### v) PARP Inhibitors

In some embodiments, compositions and methods described herein may include a RAS(ON) inhibitor (e.g., RMC-6236, RMC-6291 and/or RMC-9805) in combination with one or more Poly (ADP-ribose) polymerase (PARP) inhibitors. There are 17 PARP (aka tankyrase) family members that have been identified. PARP enzymes play a critical role in DNA damage repair, particularly in the repair of single-strand DNA breaks. PARP inhibitors block the activity of PARP enzymes, leading to the accumulation of DNA damage and ultimately cell death. In some embodiments, a PARP inhibitor is one or more Olaparib, rucaparib, niraparib, and veliparib (ABT-888). In some embodiments, reference to the term PARP inhibitor includes any such PARP inhibitor disclosed in any one of the following patent applications: WO 2023051812, WO 2023051807, WO 2023051716, WO 2023278592, WO 2022228387, WO 2022022664, WO 2022000946, WO 2022222921, WO 2021163530, WO 2020122034, WO 2020239097, WO 2020142583, WO 2020156577, WO 2020098774, WO 2020196712, WO 2019200382, WO 2018125961, WO 2018205938, WO 2018192576, WO 2018218025, WO 2017032289, WO 2017177838, WO 2017029601, WO 2017088723, WO 2016155655, WO 2015154630, WO 2013097225, WO 2012130166, WO 2011006794, WO 2009046205, WO 2009063244, WO 2008084261, WO 2007138351, WO 2006110816, WO 2005053662, WO 2005012524, CN113698356, CN 113603647, CN 115073544, CN 108938634, CN 104887680, CN 110343088, CN108976236, and CN 107629071, each of which is incorporated herein by reference in its entirety, including the compound structures disclosed therein which are specifically incorporated herein by reference.

### vi) DNA-PK Inhibitors

In some embodiments, compositions and methods described herein may include a RAS(ON) inhibitor (e.g., RMC-6236, RMC-6291 and/or RMC-9805) in combination with one or more DNA-dependent protein kinase (DNA-PK) inhibitors. DNA-dependent protein kinase (DNA-PK) is a serine/threonine protein kinase that plays a crucial role in DNA repair and maintenance of genome stability. In some embodiments, a DNA-PK inhibitor is one or more NU7441, AZD7648, VX-984, M3814, and CC-115. In some embodiments, reference to the term DNA-PK inhibitor includes any such DNA-PK inhibitor disclosed in any one of the following patent applications: WO 2022187965, WO 2021197159, WO 2021260583, WO 2021204111, WO 2021104277, WO 2021098813, WO 2021022078, WO 2020259613, WO 2019143678, WO 2019143675, WO 2019201283, WO 2015058031, WO 2014159690, WO 2012028233, WO 2009010761, WO 2006032869, WO 2006109084, CN 112574179, CN 112300132, and CN 112300126, each of which is incorporated herein by reference in its entirety, including the compound structures disclosed therein which are specifically incorporated herein by reference.

### g) Cell Cycle Inhibitors

Compositions and methods described herein may include a RAS(ON) inhibitor (e.g., RMC-6236, RMC-6291 and/or RMC-9805) in combination with one or more cell cycle inhibitors. Cell cycle inhibitors target specific proteins involved in regulating the cell cycle, which is the process by which a cell divides and replicates its DNA. Non-limiting examples cell cycle proteins include cyclin-dependent kinase (CDK), aurora kinase, and polo-like kinase (PLK). CDKs are a family of kinases that are involved in regulating the cell cycle. CDK inhibitors block the activity of these kinases, leading to cell cycle arrest and/or apoptosis. Aurora kinases are a family of serine/threonine kinases that play a critical role in regulating mitosis. Aurora kinase inhibitors block the activity of these kinases, leading to mitotic arrest and cell death. PLKs are a family of serine/threonine kinases that are involved in regulating multiple stages of the cell cycle. PLK inhibitors block the activity of these kinases, leading to cell cycle arrest and/or apoptosis.

### i) CDK Inhibitors

In some embodiments, compositions and methods described herein may include a RAS(ON) inhibitor (e.g., RMC-6236, RMC-6291 and/or RMC-9805) in combination with one or more CDK inhibitors. A CDK inhibitor may be administered or formulated in combination with a RAS(ON) inhibitor and/or any additional therapeutic agent described herein. Cyclin-dependent kinases are a family of protein kinases that regulate cell division and proliferation. Cell cycle progression is controlled by cyclins and their associated cyclin-dependent kinases, such as CDK1, CDK2, CDK3, CDK4 and CDK6, while other CDKs such as CDK7, CDK8 and CDK9 are critical to transcription. CDK binding to cyclins forms heterodimeric complexes that phosphorylate their substrates on serine and threonine residues, which in turn initiates events required for cell-cycle transcription and progression. In some embodiments, a CDK inhibitor is a CDK2 inhibitor. In some embodiments, a CDK inhibitor is a CDK4/6 inhibitor. In some embodiments, a CDK inhibitor is a CDK7 inhibitor. In some embodiments, a CDK inhibitor is a CDK9 inhibitor. In some embodiments, a CDK inhibitor is one or more palbociclib, ribociclib, abemaciclib, and trilaciclib. In some embodiments, a CDK inhibitor is one or more of tagtociclib (PF-07104091), seliciclib, voruciclib P1446A-05, BLU-222, dinaciclib, AT-7519, RGB286638, and AZD4573.

In some embodiments, reference to the term CDK inhibitor includes any such CDK inhibitor disclosed in any one of the following patent applications: WO 2022166793, WO 2022187611, WO 2022130304, WO 2021227906, WO 2021057867, WO 2020207260, WO 2020138370, WO 2020125513, WO 2020148635, WO 2020215156, WO 2020052627, WO 2017177837, WO 2017162215, WO 2017177836, WO 2016193939, WO 2016014904, WO 2016015598, WO 2016015605, WO 2015181737, WO 2012061156 A1, WO 2012038411, WO 2010020675, WO 2010125004, WO 2007139732, WO 2006024945, CN 114478529, CN 108794496, CN 105294737, CN107652284, KR 20180106188, and US 2017152269, each of which is incorporated herein by reference in its entirety, including the compound structures disclosed therein which are specifically incorporated herein by reference.

### ii) Aurora Kinase Inhibitors

In some embodiments, compositions and methods described herein may include a RAS(ON) inhibitor (e.g., RMC-6236, RMC-6291 and/or RMC-9805) in combination with one or more aurora kinase inhibitors. An aurora kinase inhibitor may be administered or formulated in combination with a RAS(ON) inhibitor and/or any additional therapeutic agent described herein. Aurora kinases are a family of serine/threonine kinases that play a critical role in regulating cell division and maintaining genomic stability. The Aurora kinase family consists of three members: Aurora A, Aurora B, and Aurora C. In some embodiments, an aurora kinase inhibitor is one or more palbociclib, ribociclib, and abemaciclib. In some embodiments, an aurora kinase inhibitor is one or more of alisertib, danusertib, barasertib, and MLN8237. In some embodiments, reference to the term aurora kinase inhibitor includes any such aurora kinase inhibitor disclosed in any one of the following patent applications: WO 2021110009, WO 2021008338, WO 2020112514, WO 2019129234, WO 2016077161, WO 2013143466, WO 2011103089, WO 2010081881, WO 2010133794, WO 2009134658, WO 2008001886, WO 2007095124, WO 2007003596, WO 2006129064, CN 114276227, CN 108078991, CN 106543155, CN 104211692, and CN 104098551, each of which is incorporated herein by reference in its entirety, including the compound structures disclosed therein which are specifically incorporated herein by reference.

iii) PLK Inhibitors

In some embodiments, compositions and methods described herein may include a RAS(ON) inhibitor (e.g., RMC-6236, RMC-6291 and/or RMC-9805) in combination with one or more polo-like kinase (PLK) inhibitors. A PLK inhibitor may be administered or formulated in combination with a RAS(ON) inhibitor and/or any additional therapeutic agent described herein. PLKs are a family of serine/threonine kinases that play a crucial role in regulating cell division, DNA damage response, mitotic progression, and consists of four members: PLK1, PLK2, PLK3, and PLK4. In some embodiments, a PLK inhibitor is one or more of volasertib, onvansertib, BI 2536, and GSK461364. In some embodiments, reference to the term PLK inhibitor includes any such PLK inhibitor disclosed in any one of the following patent applications: WO 2011012534 A1, WO 2010065134, WO 2009130453, WO 2009042806, WO 2004043936, WO 2007030361, WO 2006021547, CN 115804777, and EP 2325185, each of which is incorporated herein by reference in its entirety, including the compound structures disclosed therein which are specifically incorporated herein by reference.

### iv) Kinesin Superfamily of Microtubule Motor Protein Inhibitors

In some embodiments, compositions and methods described herein may include a RAS(ON) inhibitor (e.g., RMC-6236, RMC-6291 and/or RMC-9805) in combination with one or more Kinesin spindle protein (KSP) inhibitors. In some embodiments, compositions described herein may include one or more Kinesin family (KIF) inhibitors. In some embodiments, a KSP inhibitor may be administered or formulated in combination with a RAS(ON) inhibitor and/or any additional therapeutic agent described herein. KSP and KIF are a subset of the kinesin superfamily of microtubule motor proteins. KSP, also known as Eg5, is a member of the kinesin superfamily of motor proteins that plays a critical role in mitotic spindle formation and cell division. KSP inhibitors selectively target rapidly dividing cancer cells by disrupting spindle formation and inducing mitotic arrest. In some embodiments, a KSP inhibitor is one or more of SB743921, monastrol, S-Trityl-L-cysteine (STLC), and filanesib (ARRY-520). In some embodiments, a KIF inhibitor is an inhibitor of a Kinesin-8 family microtubule motor protein. In some embodiments, the kinesin-8 family protein is KIF18A. In some embodiments, a KIF inhibitor is one or more of AMG650, BTB-1, K03861, and SJ000291942. In some embodiments, reference to the term kinesin superfamily of microtubule motor protein inhibitor includes any such kinesin superfamily of microtubule motor protein inhibitor disclosed in any one of the following patent applications: WO 2015114854, WO 2015114855, WO 2010084186, WO 2006101761, WO 2006110390, WO 2006044825, WO 2006078574, WO 2005060654, WO 2004092147, WO 2004037171, WO 2004058700, WO 2003050064, WO 2003105855, WO 2022037665, WO 2018114804, WO 2017162663, WO 2016207089, WO 2012073375, JP 2014162787, JP 2019189590, JP2013166713, and KR 20220145566, each of which is incorporated herein by reference in its entirety, including the compound structures disclosed therein which are specifically incorporated herein by reference.

### v) DYRK1 Inhibitors

In some embodiments, compositions and methods described herein may include a RAS(ON) inhibitor (e.g., RMC-6236, RMC-6291 and/or RMC-9805) in combination with one or more Dual-specificity tyrosine phosphorylation-regulated kinase 1 (DYRK1) inhibitors. A DYRK1 inhibitor may be administered or formulated in combination with a RAS(ON) inhibitor and/or any additional therapeutic agent described herein. DYRK1 is a member of the DYRK (dual-specificity tyrosine phosphorylation-regulated kinase) family of protein kinases. It plays essential roles in various cellular processes, including cell cycle regulation, neuronal development, and transcriptional control. In some embodiments, a DYRK1 inhibitor is one or more of harmine, INDY, D4476, and AZ191. In some embodiments, reference to the term DYRK1 inhibitor includes any such DYRK1 inhibitor disclosed in any one of the following patent applications: WO 2023277331 A1, WO 2023140846 A1, WO 2017181087 A1, each of which is incorporated herein by reference in its entirety, including the compound structures disclosed therein which are specifically incorporated herein by reference.

### h) Anti-Apoptotic Protein Inhibitors

Compositions and methods described herein may include a RAS(ON) inhibitor (e.g., RMC-6236, RMC-6291 and/or RMC-9805) in combination with one or more anti-apoptotic protein inhibitors. In some embodiments, an anti-apoptotic protein inhibitor may be administered or formulated in combination with a RAS(ON) inhibitor and/or any additional therapeutic agent described herein. Anti-apoptotic inhibitors target proteins that play a role in preventing apoptosis, a form of programmed cell death. Apoptosis is a critical mechanism for eliminating damaged or unwanted cells. Anti-apoptotic proteins are a family of proteins that inhibit the apoptotic pathway, thereby preventing cell death. There are several known classes of anti-apoptotic inhibitors, including Bcl-2 inhibitors, XIAP inhibitors, survivin inhibitors, Mcl-1 inhibitors, and FLIP inhibitors. These inhibitors work by binding to specific anti-apoptotic proteins and preventing their activity, thereby promoting cell death in cancer cells. In some embodiments, compositions described herein may include one or more anti-apoptotic protein inhibitors. An anti-apoptotic protein inhibitor may be administered or formulated in combination with a RAS(ON) inhibitor and/or any additional therapeutic agent described herein. In some embodiments, the anti-apoptotic protein inhibitor includes a MCL-1 inhibitor. Non-limiting examples of MCL-1 inhibitors include, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263. In some embodiments, the anti-apoptotic protein inhibitor includes a BCL protein inhibitor. Examples of BCL protein inhibitors include but are not limited to Venetoclax (Venclexta), Navitoclax (ABT-263), A-1331852, S63845, and AT-101.

### i) Autophagy Inhibitors

Compositions and methods described herein may include a RAS(ON) inhibitor (e.g., RMC-6236, RMC-6291 and/or RMC-9805) in combination with one or more autophagy inhibitors. In some embodiments, an autophagy inhibitor may be administered or formulated in combination with a RAS(ON) inhibitor and/or any additional therapeutic agent described herein. Autophagy inhibitors include, but are not limited to chloroquine, 3-methyladenine, hydroxychloroquine (Plaquenil™), spautin-1, SAR405, bafilomycin A1, 5-amino-4-imidazole carboxamide riboside (AICAR), okadaic acid, autophagy-suppressive algal toxins which inhibit protein phosphatases of type 2A or type 1, analogues of CAMP, and drugs which elevate CAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

### a) ULK Inhibitors

In some embodiments, compositions and methods described herein may include a RAS(ON) inhibitor (e.g., RMC-6236, RMC-6291 and/or RMC-9805) in combination with one or more Unc-51-like kinase (ULK) inhibitors. An ULK inhibitor may be administered or formulated in combination with a RAS(ON) inhibitor and/or any additional therapeutic agent described herein. In some embodiments, a ULK inhibitor is a ULK1/2 inhibitor. In some embodiments, an ULK inhibitor is one or more of ULK-101, MRT68921, SBI-0206965, MRT67307, MRT68920, MRT68922, MRT199665, LY3009120, and Dorsomorphin.

### b) VPS Inhibitors

In some embodiments, compositions and methods described herein may include a RAS(ON) inhibitor (e.g., RMC-6236, RMC-6291 and/or RMC-9805) in combination with one or more Vacuolar protein sorting protein (VPS) inhibitors. A VPS inhibitor may be administered or formulated in combination with a RAS(ON) inhibitor and/or any additional therapeutic agent described herein. VPS (proteins are a family of proteins that play a critical role in the process of autophagy by regulating the formation and function of autophagosomes, structures that engulf and transport cellular components to lysosomes for degradation. Dysregulation of VPS proteins has been implicated in various diseases, including cancer, neurodegenerative disorders, and infectious diseases. In some embodiments, a VPS inhibitor is a VPS34 inhibitor. In some embodiments, a VPS inhibitor is one or more of PIK-III, VPS34-IN1, SAR405, Spautin-1, and NSC185058.

### c) Macropinocytosis Inhibitors

In some embodiments, compositions and methods described herein may include a RAS(ON) inhibitor (e.g., RMC-6236, RMC-6291 and/or RMC-9805) in combination with one or more macropinocytosis inhibitors. A macropinocytosis inhibitor may be administered or formulated in combination with a RAS(ON) inhibitor and/or any additional therapeutic agent described herein. Macropinocytosis inhibitors are compounds that can block or reduce the process of macropinocytosis. In some embodiments, a macropinocytosis inhibitor is one or more of EIPA (ethylisopropylamiloride), Wortmannin, Amiloride, Apilimod, Dyngo-4a, and Latrunculin B.

### j) WNT/β-Catenin Pathway Inhibitors

Compositions and methods described herein may include a RAS(ON) inhibitor (e.g., RMC-6236, RMC-6291 and/or RMC-9805) in combination with one or more WNT/beta-catenin pathway inhibitors. In some embodiments, a WNT/beta-catenin pathway inhibitor may be administered or formulated in combination with a RAS(ON) inhibitor and/or any additional therapeutic agent described herein. The WNT/beta-catenin pathway is an important signaling pathway that plays a crucial role in development, tissue homeostasis, and disease. Dysregulation of this pathway has been implicated in various cancers, making it an attractive target for cancer therapy. WNT/beta-catenin pathway inhibitors target various components of the pathway, including WNT ligands, receptors, and downstream effectors.

### i) β-Catenin Inhibitors

In some embodiments, compositions and methods described herein may include one or more β-catenin inhibitors. A β-catenin inhibitor may be administered or formulated in combination with a RAS inhibitor therapy and/or any additional therapeutic agent described herein. Beta-catenin is a protein that plays an important role in the WNT signaling pathway, which regulates various cellular processes including cell proliferation, differentiation, and migration. In normal cells, β-catenin levels are tightly regulated by a destruction complex, which marks beta-catenin for degradation. However, in many cancer cells, the destruction complex is impaired, leading to the accumulation of beta-catenin in the nucleus and the activation of target genes involved in tumor growth and metastasis. In some embodiments, a WNT/β-catenin inhibitor is one or more of FOG-001, OMP-131R10, Foxy-5, LGK974, RXC004, ETC-159, OMP-54F28, Niclosamide, OMP-18R5, OTSA-101, BNC101, DKN-01, Sulindac, Pyrvinium, E7449, BC2059, PRI-724, SM08502, IWP1, IWP2, IWP3, IWP4, IWP12, IWP L6, C59, GNF-6231, GNF-1331, DK-520, DK-419, IgG-2919, Fz7-21, RHPD-P1, SRI37892, 1094-0205, 2124-0331, 3235-0367, NSC36784, NSC654259, IgG-2919, Salinomycin, BMD4702, 3289-8625, J01-017a, FJ9, KY-02061, KY-02327, NSC668036, Peptide Pen-N3, SSTC3, CCT031374, TCS 183, XAV939, AZ1366, G007-LK, MSC2504877, G244-LM, IWR-1, JW74, JW55, K-756, NVP-TNKS656, MN-64, RK-287107, WIKI4, KY1220, KYA1797K, MSAB, PKF115-584, CGP049090, AV-65, PNU-74654, Windorphen, IQ-1 tegavivant, foscenvivant, PNPB-29, ZW4864, SAH-BCL9, Carnosic acid, xStAx-VHL, NRX-252114, Septuximab vedotin, PF-06647020, LGR5-mc-vc-PAB-MMAE, LGR5-NMS818, CWP232291, PRI-724 (also known as ICG-001), C-82, and BC2059. In some embodiments, reference to the term β-catenin inhibitor includes any such β-catenin inhibitor disclosed in any one of the following patent applications: CN 104388427 and CN 103830211, each of which is incorporated herein by reference in its entirety, including the compound structures disclosed therein which are specifically incorporated herein by reference.

### ii) PORCN Inhibitors

In some embodiments, compositions and methods described herein may include a RAS(ON) inhibitor (e.g., RMC-6236, RMC-6291 and/or RMC-9805) in combination with one or more Porcupine (PORCN) inhibitors. A PORCN inhibitor may be administered or formulated in combination with a RAS(ON) inhibitor and/or any additional therapeutic agent described herein. PORCN is a membrane-bound O-acyltransferase enzyme that plays a critical role in the WNT signaling pathway by mediating the palmitoylation of WNT ligands. This palmitoylation is essential for the secretion and signaling activity of WMT proteins. Inhibition of PORCN leads to reduced WNT signaling activity. In some embodiments, a PORCN inhibitor is one or more of LGK974 (WNT974), ETC-1922159, CGX1321, and CWP232291.

iii) GSK3 Inhibitors

In some embodiments, compositions and methods described herein may include a RAS(ON) inhibitor (e.g., RMC-6236, RMC-6291 and/or RMC-9805) in combination with one or more Glycogen synthase kinase (GSK3) inhibitors. A GSK3 inhibitor may be administered or formulated in combination with a RAS(ON) inhibitor and/or any additional therapeutic agent described herein. The GSK3 family consists of two closely related serine/threonine kinases: GSK3a and GSK3B. These kinases are involved in numerous cellular processes, including glycogen metabolism, cell cycle regulation, and Wnt signaling. GSK inhibitors have been investigated as potential therapeutics for various diseases, including cancer, diabetes, Alzheimer's disease, and bipolar disorder. In some embodiments, a GSK3 inhibitor is one or more of Tideglusib, laduviglusib, LiCl (Lithium chloride), CHIR99021, SB216763, AZD1080, and LY2090314. In some embodiments, reference to the term GSK3 inhibitor includes any such GSK3 inhibitor disclosed in any one of the following patent applications: WO 2017153834, WO 2014059383, WO 2010012398, WO 2009017455, WO 2003037891, CN 107151235, and CN 102258783, each of which is incorporated herein by reference in its entirety, including the compound structures disclosed therein which are specifically incorporated herein by reference.

### iv) CLK Inhibitors

In some embodiments, compositions and methods described herein may include a RAS(ON) inhibitor (e.g., RMC-6236, RMC-6291 and/or RMC-9805) in combination with one or more Cdc2-like kinase (CLK) inhibitors. A CLK inhibitor may be administered or formulated in combination with a RAS(ON) inhibitor and/or any additional therapeutic agent described herein. LKs (Cdc2-like kinases) are a family of serine/threonine kinases that play a crucial role in pre-mRNA splicing, specifically in the regulation of alternative splicing. There are four members of the CLK family: CLK1, CLK2, CLK3, and CLK4. The CLK family of kinases have been shown to be involved in several diseases, including cancer, neurodegenerative disorders, and viral infections. In some embodiments, a CLK inhibitor is a CLK 2 inhibitor. In some embodiments, a CLK2 inhibitor is one or more of Lorecivivint, SM08502, SM04690, TG003, KH-CB19, Cmpd-1, T3.5, and CX-4945. In some embodiments, reference to the term CLK inhibitor includes any such CLK inhibitor disclosed in WO 2020006115, which is incorporated herein by reference in its entirety, including the compound structures disclosed therein which are specifically incorporated herein by reference.

### k) JAK/STAT Pathway Inhibitors

Compositions and methods described herein may include a RAS(ON) inhibitor (e.g., RMC-6236, RMC-6291 and/or RMC-9805) in combination with one or more JAK/STAT pathway inhibitors. In some embodiments, a JAK/STAT pathway inhibitor may be administered or formulated in combination with a RAS inhibitor and/or any additional therapeutic agent described herein. The Janus kinase/signal transducer and activator of transcription (JAK/STAT) pathway is a signaling pathway involved in many cellular processes, including immune response, cell growth, and differentiation. Dysregulation of this pathway has been linked to various diseases, including inflammatory disorders, cancer, and autoimmune diseases. Inhibitors of the JAK/STAT pathway can be used for the treatment of these diseases. In some embodiments, a JAK/STAT pathway inhibitor is an inhibitor of JAK1, JAK2 and/or JAK3. In some embodiments, a JAK inhibitor is one or more of Ruxolitinib (Jakafi®), Pacritinib, Fedratinib, Tofacitinib (Xeljanz®), Abrocitinib, Filgotinib, Oclacitinib, Peficitinib, Upadacitinib, Deucravacitinib, Delgocitinib, and Baricitinib (Olumiant®). In some embodiments, reference to the term JAK inhibitor includes any such JAK inhibitor disclosed in any one of the following patent applications: WO 2023011301, WO 2023201044, WO 2022143629, WO 2022251434, WO 2022067106, WO 2022033551, WO 2021244323, WO 2021238817, WO 2021238818, WO 2021178991, WO 2021136345, WO 2021190647, WO 2020219639, WO 2020182159, WO 2020155931, WO 2020038457, WO 2020219524, WO 2020173400, WO 2018204233, WO 2018204238, WO 2018169875, WO 2018117152, WO 2017215630, WO 2016070697, WO 2016027195, CN 117815195, CN117815367, and CN 115969796, each of which is incorporated herein by reference in its entirety, including the compound structures disclosed therein which are specifically incorporated herein by reference.

In some embodiments, the JAK/STAT pathway inhibitor is a STAT inhibitor. In some embodiments, the STAT inhibitor is an inhibitor of STAT3 and/or STAT5. In some embodiments, the STAT inhibitor is a STAT3 degrader. In some embodiments, the STAT inhibitor is one or more of TTI-101, C-188-9, WP1066, VVD-130850, LLL12B, STA-21, SD-36, Stattic, S31-201, OPB-31121, and Napabucasin (BBI608). In some embodiments, reference to the term STAT inhibitor includes any such STAT inhibitor disclosed in any one of the following patent applications: WO 2024030628, WO 2023164680, WO 2023192960, WO 2023133336, WO2020206424, WO 2023107706, WO 2021150543, WO 2008151037, and CN 109288845, each of which is incorporated herein by reference in its entirety, including the compound structures disclosed therein which are specifically incorporated herein by reference.

### l) Epigenetic Modulators

Compositions and methods described herein may include a RAS(ON) inhibitor (e.g., RMC-6236, RMC-6291 and/or RMC-9805) in combination with one or more epigenetic modulators. Epigenetic modulators are a class of therapeutics that target enzymes responsible for modifying the structure and function of chromatin, the complex of DNA and proteins that make up chromosomes. These enzymes, including histone deacetylases (HDACs), histone methyltransferases (HMTs), and DNA methyltransferases (DNMTs), play critical roles in gene expression and regulation by modifying the packaging of DNA and affecting how it is read and transcribed. Epigenetic modulators work by altering the activity of these enzymes, either by inhibiting or enhancing their function, to regulate gene expression in specific ways. By targeting specific epigenetic modifications, such as acetylation, methylation, and DNA methylation, these therapies have the potential to treat a wide range of diseases, including cancer, inflammatory disorders, and neurological disorders.

### i) HDAC Inhibitors

In some embodiments, compositions and methods described herein may include a RAS(ON) inhibitor (e.g., RMC-6236, RMC-6291 and/or RMC-9805) in combination with one or more histone deacetylase (HDAC) inhibitors. A HDAC inhibitor may be administered or formulated in combination with a RAS(ON) inhibitor and/or any additional therapeutic agent described herein. There are several classes of HDACs, including class I, class IIa, class IIb, class III, and class IV. Class I HDACs are further divided into HDAC1, HDAC2, HDAC3, and HDAC8, while class IIa HDACs include HDAC4, HDAC5, HDAC7, and HDAC9. Class Ilb HDACs consist of HDAC6 and HDAC10, and class III HDACs are known as sirtuins. HDAC inhibitors can target different classes of HDACs, and their specific effects on gene expression can vary depending on which HDACs they target. In some embodiments, a HDAC inhibitor is one or more of Vorinostat (Zolinza), Romidepsin (Istodax), Belinostat (Beleodaq), Panobinostat (Farydak), Entinostat (MS-275), Valproic acid (Depakene), Trichostatin A (TSA), Sodium butyrate, and Mocetinostat (MGCD0103). Non-limiting examples of HDAC inhibitors include trichostatin, sodium butyrate, apicidan, suberoyl anilide hydroamic acid, vorinostat, LBH 589, romidepsin, ACY-1215, and Panobinostat. In some embodiments, reference to the term HDAC inhibitor includes any such HDAC inhibitor disclosed in any one of the following patent applications: WO 2022110958, WO 2021252628, WO 2019204550, WO 2018178060, WO 2016126724, WO 2014143666, WO 2013041480, and WO 2006120456, each of which is incorporated herein by reference in its entirety, including the compound structures disclosed therein which are specifically incorporated herein by reference.

### ii) BET Inhibitors

In some embodiments, compositions and methods described herein may include a RAS(ON) inhibitor (e.g., RMC-6236, RMC-6291 and/or RMC-9805) in combination with one or more bromodomain and extra-terminal protein (BET) inhibitors. A BET inhibitor may be administered or formulated in combination with a RAS(ON) inhibitor and/or any additional therapeutic agent described herein. BET (bromodomain and extra-terminal) proteins are a family of epigenetic reader proteins that recognize and bind to acetylated lysine residues on histones, leading to chromatin remodeling and gene expression regulation. There are four BET proteins in humans: BRD2, BRD3, BRD4, and BRDT. BET inhibitors specifically target the bromodomains of BET proteins, inhibiting their binding to acetylated lysine residues on histones and leading to alterations in gene expression. BET inhibitors are useful in the treatment of cancer and other diseases characterized by dysregulated gene expression. In some embodiments, a BET inhibitor is one or more of JQ1, I-BET762, OTX015, RVX-208, and CPI-0610. In some embodiments, reference to the term BET inhibitor includes any such BET inhibitor disclosed in any one of the following patent applications: WO 2022046682, WO 2022182857, WO 2021107657, WO 2021107656, WO 2020221006, WO 2020053660, WO 2018097977, WO 2017222977, WO 2017142881, WO 2015075665, WO 2015011084, and CN 113264930, each of which is incorporated herein by reference in its entirety, including the compound structures disclosed therein which are specifically incorporated herein by reference.

iii) EZH2 Inhibitors

In some embodiments, compositions and methods described herein may include a RAS(ON) inhibitor (e.g., RMC-6236, RMC-6291 and/or RMC-9805) in combination with one or more Enhancer of Zeste Homolog 2 (EZH2) inhibitors. A EZH2 inhibitor may be administered or formulated in combination with a RAS(ON) inhibitor and/or any additional therapeutic agent described herein. EZH2 is a histone-lysine N-methyltransferase that is a member of the Polycomb repressive complex 2 (PRC2) family. EZH2 plays a crucial role in gene expression regulation, specifically by catalyzing the trimethylation of histone H3 at lysine 27 (H3K27me3), leading to transcriptional repression of target genes. EZH2 has been found to be overexpressed in several types of cancers and is associated with tumor progression and poor prognosis. In some embodiments, an EZH2 inhibitor is one or more of Tazemetostat, GSK2816126, and CPI-1205 (lirametostat). In some embodiments, reference to the term EZH2 inhibitor includes any such EZH2 inhibitor disclosed in any one of the following patent applications: WO 2023030299, WO 2022179584, WO 2020224607, WO 2021243060, WO 2021086069, WO 2019206155, WO 2018133795, WO 2018137639, WO 2017184999, WO 2017218953, WO 2016201328, WO 2015195848, WO 2013155317, WO 2013138361, and CN 114621191, each of which is incorporated herein by reference in its entirety, including the compound structures disclosed therein which are specifically incorporated herein by reference.

### iv) Co-REST Inhibitors

In some embodiments, compositions and methods described herein may include a RAS(ON) inhibitor (e.g., RMC-6236, RMC-6291 and/or RMC-9805) in combination with one or more Co-REST inhibitors. A Co-REST inhibitor may be administered or formulated in combination with a RAS(ON) inhibitor and/or any additional therapeutic agent described herein. Co-REST is a transcriptional co-repressor protein that interacts with a variety of transcription factors to regulate gene expression. Co-REST acts by recruiting histone deacetylases (HDACs) to chromatin, leading to the repression of gene expression. Inhibition of Co-REST has been proposed as a potential therapeutic strategy for the treatment of various diseases, including neurodegenerative disorders and cancer. In some embodiments, a co-REST inhibitor is one or more of Nocodazole, NSC 1892, and Anacardic acid.

### v) EP300

In some embodiments, compositions and methods described herein may include a RAS(ON) inhibitor (e.g., RMC-6236, RMC-6291 and/or RMC-9805) in combination with one or more E1A-binding protein p300 (EP300) inhibitors. An EP300 inhibitor may be administered or formulated in combination with a RAS(ON) inhibitor and/or any additional therapeutic agent described herein. EP300 is a transcriptional co-activator involved in the regulation of numerous cellular processes, including chromatin remodeling, DNA damage response, and cell cycle progression. EP300 acts as a histone acetyltransferase, catalyzing the transfer of acetyl groups to lysine residues on histone proteins, which leads to changes in chromatin structure and gene expression. EP300 activity has been implicated in diseases, such as cancer, cardiovascular and neurological disorders. In some embodiments, an EP300 inhibitor is one or more of C646, A-485, NU9056, and L002. In some embodiments, reference to the term EP300 inhibitor includes any such EP300 inhibitor disclosed in any one of the following patent applications: WO 2021213521 and WO 2016044694, each of which is incorporated herein by reference in its entirety, including the compound structures disclosed therein which are specifically incorporated herein by reference.

### vi) LSD1

In some embodiments, compositions and methods described herein may include a RAS(ON) inhibitor (e.g., RMC-6236, RMC-6291 and/or RMC-9805) in combination with one or more Lysine-specific demethylase 1 (LSD1) inhibitors. A LSD1 inhibitor may be administered or formulated in combination with a RAS(ON) inhibitor and/or any additional therapeutic agent described herein. LSD1 is an enzyme that plays a crucial role in regulating gene expression through histone modification. It specifically removes the methyl group from lysine 4 on histone 3, leading to gene repression. Dysregulation of LSD1 has been associated with various diseases including cancer and neurodegenerative disorders. In some embodiments, a LSD1 inhibitor is one or more of GSK2879552, IMG-7289, ORY-1001, IMG-8419, SP-2577, CC-90011, HCl-2509, and INCB059872. In some embodiments, reference to the term LSD1 inhibitor includes any such LSD1 inhibitor disclosed in any one of the following patent applications: WO 2021095840, WO 2021175079, WO 2021058024, WO 2020047198, WO 2020052649, WO 2020015745, WO 2020052647, WO 2018137644, WO 2017184934, WO 2017027678, WO 2017116558, WO 2017149463, WO 2016161282, WO 2015123465, WO 2015123424, WO 2013057322, WO 2013057320, WO 2012135113, CN 114805261, CN 111072610 CN107174584, CN 110478352, CN 106432248, and CN 106045881, each of which is incorporated herein by reference in its entirety, including the compound structures disclosed therein which are specifically incorporated herein by reference.

vii) PRMT5

In some embodiments, compositions and methods described herein may include a RAS(ON) inhibitor (e.g., RMC-6236, RMC-6291 and/or RMC-9805) in combination with one or more Protein arginine methyltransferase 5 (PRMT5) inhibitors. A PRMT5 inhibitor may be administered or formulated in combination with a RAS(ON) inhibitor and/or any additional therapeutic agent described herein. PRMT5 is a member of the PRMT family, which catalyzes the transfer of a methyl group from S-adenosylmethionine (SAM) to the nitrogen atoms of arginine residues in target proteins. PRMT5 is involved in various biological processes, including gene expression regulation, signal transduction, and DNA repair. In some embodiments, a PRMT5 inhibitor is one or more of TNG908, TNG462, AMG193, GSK591, EPZ015666, TC-E 5003, and MS023. In some embodiments, reference to the term PRMT5 inhibitor includes any such PRMT5 inhibitor disclosed in any one of the following patent applications: WO 2023001133, WO 2022206964, WO 2022153161, WO 2021068953, WO 2021088992, WO 2020259478, WO 2020205660, WO 2020250123, WO 2020033288, WO 2019102494, WO 2019112719, WO 2019180631, WO 2018065365, WO 2017153186, WO 2017212385, WO 2017032840, WO 2016022605, WO2014100695, WO 2014145214, WO 2014100719, CN 111825656, CN 114558014, CN 11304554, and CN 112778275, each of which is incorporated herein by reference in its entirety, including the compound structures disclosed therein which are specifically incorporated herein by reference.

viii) MAT2A

In some embodiments, compositions and methods described herein may include a RAS(ON) inhibitor (e.g., RMC-6236, RMC-6291 and/or RMC-9805) in combination with one or more methionine adenosyltransferase 2A (MAT2A) inhibitors. A MAT2A inhibitor may be administered or formulated in combination with a RAS(ON) inhibitor and/or any additional therapeutic agent described herein. MAT2A is an enzyme that catalyzes the production of S-adenosylmethionine (SAM), which is an important cofactor in many biological processes, including DNA methylation, protein methylation, and polyamine synthesis. Elevated MAT2A expression has been associated with various cancers. In some embodiments, a MAT2A inhibitor is one or more of cycloleucine and 2-hydroxy-4-methylthiobutanoic acid. In some embodiments, reference to the term MAT2A inhibitor includes any such MAT2A inhibitor disclosed in any one of the following patent applications: WO 2022256808, WO 2022256806, WO 2019191470, and CN 115716831, each of which is incorporated herein by reference in its entirety, including the compound structures disclosed therein which are specifically incorporated herein by reference.

### ix) DOT1L

In some embodiments, compositions and methods described herein may include a RAS(ON) inhibitor (e.g., RMC-6236, RMC-6291 and/or RMC-9805) in combination with one or more Disruptor of Telomeric silencing 1-like (DOT1L) inhibitors. A DOT1L inhibitor may be administered or formulated in combination with a RAS(ON) inhibitor and/or any additional therapeutic agent described herein. DOT1L is a histone methyltransferase enzyme that catalyzes the methylation of lysine 79 on histone H₃. This modification is associated with transcriptional elongation and is important for the maintenance of gene expression programs. The DOT1L family includes enzymes that are involved in epigenetic regulation and transcriptional control, and their dysregulation has been linked to various diseases, including cancer. In some embodiments, a DOT1L inhibitor is one or more of EPZ-5676 (pinometostat) and EPZ-004777. In some embodiments, reference to the term DOT1L inhibitor includes any such DOT1L inhibitor disclosed in any one of the following patent applications: WO 2016090271, WO 2014100662, and CN 108997480, each of which is incorporated herein by reference in its entirety, including the compound structures disclosed therein which are specifically incorporated herein by reference.

iix) UBA1

In some embodiments, compositions and methods described herein may include a RAS(ON) inhibitor (e.g., RMC-6236, RMC-6291 and/or RMC-9805) in combination with one or more ubiquitin-activating enzyme inhibitors (e.g., a UBA1 inhibitor). A UBA1 inhibitor may be administered or formulated in combination with a RAS(ON) inhibitor and/or any additional therapeutic agent described herein. UBA1, also known as ubiquitin-activating enzyme 1, is a key enzyme involved in the ubiquitination process, a fundamental cellular mechanism for protein degradation and regulation. Ubiquitination involves the covalent attachment of ubiquitin molecules to target proteins, marking them for degradation by the proteasome or modulating their activity, localization, or interactions within the cell. Several inhibitors have been developed to modulate UBA1 activity, with the aim of disrupting ubiquitination-mediated processes in diseased cells. These inhibitors include but are not limited to adenosine-based inhibitors which typically compete with ATP for binding to the active site of UBA1, thereby preventing the activation of ubiquitin (e.g., PYR-41 and MLN7243); covalent inhibitors which form irreversible bonds with specific amino acid residues in the active site of UBA1, leading to inhibition of its activity (e.g., TAK-243 (formerly known as MLN4924)); allosteric inhibitors which bind to sites on UBA1 distinct from the active site, inducing conformational changes that inhibit its catalytic activity (e.g., compound 2i); and fragment-based inhibitors which are designed based on smaller molecular fragments that bind to UBA1. In some embodiments, a UBA1 inhibitor is one or more of PYR-41, MLN7243, and TAK-243. In some embodiments, reference to the term UBA1 inhibitor includes any such UBA1 inhibitor disclosed in any one of the following patent applications: WO 2016069393 A1, WO 2016069392 A1, and JP 2013237627 A2, each of which is incorporated herein by reference in its entirety, including the compound structures disclosed therein which are specifically incorporated herein by reference.

### m) Additional Therapeutic Agents Useful for Combination Therapy

In some embodiments, compositions and methods described herein may include a RAS(ON) inhibitor (e.g., RMC-6236, RMC-6291 and/or RMC-9805) in combination with one or more Farnesyl transferase inhibitors. A farnesyl transferase inhibitor may be administered or formulated in combination with a RAS(ON) inhibitor and/or any additional therapeutic agent described herein. Farnesyl transferase inhibitors (FTIs) are a class of drugs that target the farnesyl transferase enzyme, which plays a role in a process called protein prenylation. Protein prenylation is an important step in the process of activating certain proteins involved in signal transduction, cell growth, and differentiation. In some embodiments, a farnesyl transferase inhibitor is one or more of tipifarnib, lonafarnib, and rilapladib. In some embodiments, reference to the term farnesyl transferase inhibitor includes any such famesyl transferase inhibitor disclosed in any one of the following patent applications: WO 2010057028, WO 2007042465, WO 200136395, WO 200064891, WO 200042849, WO 199938862. WO 199928315, WO 199829390, WO 199426723, CN 107312000, CN 107365310, KR 100375421, KR 100388790. each of which is incorporated herein by reference in its entirety, including the compound structures disclosed therein which are specifically incorporated herein by reference.

In some embodiments, compositions and methods described herein may include a RAS(ON) inhibitor (e.g., RMC-6236, RMC-6291 and/or RMC-9805) in combination with one or more casein kinase inhibitors in combination with a RAS(ON) inhibitor disclosed herein. In some embodiments, a casein inhibitor is, SR-3029, a potent and ATP competitive CK10 and CK18 inhibitor.

In some embodiments, compositions and methods described herein may include one or more FLT3 inhibitors in combination with a RAS(ON) inhibitor disclosed herein. FLT3 (Fms-like tyrosine kinase 3), also known as CD135, is a receptor tyrosine kinase (RTK) that plays a crucial role in regulating hematopoiesis, the process by which blood cells are formed. It is primarily expressed on hematopoietic stem cells (HSCs) and progenitor cells in the bone marrow, where it controls cell proliferation, survival, and differentiation. In some embodiments, a FLT3 inhibitor includes, but are not limited to, midostaurin, gilteritinib, sorafenib, quizartinib, crenolanib, ponatinib and quizartinib.

In some embodiments, compositions and methods described herein may include a RAS(ON) inhibitor (e.g., RMC-6236, RMC-6291 and/or RMC-9805) in combination with one or more one or more TGFβ pathway inhibitors. In some embodiments, compositions and methods described herein may include one or more TGFβ inhibitors. A TGFβ inhibitor may be administered or formulated in combination with a RAS inhibitor therapy and/or any additional therapeutic agent described herein. TGFβ (transforming growth factor beta) is a multifunctional cytokine involved in various cellular processes, including cell growth, differentiation, apoptosis, and immune response. Dysregulation of the TGFβ signaling pathway has been implicated in various diseases, including cancer, fibrosis, and autoimmune disorders. In some embodiments, a TGFβ inhibitor is one or more of galunisertib (LY2157299), and vactosertib (TEW-7197). In some embodiments, a TGFβ inhibitor is one or more of Galunisertib, LY2157299, Fresolimumab, Lerdelimumab, Trabedersen, curcumin, resveratrol and small interfering RNA (SiRNA) to silence TGFβ receptor expression. In some embodiments, reference to the term TGFβ inhibitor includes any such TGFβ inhibitor disclosed in any one of the following patent applications: WO 2023043473, WO 2020104648, WO 2020128850, WO 2016140884, WO 2007018818, WO 2004024159, WO 200226935, WO 2002062753, WO 2002062776, and JP 2012087076, each of which is incorporated herein by reference in its entirety, including the compound structures disclosed therein which are specifically incorporated herein by reference.

In some embodiments, compositions and methods described herein may include a RAS(ON) inhibitor (e.g., RMC-6236, RMC-6291 and/or RMC-9805) in combination with one or more HSP90 inhibitors. A HSP90 inhibitor may be administered or formulated in combination with a RAS(ON) inhibitor and/or any additional therapeutic agent described herein. HSP90, also known as heat shock protein 90, is a molecular chaperone that plays a critical role in regulating the folding, stability, and activity of a large number of client proteins involved in various cellular processes, including cell cycle progression, signal transduction, and apoptosis. In some embodiments, a HSP90 inhibitor is one or more of Geldanamycin and its derivatives (e.g., 17-AAG, 17-DMAG), KOS 953, Radicicol and its derivatives (e.g., PU-H₇₁), SNX-2112, Ganetespib, AT13387, Onalespib, Luminespib, and KW-2478. In some embodiments, reference to the term HSP90 inhibitor includes any such HSP90 inhibitor disclosed in any one of the following patent applications: WO 2021137665, WO 2018200534, WO 2017151425, WO 2015200514, WO 2013053833, WO 2013009657, WO 2013119985, WO 2012138894, WO 2011044394, WO 2009097578, WO 2008115719, CN 105237533, and CN 104030904, each of which is incorporated herein by reference in its entirety, including the compound structures disclosed therein which are specifically incorporated herein by reference.

In some embodiments, compositions and methods described herein may include a RAS(ON) inhibitor (e.g., RMC-6236, RMC-6291 and/or RMC-9805) in combination with one or more Glutathione peroxidase 4 (GPX4) inhibitors. A GPX4 inhibitor may be administered or formulated in combination with a RAS(ON) inhibitor and/or any additional therapeutic agent described herein. GPX4 is an antioxidant enzyme that plays a critical role in protecting cells against oxidative stress-induced cell death. GPX4 catalyzes the reduction of lipid hydroperoxides to their corresponding alcohols and acts as a regulator of ferroptosis, a form of regulated cell death driven by lipid peroxidation. In some embodiments, a GPX4 inhibitor is one or more of RSL3, ML162, DPI7, FINO2, MCB-613, CBS9106, ML210, ODSH, and TLN232. In some embodiments, reference to the term GPX4 inhibitor includes any such GPX4 inhibitor disclosed in any one of the following patent applications: WO 2021132592, US2021244715, and KR 20220115536, each of which is incorporated herein by reference in its entirety, including the compound structures disclosed therein which are specifically incorporated herein by reference.

In some embodiments, compositions and methods described herein may include a RAS(ON) inhibitor (e.g., RMC-6236, RMC-6291 and/or RMC-9805) in combination with one or more NRF2 inhibitors. A NRF2 inhibitor may be administered or formulated in combination with a RAS(ON) inhibitor and/or any additional therapeutic agent described herein. NRF2 is a transcription factor that regulates the expression of genes involved in the cellular antioxidant response, detoxification, and other cytoprotective pathways. It plays a critical role in cellular defense mechanisms against oxidative stress and other forms of cellular damage. In some embodiments, a NRF2 inhibitor is one or more of ML385, Brusatol, CDDO-Im, RTA-408, and trigonelline. In some embodiments, reference to the term NRF2 inhibitor includes any such NRF2 inhibitor disclosed in any one of the following patent applications: WO 2023051088, WO 2021202720, KR 2022013610, and CN 107519168, each of which is incorporated herein by reference in its entirety, including the compound structures disclosed therein which are specifically incorporated herein by reference.

In some embodiments, compositions and methods described herein may include a RAS(ON) inhibitor (e.g., RMC-6236, RMC-6291 and/or RMC-9805) in combination with one or more TEA domain (TEAD) inhibitors. A TEAD inhibitor may be administered or formulated in combination with a RAS(ON) inhibitor and/or any additional therapeutic agent described herein. TEAD is a family of transcription factors that play a key role in regulating gene expression during embryonic development and tissue homeostasis. The four members of the TEAD family (TEAD1-4) are transcriptional co-activators that bind to DNA through their conserved TEA domain and interact with other transcription factors to activate the expression of target genes. In some embodiments, a TEAD inhibitor is one or more of VT-107, a pan-TEAD, VT-104, Verteporfin, CA3, IAG933, K-975, and Statins (see, e.g., Chapeau, Emilie and Schmelzle, Tobias (2023) IAG933, an oral selective YAP1-TAZ/pan-TEAD protein-protein interaction inhibitor (PPIi) with pre-clinical activity in monotherapy and combinations with MAPK inhibitors. Nature cancer). In some embodiments, reference to the term TEAD inhibitor includes any such TEAD inhibitor disclosed in any one of the following patent applications: WO 2023280254, WO 2023031781, WO 2022258040, WO 2020070181 WO 2018185266, and WO 2017064277, each of which is incorporated herein by reference in its entirety, including the compound structures disclosed therein which are specifically incorporated herein by reference.

In some embodiments, compositions and methods described herein may include a RAS(ON) inhibitor (e.g., RMC-6236, RMC-6291 and/or RMC-9805) in combination with one or more NOTCH/Gamma secretase inhibitors. A NOTCH/Gamma secretase inhibitor may be administered or formulated in combination with a RAS(ON) inhibitor and/or any additional therapeutic agent described herein. In some embodiments, a NOTCH/Gamma secretase inhibitor is nirogacestat. In some embodiments, reference to the term NOTCH/Gamma secretase inhibitor includes any such NOTCH/Gamma secretase inhibitor disclosed in any one of the following patent applications: WO 2020208572, WO 2017200969, WO 2014047390, WO 2014047372, WO 2011041336, WO 2010090954, WO 2009008980, WO 2009087130, WO 2007110335, CN 103664904, CN 105560244, and KR 20200077480, each of which is incorporated herein by reference in its entirety, including the compound structures disclosed therein which are specifically incorporated herein by reference.

In some embodiments, compositions and methods described herein may include a RAS(ON) inhibitor (e.g., RMC-6236, RMC-6291 and/or RMC-9805) in combination with one or more Hedgehog inhibitors. A hedgehog inhibitor may be administered or formulated in combination with a RAS(ON) inhibitor and/or any additional therapeutic agent described herein. The hedgehog (Hh) family of proteins are secreted signaling molecules that play a crucial role in embryonic development and tissue homeostasis in adults. The Hh signaling pathway is involved in regulating cell growth, differentiation, and survival. In some embodiments, a hedgehog inhibitor is one or more of Vismodegib (Erivedge), Sonidegib (Odomzo), and Glasdegib (Daurismo). In some embodiments, reference to the term hedgehog inhibitor includes any such hedgehog inhibitor disclosed in any one of the following patent applications: WO 2011063309, and CN 107163028, each of which is incorporated herein by reference in its entirety, including the compound structures disclosed therein which are specifically incorporated herein by reference.

Compositions and methods described herein may include a RAS(ON) inhibitor (e.g., RMC-6236, RMC-6291 and/or RMC-9805) in combination with one or more NFkB pathway inhibitors. In some embodiments, compositions and methods described herein may include one or more NFkB inhibitors. An NFkB inhibitor may be administered or formulated in combination with a RAS inhibitor therapy and/or any additional therapeutic agent described herein. NF-kappa B (NFkB) is a family of transcription factors involved in regulating various cellular processes, including inflammation, immunity, cell survival, and proliferation. Non-limiting examples of NFkB inhibitors include Bortezomib (Velcade), Curcumin, Parthenolide, IKK inhibitors (e.g., IKK-16, BAY 11-7082), Resveratrol, Andrographolide and Proteasome inhibitors (e.g., MG132, lactacystin).

In some embodiments, the additional therapy is the administration of side-effect limiting agents (e.g., agents intended to lessen the occurrence or severity of side effects of treatment. For example, in some embodiments, the RAS(ON) inhibitor can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor).

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the RAS(ON) inhibitor may be used as an adjuvant therapy after surgery. In some embodiments, the RAS(ON) inhibitor may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the RAS(ON) inhibitor can render abnormal cells more sensitive to treatment with radiation for purposes of killing or inhibiting the growth of such cells. Accordingly, this disclosure further relates to a method for sensitizing abnormal cells in a mammal to treatment with radiation which comprises administering to the mammal an amount of a compound of the present disclosure, which amount is effective to sensitize abnormal cells to treatment with radiation. The amount of the compound in this method can be determined according to the means for ascertaining effective amounts of such compounds described herein. In some embodiments, the RAS(ON) inhibitor may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

In some embodiments, a therapeutic agent for combination therapy may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a RAS(ON) inhibitor as disclosed herein include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

An additional therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

An additional therapeutic agent may be an immune modulatory agent. For example, an additional therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, which interacts with a checkpoint protein. In some embodiments, the checkpoint inhibitor is an agent, such as an antibody, which interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/lg fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002. Non-limiting examples of immune modulatory agent includes targets identified in Table 1.

TABLE 1
Exemplary Immune Modulatory Targets

| Target | Biological Function |
|---|---|
| CTLA-4 | Inhibitory Receptor |
| PD-1 | Inhibitory Receptor |
| PD-L1 | Ligand for PD-1 |
| LAG-3 | Inhibitory Receptor |
| B7.1 | Costimulatory Molecule |
| B7-H3 | Inhibitory Ligand |
| B7-H4 | Inhibitory Ligand |
| TIM3 | Inhibitory Receptor |
| VISTA | Inhibitory Receptor |
| CD137 | Costimulatory Molecule |
| OX-40 | Costimulatory Receptor |
| CD40 agonist | Costimulatory Molecule |
| CD40 agonist + FLT3 ligand | Costimulatory Molecule |
| CD27 | Costimulatory Receptor |
| CCR4 | Costimulatory Receptor |
| GITR | Costimulatory Receptor |
| NKG2D | Activating Receptor |
| KIR | Costimulatory Receptor |
| NKG2A | Inhibitory Receptor |
| ENPP1 | Inhibitory Receptor |
| TIGIT | Inhibitory Receptor |
| A2aR | Inhibitory Receptor |
| CD73 | Inhibitory Receptor |
| CD39 | Inhibitory Receptor |
| PVRIG | Inhibitory Receptor |
| IDO | Inhibitory enzyme |
| CSF1R | Inhibitory Receptor |
| LIF | Inhibitory Cytokine |
| CD47 | Inhibitory Receptor |
| SIRPa | Inhibitory Receptor |
| IL-2 | Effector Cytokines |
| IL-15 | Effector Cytokines |
| IL-12 | Effector Cytokines |
| TREM2 | Receptor |
| TGFb | Multifunctional Cytokine |
| CD73/TGFb trap | Multifunctional Cytokine |
| TCR-T cells directed to KRAS^{MUT}, | Cell therapy |
| mesothelin, or PRAME | |
| mRNA cancer vaccines | vaccines |
| BiTEs | Bi-specific T-cell engager |
| Dual EP2/EP4 inhibitor | E-prostanoid receptor |
| Gamma delta T Cells | Cell therapy |
| NK cells | Cell therapy |

CTLA4, cytotoxic T-lymphocyte-associated antigen 4;
LAG3, lymphocyte activation gene 3;
PD-1, programmed cell death protein 1;
PD-L1, PD-1 ligand;
TIM3, T cell membrane protein 3;
VISTA, V-domain immunoglobulin (Ig)-containing suppressor of T-cell activation;
KIR, killer IgG-like receptor,
APC (Antigen Presenting Cells);
TREM2 (Triggering receptor expressed on myeloid cells 2);
TGF-b (Transforming growth factor beta)

An additional therapeutic agent may be an anti-TIGIT antibody, such as MBSA43, BMS-986207, MK-7684, COM802, AB154, MTIG7192A or OMP-313M32 (etigilimab).

An additional therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irinotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18: 233a (1999), and Douillard et al., Lancet 355 (9209): 1041-1047 (2000).

Other non-limiting examples of anti-cancer agents include Gleevec® (Imatinib Mesylate); Kyprolis® (carfilzomib): Velcade® (bortezomib): Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4 (5)-imidazoles; 4-hydroxytamoxifen; trioxifene; keoxifene; LY 117018; onapristone; toremifene (Fareston®); flutamide, nilutamide, bicalutamide, leuprolide. goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelaamine and thiotepa), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), cell surface monoclonal antibodies (e.g., anti-CD38 (HUMAX-CD38), anti-CSI (e.g., elotuzumab, P13K/Akt inhibitors (e.g., perifosine), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), and cFMS inhibitors (e.g., ARRY-382).

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing. In some embodiments, the anti-cancer agent is JAB-3312.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune modulatory therapies, such as an immune checkpoint inhibitor. In some embodiments, a therapeutic agent may be a pan-RTK inhibitor, such as afatinib.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a RAS(ON) inhibitor of the present disclosure is used in combination with a MEK inhibitor and a SOS1 inhibitor. In some embodiments, a RAS(ON) inhibitor of the present disclosure is used in combination with a PD-L1 inhibitor and a SOS1 inhibitor. In some embodiments, a RAS(ON) inhibitor of the present disclosure is used in combination with a PD-L1 inhibitor and a SHP2 inhibitor. In some embodiments, a RAS(ON) inhibitor of the present disclosure is used in combination with a MEK inhibitor and a SHP2 inhibitor. In some embodiments, the cancer is colorectal cancer, and the treatment comprises administration of a Ras inhibitor of the present disclosure in combination with a second or third therapeutic agent.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al.,

Blood 2007, 110 (1): 186-192; Thompson et al., Clin. Cancer Res. 2007, 13 (6): 1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. No. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the RAS(ON) inhibitor is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), EGFR inhibitory agents (e.g., antibodies or antigen binding regions that specifically bind thereto) such as Vectibix® (panitumumab), erlotinib (Tarceva®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXIGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with a RAS(ON) inhibitor as disclosed herein include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with a RAS(ON) inhibitor is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-la, interferon beta-lb, interferon gamma, natural interferon gamma-la, interferon gamma-lb, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with a RAS(ON) inhibitor include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The disclosure provides pharmaceutical compositions including one or more RAS(ON) inhibitors in combination with one or more therapeutic agents disclosed herein, or a pharmaceutically acceptable salt thereof, as active agents, and a pharmaceutically acceptable excipient.

In some embodiments, active agents in a pharmaceutical composition are in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained release formulation; topical application, for example, as a cream, ointment, or a controlled release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Combination therapies described herein, whether expressly stated or not, may be provided or utilized in salt form, e.g., a pharmaceutically acceptable salt form, unless expressly stated to the contrary.

The RAS(ON) inhibitor may have ionizable groups so as to be capable of preparation as pharmaceutically acceptable salts. These salts may be acid addition salts involving inorganic or organic acids or the salts may, in the case of acidic forms of the RAS(ON) inhibitor, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2 optionally substituted hydroxyl ethanesulfonate, lactobionate, lactate, laurate, lauryl sulfate, malate, maleate, malonate, methanesulfonate, 2 naphthalenesulfonate, nicotinate, nitrate, oleate, oxalate, palmitate, pamoate, pectinate, persulfate, 3 phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

For use as treatment of subjects, the RAS(ON) inhibitor, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988 1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present disclosure, or pharmaceutically acceptable salt thereof, by weight or volume. In some embodiments, compounds, or a pharmaceutically acceptable salt thereof, described herein may be present in amounts totaling 1 95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. U.S. Pat. No. 5,624,677.

Systemic administration may also include relatively noninvasive methods such as the use of suppositories, transdermal patches, transmucosal delivery and intranasal administration. Oral administration is also suitable for compounds of the disclosure, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl polylactic acid, cellulose acetate butyrate, polyvinyl chloride, polyvinyl acetate, vinyl pyrrolidone, polyethylene, polymethacrylate, methylmethacrylate, 2 optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate methyl methacrylate, polyvinyl chloride, polyethylene, or halogenated fluorocarbon.

The liquid forms in which the compounds, or a pharmaceutically acceptable salt thereof, and compositions of the present disclosure can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the disclosure, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein. In certain embodiments, RMC-6236 may be administered at a dose between about 10 mg to 500 mg daily. In certain embodiments, RMC-6236 may be administered at a dose between about 200 mg to 400 mg daily. In certain embodiments, RMC-6236 may be administered at a dose of about 300 mg daily. In certain embodiments, RMC-6291 may be administered at a dose between about 50 mg to 800 mg daily. In certain embodiments, RMC-6291 may be administered at a dose between about 200 mg to 600 mg daily. In certain embodiments, RMC-6291 may be administered at a dose of about 400 mg daily (e.g., 200 mg twice daily). In certain embodiments, RMC-9805 may be administered at a dose between about 150 mg to 2000 mg daily. In certain embodiments, RMC-9805 may be administered at a dose between about 1000 mg to 1400 mg daily.

In some embodiments, the pharmaceutical composition may further include an additional compound having antiproliferative (e.g., anti-cancer) activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present disclosure can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

In one aspect, the present disclosure is directed to methods of treating a disease or disorder that is characterized by aberrant RAS activity (e.g., cancer or a RASopathy). In some embodiments the disease or disorder is cancer (e.g., a cancer having one or more RAS mutations that cause aberrant RAS activity). Non-limiting examples of non-cancerous RAS related diseases or disorders are shown in Table 2. In each embodiment, the method generally comprises administering to the subject a therapeutically effective amount of a RAS(ON) inhibitor in combination with one or more therapeutic agents. Suitable RAS(ON) inhibitors and additional therapeutic agents are described above.

TABLE 2
Exemplary RAS related Non-cancerous Indications

| Disease or disorder | | References |
|---|---|---|
| Immune | Autoimmune disease | Journal of Clinical Immunology |
| disease | | vol. 35, pp. 454-458 (2015) |
| | Rheumatoid arthritis | The Open Rheumatoid Journal vol. |
| | | 6, pp. 259-272 (2012) |
| | RAS-related | PNAS vol. 104, pp. 8953-8958 |
| | autoimmune | (2007) |
| | lymphoproliferative | Blood vol. 117, pp. 2887-2890 |
| | disorders | (2011) |
| Infection | Influenza | Cancer Research vol. 61, pp. |
| | | 8188-8193 (2001) |
| | | PloS ONE vol. 6, el6324 (2011) |
| | | Seikagaku: The Journal of the |
| | | Japanese Biochemical Society vol. |
| | | 87, Issue 1 |
| | EBV infection | Oncogene vol. 23, pp. 8619-8628 |
| | | (2004) |
| | HIV infection | Journal of Biological Chemstry |
| | | vol. 275, pp. 16513-16517 (2000) |
| Neurologic | Alzheimer's disease | Biochimica et Biophysica Acta vol. |
| disease | | 1802, pp. 396-405 (2010) |
| | | Neurobiology of Disease vol. 43, |
| | | pp. 38-45 (2011) |
| | Parkinson's disease | Biochimica et Biophysica Acta vol. |
| | | 1802, pp. 396-405 (2010) |
| | ALS | Biochimica et Biophysica Acta vol. |
| | | 1802, pp. 396-405 (2010) |
| RAS/MAPK | Noonan Syndrome | Human Molecular Genetics vol. |
| syndrome | | 15, pp. R220-R226 (2006) |
| | Costello syndrome | Genetics in Medicine vol. 14, pp. |
| | | 285-292 (2012) |
| | CFC syndrome | Human Mutation vol. 29, pp. |
| | | 992-1006 (2008) |
| Other | Cirrhosis/Chronic | Gastroenterologia Japonica vol. 24, |
| diseases or | hepatitis | pp. 270-276 (1989) |
| disorders | Memory impairment | Nature Communications vol. 7, |
| | | 12926 (2016) |

The disclosure also provides a method of treating cancer in a subject in need thereof, wherein the cancer includes a mutation in RAS. In one embodiment, the addition of a RAS(ON) inhibitor synergistically increases the activity one or more additional therapeutic agents. Any method for determining whether two or more therapeutic agents exhibit synergy may be used for determining the synergistic effect of the combination, such as methods described herein.

Several mathematical models have been developed to determine whether two compounds act synergistically, i.e., beyond a mere additive effect. For instance, Loewe Additivity (Loewe (1928) Physiol. 27:47-187), Bliss Independence (Bliss (1939) Ann. Appl. Biol. 26:585-615), Highest Single Agent, ZIP (Yadav et al (2015) Comput Struct Biotech J 13:504-513) and other models (Chou & Talalay (1984) Adv Enzyme Regul 22:27-55. #6382953; and Greco et al. (1995) Pharmacol Rev 47 (2): 331-85. #7568331) are well known models in the pharmaceutical industry and may be used to calculate a “synergy score” that indicates whether synergy was detected and the magnitude of such synergy. Additional models for determining synergy of two compounds can be found in the examples below.

In general, the mathematical models use data obtained from single agent values to determine the predicted additive effect of the combination which is compared to the observed effect for the combination. If the observed effect is greater than the predicted effect, the combination is deemed to be synergistic. For example, the Bliss independence model compares the observed combination response (Yo) with the predicted combination response (Yp), which was obtained based on the assumption that there is no effect from drug-drug interactions. Typically, the combination effect is declared synergistic if Yo is greater than Yp.

In some embodiments, “synergistic effect” as used herein refers to combination of a RAS(ON) inhibitor in combination with one or more additional therapeutic agents, for example, any of the beneficial or desired results including in vitro results as well as clinical results or endpoints as described herein, which is greater than the sum of the effect observed when a RAS(ON) inhibitor or additional therapeutic agent are administered alone.

The RAS(ON) inhibitor and one or more additional therapeutic agents may be administered simultaneously or sequentially. The RAS(ON) inhibitor and the one or more additional therapeutic agents may be administered as a single formulation or in separate formulations. In some embodiments, the RAS(ON) inhibitor is administered for a first period of time; and the one or more additional therapeutic agents is administered for a second period of time, wherein the first period of time and the second period of time do not overlap and the first period of time precedes the second period of time; and the one or more additional therapeutic agents and RAS(ON) inhibitor are administered for a second period of time, wherein the first period of time and the second period of time do not overlap and the first period of time precedes the second period of time.

In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. In some embodiments, the cancer is non-small cell lung cancer. In some embodiments, the cancer is pancreatic cancer.

In some embodiments, the combination therapies may be used for the treatment of a wide variety of cancers such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example: Cardiac, for example: sarcoma (angiosarcoma, fibrosarcoma, rhabdomyosarcoma, liposarcoma), myxoma, rhabdomyoma, fibroma, lipoma and teratoma; Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, lipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver, for example: hepatoma (hepatocellular carcinoma), cholangiocarcinoma, hepatoblastoma, angiosarcoma, hepatocellular adenoma, hemangioma; Biliary tract, for example: gall bladder carcinoma, ampullary carcinoma, cholangiocarcinoma; Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors; Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma); Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic, for example: blood (myeloid leukemia (acute and chronic), acute lymphoblastic myeloproliferative neoplasms), multiple myeloma, myelodysplastic syndrome), Hodgkin's disease, non-Hodgkin's lymphoma (malignant lymphoma); Skin, for example: malignant melanoma, basal cell carcinoma, squamous cell carcinoma, Kaposi's sarcoma, moles dysplastic nevi, lipoma, angioma, dermatofibroma, keloids, psoriasis; and Adrenal glands, for example: neuroblastoma.

In some embodiments, the cancer includes a RAS mutation, such as a RAS mutation described herein. In some embodiments, a mutation is selected from: the following KRAS mutants: G12D, G12V, G12C, G13D, G12R, G12A, G12S, A146T, G13C, K117N, A146V, G12F, L19F, Q22K, V14I, A59T, A146P, G13R, G12L, or G13V, and combinations thereof; the following HRAS mutants: G13R, G12S, G12D, G13V, G13D, G12C, K117N, A59T, G12V, G13C, G13S, A18V, D119N, G13N, A146T, A66T, G12A, A146V, G12N, or G12R, and combinations thereof; and the following NRAS mutants: G12D, G13R, G13D, G12S, G12C, G12V, G12A, G13V, G12R, P185S, G13C, A146T, G60E, A59D, E132K, E49K, T50I, A146V, or A59T, and combinations thereof; or a combination of any of the foregoing. In some embodiments, the cancer includes a KRAS mutation selected from the group consisting of G12C, G12D, G13C, G12V, G13D, G12R, and G12S. In some embodiments, the cancer includes an NRAS mutation at G12C. In some embodiments, the cancer includes a RAS mutation selected from the group consisting of G12C, G13C, G12A, G12D, G13D, G12S, G13S, G12V and G13V. In some embodiments, the cancer includes at least two RAS mutations selected from the group consisting of G12C, G13C, G12A, G12D, G13D, G12S, G13S, G12V and G13V.

In some embodiments, a compound of the present disclosure binds to or inhibits more than one RAS mutant. In some embodiments, a compound may inhibit both KRAS G12D and KRAS G12V. In some embodiments, a compound may bind to or inhibit both KRAS G12V and KRAS G12S. In some embodiments, a compound of the present disclosure binds to or inhibits wild-type RAS in addition to a RAS mutant. In some embodiments, a compound of the present disclosure binds to or inhibits RAsamp in addition to one or more additional RAS mutations (e.g., K-, H- or NRAsamp and KRAS G12D, G12V, G12C, G13D, G12R, G12A, G12S, A146T, G13C, K117N, A146V, G12F, L19F, Q22K, V14I, A59T, A146P, G13R, G12L, or G13V; K-, H- or NRAsamp and HRAS, G13R, G12S, G12D, G13V, G13D, G12C, K117N, A59T, G12V, G13C, G13S, A18V, D119N, G13N, A146T, A66T, G12A, A146V, G12N, or G12R; or K-, H- or NRAsamp and NRAS G12D, G13R, G13D, G12S, G12C, G12V, G12A, G13V, G12R, P185S, G13C, A146T, G60E, A59D, E132K, E49K, T50I, A146V, or A59T).

In some embodiments, the cancer is non-small cell lung cancer, and the RAS mutation includes a KRAS mutation, such as KRAS G12C, KRAS G12V or KRAS G12D. In some embodiments, the cancer is colorectal cancer, and the RAS mutation includes a KRAS mutation, such as KRAS G12C, KRAS G12V or KRAS G12D. In some embodiments, the cancer is pancreatic cancer, and the RAS mutation includes an NRAS mutation, such as NRAS G12D. In some embodiments, the cancer is melanoma.

In some embodiments, a cancer includes a RAS mutation and an STK11LoF, a KEAP1, an EPHA5 or an NF1 mutation. In some embodiments, the cancer is non-small cell lung cancer and includes a KRAS G12C mutation. In some embodiments, the cancer is non-small cell lung cancer and includes a KRAS G12C mutation and an STK11LoF mutation. In some embodiments, the cancer is non-small cell lung cancer and includes a KRAS G12C mutation and an STK11LoF mutation. In some embodiments, a cancer includes a KRAS G13C RAS mutation and an STK11LoF, a KEAP1, an EPHA5 or an NF1 mutation. In some embodiments, the cancer is non-small cell lung cancer and includes a KRAS G12D mutation. In some embodiments, the cancer is non-small cell lung cancer and includes a KRAS G12V mutation. In some embodiments, the cancer is colorectal cancer and includes a KRAS G12C mutation. In some embodiments, the cancer is pancreatic cancer and includes a KRAS G12D mutation. In some embodiments, the cancer is pancreatic cancer and includes a KRAS G12V mutation. In some embodiments, the cancer is endometrial cancer and includes a KRAS G12C mutation. In some embodiments, the cancer is gastric cancer and includes a KRAS G12C mutation.

Methods for detecting a mutation in a KRAS, HRAS or NRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C KRAS, HRAS or NRAS mutations by real-time PCR. In real-time PCR, fluorescent probes specific for the KRAS, HRAS or NRAS G12C mutation are used. When a mutation is present, the probe binds, and fluorescence is detected. In some embodiments, the KRAS, HRAS or NRAS G12C mutation is identified using a direct sequencing method of specific regions (e.g., exon 2 or exon 3) in the KRAS, HRAS or NRAS gene. This technique will identify all possible mutations in the region sequenced.

Methods for detecting a mutation in a KRAS, HRAS or NRAS protein are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing. Other methods include ctDNA measurement (e.g., Cescon et al., Nature Cancer 1:276-290 (2020)), and utilization of a high-sensitivity diagnostic assay (with CE-IVD mark), e.g., as described in Domagala, et al., Pol J Pathol 3:145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

Methods for determining whether a tumor or cancer includes a G12C or other KRAS, HRAS or NRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a (CTC) sample. In some embodiments, the sample is processed to a cell lysate. In some embodiments, the sample is processed to DNA or RNA.

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

In various embodiments, the disclosure provides a method of treating cancer in a subject comprising administering to the subject a RAS(ON) inhibitor in combination with one or more therapeutic agents as described herein, wherein the subject has one or more tumors that are resistant or unresponsive to treatment. In various embodiments, the subject has one or more tumors that are resistant or unresponsive to one or more treatments selected from the group consisting of surgery, radiation, chemotherapy, biologic agents, small molecules, cell-based therapy, hormone therapy, and immunotherapy. In various embodiments, treatment is a standard of care therapy, first-line therapy, second-line therapy, or third-line therapy. In various embodiments, the subject has one or more tumors that have progressed during one or more treatments, wherein the treatments are standard of care therapy, first-line therapy, second-line therapy, or third-line therapy.

First-line therapy is defined as a treatment that is administered to a subject suffering from cancer who has not received any prior treatment. Second-line therapy is defined as treatment that is administered to a subject suffering from cancer who has received prior first-line therapy but experienced disease progression during first-line treatment. Third-line therapy is defined as treatment that is administered to a subject suffering from cancer who has received prior first and second-line treatment but has experienced disease progression during second-line treatment. Each particular type of cancer has a first-line, second-line, and third-line therapy. The first-, second-, and third-line therapies for types of cancer are known in the art. In addition, FDA approved drug labels will indicate if a particular drug is approved as a first-, second-, or third-line therapy.

Several criteria and definitions published in the literature can be used to determine the effect of one or more treatments on tumors in a subject suffering from cancer. Based on these criteria, tumors are defined as “responsive,” “stable,” or “progressive” when they improve, remain the same, or worsen during treatment, respectively.

Examples of the commonly used criteria published in the literature include Response Evaluation Criteria in Solid Tumors (RECIST), Modified Response Evaluation Criteria in Solid Tumors (mRECIST), PET Response Criteria in Solid Tumors (PERCIST), Choi Criteria, Lugano Response Criteria, European Association for the Study of the Liver (EASL) Criteria, Response Evaluation Criteria in the Cancer of the Liver (RECICL), and WHO Criteria in Tumor Response.

In various embodiments, the disclosure provides a method of treating cancer in a subject comprising administering to the subject a combination therapy of the present disclosure, wherein the subject cannot tolerate standard of care therapy, first-line therapy, second-line therapy, or third-line therapy. In various embodiments, the disclosure provides a method of treating cancer in a subject comprising administering to the subject a combination therapy as described herein, wherein the subject has experienced tumor recurrence after surgical resection of the primary tumor. In various embodiments, the disclosure provides a method of treating cancer in a subject comprising administering to the subject a combination therapy as described herein, wherein the subject has a tumor that cannot be surgically removed. In various embodiments, the disclosure provides a method of treating cancer in a subject comprising administering to the subject a combination therapy as described herein, wherein the subject has no treatment options available.

Several therapies used in the treatment of cancer (e.g., chemotherapies) are cytotoxic and are associated with significant side-effects and toxicities that are associated with poor outcomes and poor response to treatment. Prior to administering such treatments, clinicians rely on several assessment tools to help determine the risk of a subject suffering from cancer experiencing treatment related toxicities and adverse events. Based on the results of these assessments, a subject suffering from cancer is considered intolerant to therapy if they are determined to be at increased risk of experiencing therapy-related toxicities and adverse events resulting in poor outcomes. Examples of commonly used assessment tools used in the determination of therapy intolerance include Karnofsky Performance Status (KPS), Eastern Cooperative Oncology Group Performance Status (ECOG PS), Timed Get Up and Go (TUG), Short Physical Performance Battery (SPPB), Comprehensive Geriatric Assessment (CGA), Cancer Aging Research Group (CARG) Score, and Chemotherapy Risk Assessment Scale for High-Age Patients (CRASH).

In some embodiments, the subject's cancer progression is reduced or prevented. Disease progression of a cancer (e.g., a cancer described herein) can be evaluated by any one or more of several established methods. A person of skill in the art can monitor a subject by direct observation in order to evaluate how the symptoms exhibited by the subject have changed (e.g., a decrease or absence of symptoms) in response to a treatment (e.g., a method of treatment disclosed herein). A subject may also be examined by MRI, CT scan, or PET analysis in order to determine if a tumor has metastasized or if the size of a tumor has changed (e.g., decreased in response to a treatment (e.g., a method of treatment described herein)). Optionally, cells can be extracted from the subject through a biopsy or procedure, or tumor DNA can be isolated from the blood of a subject, and a quantitative biochemical analysis can be conducted in order to assess the relative cancer burden and determine the presence or emergence of specific mutations possibly involved in resistance. Based on the results of these analyses, a person of skill in the art may prescribe higher/lower dosages or more/less frequent dosing of a treatment in subsequent rounds of treatment.

In various embodiments, the disclosure provides a method of treating cancer in a subject comprising administering to the subject a combination therapy as disclosed herein. In various embodiments, the administering reduces tumor size or inhibits tumor growth. In various embodiments, the administering induces tumor cell death, apoptosis, or necrosis.

It is contemplated that the methods herein reduce tumor size or tumor burden in the subject or reduce metastasis in the subject. In various embodiments, the methods reduce the tumor size by 10%, 20%, 30% or more. In various embodiments, the methods reduce tumor size by about or at least about 10%, 15%, 20%, 25%, 30%, 35%, 40%, 45%, 50%, 55%, 60%, 65%, 70%, 75%, 80%, 85%, 90%, or 95% or about 100%, or including all values and ranges that lie in between these values.

In one aspect, the present disclosure provides methods for treating a RAS related disorder in a subject where the RAS related disorder pathology is mediated, in part, through increased signaling in the RAS/MAPK pathway. In various embodiments, the method generally comprises administering to the subject a therapeutically effective amount of a combination therapy as disclosed herein. In some embodiments, the RAS related disorder is a RASopathy. A RASopathy is a group of genetic disorders that are caused by mutations in genes involved in the RAS/MAPK signaling pathway. RASopathies are characterized by a range of clinical features and can affect multiple organ systems, including the cardiovascular, musculoskeletal, neurological, and dermatological systems.

In some embodiments, the methods include treating a RASopathy selected from Noonan syndrome, Costello syndrome, cardiofaciocutaneous syndrome, neurofibromatosis type 1, and Legius syndrome. While each RASopathy has unique features, they all share certain similarities, such as facial dysmorphisms, cardiac abnormalities, developmental delays, and an increased risk of certain cancers.

RASopathies are typically diagnosed through a combination of clinical evaluation, genetic testing, and imaging studies. Treatment and management of RASopathies depend on the specific type and severity of the disorder, but may include medication, surgery, and supportive therapies such as physical and occupational therapy.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a RAS(ON) inhibitor and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a RAS(ON) inhibitor and any of the therapies described herein are administered a few minutes apart, or a few hours apart, or a few days apart.

In some embodiments of any of the methods described herein, the first therapy (e.g., a RAS(ON) inhibitor) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The disclosure also features kits including (a) a pharmaceutical composition including an agent (e.g., a RAS(ON) inhibitor) described herein, and (b) a package insert with instructions to perform any of the methods described herein. In some embodiments, the kit includes (a) a pharmaceutical composition including an agent (e.g., a RAS(ON) inhibitor) described herein, (b) one or more additional therapies (e.g., non-drug treatment or therapeutic agent), and (c) a package insert with instructions to perform any of the methods described herein.

As one aspect of the present disclosure contemplates the treatment of the disease or symptoms associated therewith with a combination of pharmaceutically active compounds that may be administered separately, the disclosure further relates to combining separate pharmaceutical compositions in kit form. The kit may comprise two separate pharmaceutical compositions: a compound of the present disclosure, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### EMBODIMENTS

Embodiment 1: A method of treating cancer in a subject in need thereof, the method comprising administering to the subject a therapeutically effective combination of a RAS(ON) inhibitor disclosed herein and an EGFR inhibitor.
Embodiment 2: The method of embodiment 1, wherein the RAS(ON) inhibitor is a RAS^{MULTI} (ON) inhibitor. Embodiment 3: The method of embodiment 1 or 2, wherein the RAS(ON) inhibitor is compound RMC-6236 or RMC-7977.
Embodiment 4: The method of any one of embodiments 1-3, wherein the EGFR inhibitor is erlotinib, gefitinib, Osimertinib, dacomitinib, afatinib, or an anti-EGFR antibody.
Embodiment 5: The method of any one of embodiments 1-4, wherein the EGFR inhibitor is erlotinib, gefitinib, Osimertinib, dacomitinib, or afatinib.
Embodiment 6: The method of any one of embodiments 1-4, wherein the EGFR inhibitor is an anti-EGFR antibody comprising panitumuab or cetuximab.
Embodiment 7: The method of any one of embodiments 1˜4 or 6, wherein the EGFR inhibitor is cetuximab.
Embodiment 8: The method of any one of embodiments 1-7, wherein the cancer comprises a RAS mutation.
Embodiment 9: The method of embodiment 8, wherein the RAS mutation is a KRAS mutation.
Embodiment 10: The method of embodiment, wherein the RAS mutation is a KRAS^{G12X} mutation.
Embodiment 11: The method of embodiment 10, wherein the KRAS^{G12X} mutation is a KRAS^{G12C} or a KRAS^{G12D} mutation.
Embodiment 12: The method of any one of embodiments 8-11, wherein the therapeutically effective combination further comprises a RAS(ON) mutant-selective inhibitor (e.g., a RAS(ON) G12C-selective inhibitor or a RAS(ON) G12D-selective inhibitor).
Embodiment 13. The method of any one of embodiments 1-12, wherein the cancer is colorectal cancer or pancreatic ductal adenocarcinoma.
Embodiment 14: A method of treating cancer in a subject in need thereof, the method comprises administering to the subject a therapeutically effective combination of a RAS(ON) inhibitor disclosed herein and an EGFR inhibitor.
Embodiment 15: The method of embodiment 14, wherein the RAS(ON) inhibitor is a RAS(ON) G12C-selective inhibitor.
Embodiment 16: The method of embodiment 14 or 15, wherein the RAS(ON) inhibitor is RMC-6291 or RMC-4998.
Embodiment 17: The method of any one of embodiments 14-16, wherein the EGFR inhibitor is erlotinib, gefitinib, Osimertinib, dacomitinib, afatinib, or an anti-EGFR antibody.
Embodiment 18: The method of any one of embodiments 14-17, wherein the EGFR inhibitor is erlotinib, gefitinib, Osimertinib, dacomitinib, or afatinib.
Embodiment 19: The method of any one of embodiments 14-17, wherein the EGFR inhibitor is an anti-EGFR antibody comprising panitumuab or cetuximab.
Embodiment 20: The method of any one of embodiments 14-17 or 19, wherein the EGFR inhibitor is cetuximab.
Embodiment 21: The method of any one of embodiments 14-20, wherein the cancer comprises a RAS mutation.
Embodiment 22: The method of embodiment 21, wherein the RAS mutation is a KRAS mutation.
Embodiment 23: The method of embodiment 22, wherein the KRAS mutation is a KRAS^{G12C} mutation.
Embodiment 24. The method of any one of embodiments 14-23, wherein the cancer is colorectal cancer.
Embodiment 25: A method of treating cancer in a subject in need thereof, the method comprising administering to the subject a therapeutically effective combination of a RAS(ON) inhibitor disclosed herein, a chemotherapeutic agent, and optionally an anti-VEGF.
Embodiment 26: The method of embodiment 1, wherein the RAS(ON) inhibitor is a RAS(ON) multi-selective inhibitor and/or a RAS(ON) G12C-selective inhibitor.
Embodiment 27: The method of embodiment 25 or 26, wherein the RAS(ON) multi-selective inhibitor is compound RMC-6236 or RMC-7977.
Embodiment 29: The method of embodiment 25 or 26, wherein the RAS(ON) G12C-selective inhibitor is RMC-6291 or RMC-4998.
Embodiment 28: The method of any one of embodiments 25-29, wherein the chemotherapy is gemcitabine, paclitaxel, cisplatin, 5-flurouracil, nab-paclitaxel, leucovorin calcium (folinic acid), fluorouracil, irinotecan hydrochloride, oxaliplatin, carboplatin, pemetrexed or a combination thereof.
Embodiment 29: The method of any one of embodiments 28, wherein the chemotherapy is gemcitabine and nab-paclitaxel (abraxane).
Embodiment 30: The method of any one of embodiments 28, wherein the chemotherapy is leucovorin calcium (folinic acid), fluorouracil, irinotecan, and oxaliplatin.
Embodiment 31: The method of any one of embodiments 25-30, wherein the cancer comprises a RAS mutation.
Embodiment 32: The method of embodiment 31, wherein the RAS mutation is a KRAS mutation.
Embodiment 33: The method of embodiment 32, wherein the RAS mutation is a KRAS^{G12X} mutation.
Embodiment 34: The method of embodiment 33, wherein the KRAS^{G12X} mutation is a KRAS^{G12C} or a KRAS^{G12D} mutation.
Embodiment 35: The method of any one of embodiments 32-34, wherein the therapeutically effective combination further comprises a RAS(ON) mutant-selective inhibitor (e.g., a RAS(ON) G12D-selective inhibitor).
Embodiment 36. The method of any one of embodiments 28-35, wherein the cancer is colorectal cancer, pancreatic ductal adenocarcinoma or non-small cell lung cancer.
Embodiment 37: The method of embodiment 25, wherein the RAS(ON) inhibitor is RMC-6236 and the chemotherapeutic agent is gemcitabine and nab-paclitaxel or leucovorin calcium (folinic acid), fluorouracil, irinotecan, and oxaliplatin.
Embodiment 38: the method of embodiment 37, wherein the cancer is pancreatic ductal adenocarcinoma.
Embodiment 39: The method of embodiment 38, wherein the cancer comprises a KRAS mutation (e.g., a KRAS allele amplification).
Embodiment 40: A method of treating cancer in a subject in need thereof, the method comprising administering to the subject a therapeutically effective combination of a RAS(ON) inhibitor disclosed herein and a PD-1/PD-L1 inhibitor.
Embodiment 41: The method of embodiment 40, wherein the RAS(ON) inhibitor is a RAS(ON) multi-selective inhibitor and/or a RAS(ON) G12C-selective inhibitor.
Embodiment 42: The method of embodiment 40 or 41, wherein the RAS(ON) inhibitor is RMC-6236 or RMC-7977.
Embodiment 43: The method of embodiment 40 or 41, wherein the RAS(ON) G12C-selective inhibitor is RMC-6291 or RMC-4998

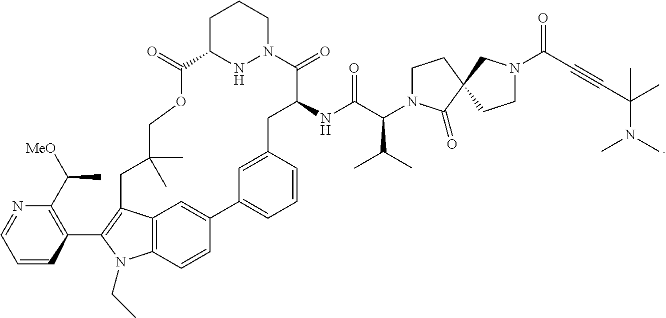

Embodiment 44: The method of any one of embodiments 40-43, wherein the PD-1/PD-L1 inhibitor is a PD-1 inhibitor.
Embodiment 45: The method of any one of embodiments 40-44, wherein the PD-1 inhibitor is nivolumab, pembrolizumab, cemiplimab, tislelizumab, or a biosimilar thereof.
Embodiment 46: The method of any one of embodiments 40-45, wherein the PD-1 inhibitor is pembrolizumab.
Embodiment 47: The method of any one of embodiments 40-46, wherein the PD-1/PD-L1 inhibitor is a PD-L1 inhibitor.
Embodiment 48: The method of embodiment 47, wherein the PD-L1 inhibitor is atezolizumab, avelumab, durvalumab or a biosimilar thereof.
Embodiment 49: The method of any one of embodiments 40-48, wherein the cancer comprises a RAS mutation.
Embodiment 50: The method of embodiment 49, wherein the RAS mutation is a KRAS mutation.
Embodiment 51: The method of embodiment 50, wherein the RAS mutation is a KRAS^{G12X} mutation.
Embodiment 52: The method of embodiment 51, wherein the KRAS^{G12X} mutation is a KRAS^{G12C} or a KRAS^{G12D} mutation.
Embodiment 53: The method of any one of embodiments 45-52, wherein the therapeutically effective combination further comprises a RAS(ON) mutant-selective inhibitor (e.g., a RAS(ON) G12D-selective inhibitor).
Embodiment 54. The method of any one of embodiments 40-53, wherein the cancer is colorectal cancer, pancreatic ductal adenocarcinoma or non-small cell lung cancer.
Embodiment 55: A method of treating cancer in a subject in need thereof, the method comprising administering to the subject a therapeutically effective combination of a first RAS(ON) inhibitor and a second RAS(ON) inhibitor where the first and second RAS(ON) inhibitors are distinct compounds.
Embodiment 56: The method of embodiment 55, wherein the first RAS(ON) inhibitor is a RAS(ON) multi-selective inhibitor.
Embodiment 57: The method of embodiments 55 or 56, wherein the second RAS(ON) inhibitor is a RAS(ON) G12C-selective inhibitor.
Embodiment 58: The method of and one of embodiments 55-57, wherein the RAS(ON) inhibitor is RMC-6236.
Embodiment 59: The method of any one of embodiments 55-58, wherein the RAS^{G12C}(ON) inhibitor is RMC-6291.
Embodiment 60: The method of any one of embodiments 55-59, wherein the cancer comprises a RAS mutation.
Embodiment 61: The method of embodiment 60, wherein the RAS mutation is a KRAS mutation.
Embodiment 62: The method of embodiment 61, wherein the RAS mutation is a KRAS^{G12X} mutation.
Embodiment 63: The method of embodiment 62, wherein the KRAS^{G12X} mutation is a KRAS^{G12C} or a KRAS^{G12D} mutation.
Embodiment 64. The method of any one of embodiments 55-63, wherein the cancer is colorectal cancer, pancreatic ductal adenocarcinoma or non-small cell lung cancer.
Embodiment 65: The method of any one of embodiments 55-64, wherein the cancer is resistant or has developed resistance to the RAS(ON) G12C-selective inhibitor.
Embodiment 66: The method of embodiment 65, wherein the combination of the RAS(ON) G12C-selective inhibitor and the RAS(ON) multi-selective inhibitor is effective in treating the resistant cancer.
Embodiment 67: A method of treating cancer in a subject in need thereof, the method comprising administering to the subject a therapeutically effective combination of a RAS(ON) inhibitor disclosed herein and mTOR inhibitor.
Embodiment 68: The method of embodiment 67, wherein the RAS(ON) inhibitor is RMC-6236 or RMC-7977.
Embodiment 69: The method of embodiment 67 or 68, wherein the mTOR inhibitor is RMC-5552.
Embodiment 70: The method of any one of embodiments 67-69, wherein the cancer comprises a RAS mutation.
Embodiment 71: The method of embodiment 70, wherein the RAS mutation is a KRAS mutation.
Embodiment 72: The method of embodiment 71, wherein the RAS mutation is a KRAS^{G12R} mutation.
Embodiment 73: The method of any one of embodiments 67-72, wherein the cancer is colorectal cancer, pancreatic ductal adenocarcinoma or non-small cell lung cancer.
Embodiment 74: A method of treating cancer in a subject in need thereof, the method comprising administering to the subject a therapeutically effective combination of a RAS(ON) inhibitor disclosed herein and PI3K inhibitor.
Embodiment 75: The method of embodiment 74, wherein the RAS(ON) inhibitor is RMC-6236 or RMC-7977.
Embodiment 76: The method of any one of embodiments 74-75, wherein the cancer comprises a RAS mutation.
Embodiment 77: The method of embodiment 76, wherein the RAS mutation is a KRAS mutation.
Embodiment 78: The method of embodiment 77, wherein the RAS mutation is a KRAS^{G12C} mutation.
Embodiment 79: The method of embodiment 78, wherein the cancer is resistant to a G12C mutant-selective inhibitor.
Embodiment 80: The method of any one of embodiments 74-79, wherein the cancer is colorectal cancer, pancreatic ductal adenocarcinoma or non-small cell lung cancer.
Embodiment 81: A method of treating cancer in a subject in need thereof, the method comprising administering to the subject a therapeutically effective combination of a RAS(ON) inhibitor disclosed herein and a YAP/TAZ-TEAD pathway inhibitor.
Embodiment 82: The method of embodiment 74, wherein the RAS(ON) inhibitor is RMC-6236 or RMC-7977.
Embodiment 83: The method of embodiment 81 or 82, wherein the YAP/TAZ-TEAD pathway inhibitor is a pan-TEAD inhibitor.
Embodiment 84: The method of any one of embodiments 81-83, wherein the TEAD inhibitor is one or more of VT-107, VT-104, Verteporfin, CA3, a Statin, K-975, or IAG933.
Embodiment 85: The method of any one of embodiments 81-84, wherein the cancer comprises a RAS mutation.
Embodiment 86: The method of embodiment 85, wherein the RAS mutation is a KRAS mutation.
Embodiment 87: The method of any one of embodiments 81-86, wherein the cancer is colorectal cancer, pancreatic ductal adenocarcinoma or non-small cell lung cancer.
Embodiment 88: A method of treating cancer in a subject in need thereof, the method comprising administering to the subject a therapeutically effective combination of a RAS(ON) inhibitor disclosed herein and a CDK4/6 inhibitor.
Embodiment 89: The method of embodiment 88, wherein the RAS(ON) inhibitor is RMC-6236 or RMC-7977.
Embodiment 90: The method of embodiment 88 or 89, wherein the CDK4/6 inhibitor is palbociclib.
Embodiment 91: The method of any one of embodiments 88-90, wherein the method further comprises administering a CD40 agonist and/or a PD-1/PD-L1 inhibitor.
Embodiment 92: The method of embodiment 91, wherein the CD40 agonist is one or more of CP-870,893, APX005M, selicrelumab (also known as CP-870,893), ADC-1013, and CDX-1140.
Embodiment 93: The method of any one of embodiments 88-92, wherein the cancer comprises a RAS mutation.
Embodiment 94: The method of embodiment 93, wherein the RAS mutation is a KRAS mutation.
Embodiment 95: The method of any one of embodiments 88-94, wherein the cancer is colorectal cancer, pancreatic ductal adenocarcinoma or non-small cell lung cancer.
Embodiment 96: A method of treating cancer in a subject in need thereof, the method comprising administering to the subject a therapeutically effective amount of a combination, comprising i) a RAS(ON) inhibitor; and ii) one or more additional therapeutic agents.
Embodiment 97: A method of treating a RAS related disease or disorder in a subject in need thereof, the method comprising administering to the subject a therapeutically effective amount of a combination, comprising i) a RAS(ON) inhibitor; and ii) one or more additional therapeutic agents.
Embodiment 98: A method of inhibiting RAS activity and activity of one or more target proteins, in a cell, the method comprising administering to the cell an effective amount of a combination, comprising i) a RAS(ON) inhibitor; and ii) one or more additional therapeutic agents, wherein the one more additional therapeutic agents modulate the activity of the one or more target proteins.
Embodiment 99: The method of any one of embodiments 96 to 98, wherein the RAS(ON) inhibitor is selected from a RAS(ON) inhibitor described in section I (A).
Embodiment 100: The method of any one of embodiments 96 to 98, wherein the one or more additional therapeutic agents is a RAS/MAPK pathway inhibitor, a Kinase inhibitor, a Receptor Tyrosine Kinase inhibitor, a PI3K/mTOR pathway inhibitor, a DNA Damage Response inhibitor, a Cell Cycle inhibitor, an Anti-apoptotic protein inhibitor, an Autophagy inhibitor, a Macropinocytosis inhibitor, a Wnt/Beta-catenin pathway inhibitor, a JAK/STAT pathway inhibitor, an Epigenetic modulator, an immunotherapy, a farnesyl transferase inhibitor, a TGFbeta inhibitor, a HSP90 inhibitor, a GPX4 inhibitor, a NRF2 inhibitor, a TEAD inhibitor, a NOTCH inhibitor, a gamma secretase inhibitor, a hedgehog inhibitor, a chemotherapeutic, a proteasome inhibitor, or any combination thereof.
Embodiment 101: The method of embodiment 100, wherein the RAS/MAPK pathway inhibitor is a RAS (OFF) inhibitor, a SOS1 inhibitor, a SHP2 inhibitor, a MEK inhibitor, a RAF inhibitor, a ERK inhibitor, a MAPK inhibitor, or any combination thereof.
Embodiment 102: The method of embodiment 101, wherein the RAS (OFF) inhibitor is selected from a RAS (OFF) inhibitor described in section (b) (i).
Embodiment 103: The method of embodiment 102, wherein the SOS1 inhibitor is a SOS1 inhibitor described in section I (b) (ii) (e.g., RMC-5845, RMC-4948, RMC-0331, BI-1701963, BI-3406, SDR5, MRTX0902, BAY-293, any combination thereof).
Embodiment 104: The method of embodiment 101, wherein the SHP inhibitor is a SHP inhibitor described in section I (b) (iii) (e.g., SHP099, TNO155, RMC-4550, RMC-4630, JAB-3068, JAB-3312, RLY-1971, ERAS-601, SH3809, PF-07284892, BBP-398, or any combination thereof).
Embodiment 105: The method of embodiment 101, wherein the MEK inhibitor is a MEK inhibitor described in section I (b) (iv) (e.g., pimasertib, selumetinib, cobimetinib, trametinib, binimetinib, or any combination thereof).
Embodiment 106: The method of embodiment 101, wherein the RAF inhibitor is a RAF inhibitor described in section I (b) (v) (e.g., VS-6766, IK-595, vemurafenib, dabrafenib, and encorafenib, or any combination thereof).
Embodiment 107: The method of embodiment 101, wherein the ERK inhibitor is an ERK inhibitor described in section (b) (vi) (e.g., ASTX-029, 1-75, or a combination thereof).
Embodiment 108: The method of embodiment 101, wherein the MAPK inhibitor is a MAPK inhibitor described in section I (b) (vii) (e.g., Tilpisertib (GS-4875), neflamapidmod (VX-745), or a combination thereof).
Embodiment 109: The method of embodiment 100, wherein the kinase inhibitor is a PKA inhibitor, a FAK inhibitor, a ROCK inhibitor, a MSK1 inhibitor, a RSK inhibitor, an ALK inhibitor, or any combination thereof.
Embodiment 110: The method of embodiment 109, wherein the PKA inhibitor is a PKA inhibitor described in section I (c) (i) (e.g., H89).
Embodiment 111: The method of embodiment 110, wherein the FAK inhibitor is a FAK inhibitor described in section I (c) (ii) (e.g., BI853520, defactinib, GSK2256098, PF-00562271, VS-4718, or any combination thereof).
Embodiment 112: The method of embodiment 109, wherein the ROCK inhibitor is a ROCK inhibitor described in section I (b) (iii) (e.g., GSK269962A).
Embodiment 113: The method of embodiment 109, wherein the MSK1 inhibitor is a MSK1 inhibitor described in section I (c) (iv) (e.g., SB-747651A, SB 747651A, Ro 320432, CGP 57380, GSK2830371, SR1664, LY-3214996, PFI-4, MSC-2363318A, AS601245, or any combination thereof).
Embodiment 114: The method of embodiment 109, wherein the RSK inhibitor is a RSK inhibitor described in section I (c) (v) (e.g., BI-D1870, LJH685, SL0101-1, FMK, BRD7389, BIX 02565, LJI308, LJI308-S, LJI308-1, LJH685-S, or any combination thereof).
Embodiment 115: The method of embodiment 115, wherein the ALK inhibitor is an ALK inhibitor described in section I (c) (vi) (e.g., Crizotinib, Ceritinib, Alectinib, Brigatinib, Lorlatinib, Ensartinib (X-396), TAE684, ASP3026, TPX-0131, LDK378 (Ceritinib analog), CEP-37440; 4SC-203, TL-398, PLB1003, TSR-011, CT-707, TPX-0005, AP26113, or any combination thereof).
Embodiment 116: The method of embodiment 100, wherein the Receptor Tyrosine Kinase inhibitor is an EGFR inhibitor, a HER2 inhibitor, a MET inhibitor, an AXL inhibitor, an IGFR inhibitor, a RET inhibitor, a ROS1 inhibitor, a PDGFR inhibitor, a FGFR inhibitor, a VEGF inhibitor, or any combination thereof.
Embodiment 117: The method of embodiment 116, wherein the EGFR inhibitor is an EGFR inhibitor described in section I (d) (i) (e.g., osimertinib, cetuximab, gefitinib (Iressa), erlotinib (Tarceva), lazertinib, afatinib (Gilotrif), or any combination thereof).
Embodiment 118: The method of embodiment 116, wherein the HER2 inhibitor is a HER inhibitor described in section I (d) (ii) (e.g., tucatinib).
Embodiment 119: The method of embodiment 116, wherein the MET inhibitor is a MET inhibitor described in section I (d) (iii) (e.g., Crizotinib (Xalkori), Cabozantinib (Cometriq, Cabometyx), Capmatinib (Tabrecta), Tepotinib (Tepmetko), Savolitinib (Volitinib), Onartuzumab (MetMab), Foretinib (GSK1363089), MGCD-265 (Amuvatinib), SU11274, SU5416, or any combination thereof).
Embodiment 120: The method of embodiment 116, wherein the AXL inhibitor is an ALK inhibitor described in section I (d) (iv) (e.g., bemcentib, BGB324, R428, SGI-7079, TP-0903, BMS-777607, UNC2025, TP-0903, or any combination thereof).
Embodiment 121: The method of embodiment 116, wherein the IGFR inhibitor is an IGFR inhibitor described in section I (d) (v) (e.g., linsitinib, AXL1717, OSI-906 (Linsitinib), BMS-754807, BI 836845, AZ12253801, PQIP (Pyrrolo[1,2-a]quinoxaline), NVP-AEW541, or any combination thereof).
Embodiment 122: The method of embodiment 116, wherein the RET inhibitor is a RET inhibitor described in section I (d) (vi) (e.g., pralsetinib, selpercatinib (LOXO-292), BLU-667, RXDX-105, TPX-0046, GSK3179106, molidustat (BAY 85-3934), RPI-1 (Retrophin), or any combination thereof).
Embodiment 123: The method of embodiment 116, wherein the ROS1 inhibitor is a ROS1 inhibitor described in section I (d) (vii) (e.g., taletrectinib, DS-6051b, TPX-0131, GZD824, PF-06463922, or any combination thereof).
Embodiment 124: The method of embodiment 116, wherein the PDGFR inhibitor is a PDGFR inhibitor described in section I (d) (viii) (e.g., CP-673451, imatinib, nintedanib (ofev), sunitinib (sutent), pazopanib (votrient), regorafenib (stivarga), dasatinib (sprycel), or any combination thereof).
Embodiment 125: The method of embodiment 116, wherein the FGF is an FGF inhibitor described in section I (d) (ix) (e.g., futibatinib (TAK-659), erdafitinib (balversa), infigratinib (Truseltiq), Debio 1347, rogaratinib (BAY 1163877), or any combination thereof).
Embodiment 126: The method of embodiment 116, wherein the VEGF inhibitor is a VEGF inhibitor described in section I (d) (x) (e.g., bevacizumab, aflibercept, ramucirumab, sorafenib, sunitinib, pazopanib, or any combination thereof.
Embodiment 127: The method of embodiment 100, wherein the PI3K/mTOR pathway inhibitor is a PI3K inhibitor, an AKT inhibitor, an mTOR inhibitor, an MNK inhibitor, an eIF4 inhibitor, or any combination thereof.
Embodiment 128: The method of embodiment 127, wherein the PI3K inhibitor is a PI3K inhibitor described in section I (e) (i) (e.g., alpelisib, copanlisib, or a combination thereof).
Embodiment 129: The method of embodiment 128, wherein the AKT inhibitor is an AKT inhibitor described in section I (e) (ii) (e.g., ipatasertib, GSK-2141795, Akt-1-1, Akt-1-1,2, a 1-H-imidazo[4,5-c]pyridinyl derivative, indole-3-carbinol or a derivative thereof, perifosine, a phosphatidylinositol ether lipid analog, triciribine, or any combination thereof).
Embodiment 130: The method of embodiment 127, wherein the mTOR inhibitor is an mTOR inhibitor described in section I (e) (iii) (e.g., RMC-5552, PI-103, PP242, PP30, Torin 1, an FKBP12 enhancer, a 4H-1-benzopyran-4-one derivative, rapamycin (sirolimus), a rapalog, temsirolimus, everolimus, ridaforolimus, AP23464, AP23841, 40-(2-hydroxyethyl) rapamycin, 40-[3-hydroxy (hydroxymethyl)methylpropanoate]-rapamycin (CC1779), 40-epi-(tetrazolyt)-rapamycin (ABT578), 32-deoxorapamycin, 16-pentynyloxy-32 (S)-dihydrorapanycin, a phosphorus-containing rapamycin derivative, or any combination thereof).
Embodiment 131: The method of embodiment 127, wherein the MNK inhibitor is an MNK inhibitor described in section I (e) (iv) (e.g., tomivosertib (eFT508), CGP57380, and SEL201, or any combination thereof).
Embodiment 132: The method of embodiment 127, wherein the eIF4 inhibitor is an eIF4A inhibitor or an eIF4G inhibitor.
Embodiment 133: The method of embodiment 127, wherein the eIF4A inhibitor is an eIF4A inhibitor described in section I (e) (v) (e.g., zotatifin (eFT226), silvestrol, pateamine A, a rocaglate, or any combination thereof).
The method of embodiment 132, wherein the eIF4G inhibitor is pateamine A, hippuristanol, or any combination thereof.
Embodiment 134: The method of embodiment 100, wherein the DNA damage response inhibitor is a Wee1 inhibitor, a CHK inhibitor, an ATM inhibitor, an ATR inhibitor, a PARP inhibitor, a DNA-PK inhibitor, or any combination thereof.
Embodiment 135: The method of embodiment 134, wherein the Wee1 inhibitor is a Wee1 inhibitor described in section I (f) (i) (e.g., adavosertib, AZD1775, ZNL-02-096, MK-1775, or any combination thereof.
Embodiment 136: The method of embodiment 134, wherein the CHK inhibitor is a CHK1 or a CHK2 inhibitor.
Embodiment 137: The method of embodiment 136, wherein the CHK inhibitor is a CHK inhibitor described in section I (f) (ii) (e.g., rabusertib, LY2606368, GDC-0575, MK-8776, or any combination thereof).
Embodiment 138: The method of embodiment 137, wherein the ATM inhibitor is an ATM inhibitor described in section I (f) (iii) (e.g., M4076, AZD0156, KU-60019, VE-821, or any combination thereof.
Embodiment 139: The method of embodiment 134, wherein the ATR inhibitor is an ATR inhibitor described in section I (f) (iv) (e.g., ceralasertib, VX-970, AZD6738, BAY 1895344, or any combination thereof).
Embodiment 140: The method of embodiment 134, wherein the PARP inhibitor is a PARP inhibitor described in section I (f) (v) (e.g., olaparib, rucaparib, niraparib, veliparib (ABT-888), or any combination thereof).
Embodiment 141: The method of embodiment 134, wherein the DNA-PK inhibitor is a DNA-PK inhibitor described in section I (f) (vi) (e.g., NU7441, AZD7648, VX-984, M3814, CC-115, SCR7, or any combination thereof.
Embodiment 142: The method of embodiment 100, wherein the cell cycle inhibitor is a CDK inhibitor, an Aurora kinase inhibitor, a PLK inhibitor, a KSP inhibitor, or any combination thereof.
Embodiment 143: The method of embodiment 142, wherein the CDK inhibitor is a CDK2 inhibitor, a CDK4/6 inhibitor, a CDK7 inhibitor, or a CDK9 inhibitor, or any combination thereof.
Embodiment 144: The method of embodiment 143, wherein the CDK inhibitor is a CDK inhibitor described in section I (g) (i) (e.g., seliciclib, UCN-01, P1446A-05, PD-0332991, dinaciclib, P27-00, AT-7519, RGB286638, SCH727965, AZD4573, or any combination thereof.
Embodiment 145: The method of embodiment 142, wherein the Aurora kinase inhibitor is an Aurora Kinase inhibitor described in section I (g) (ii) (e.g., palbociclib, ribociclib, abemaciclib, alisertib, danusertib, barasertib, MLN8237, or any combination thereof).
Embodiment 146: The method of embodiment 142, wherein the PLK inhibitor is a PLK inhibitor described in section I (g) (iii) (e.g., volasertib, onvansertib, BI 2536, GSK461364, or any combination thereof).
Embodiment 147: The method of embodiment 142, wherein the KSP inhibitor is a KSP inhibitor described in section I (g) (iv) (e.g., SB743921, monastrol, S-Trityl-L-cysteine (STLC), filanesib (ARRY-520), AMG650, BTB-1, K03861, SJ000291942, or any combination thereof).
Embodiment 148: The method of embodiment 100, wherein the anti-apoptotic inhibitors is a Bcl inhibitor, an XIAP inhibitor, a survivin inhibitor, an Mcl-1 inhibitor, or a FLIP inhibitor, or any combination thereof.
Embodiment 149: The method of embodiment 148, wherein the Bcl inhibitor is a BCL inhibitor described in section I (g) (v) (e.g., ABT-263, Venetoclax (Venclexta), Navitoclax (ABT-263), A-1331852, S63845, AT-101, or any combination thereof).
Embodiment 150: The method of embodiment 148, wherein the Mcl-1 inhibitor is an Mcl-1 inhibitor described in section I (g) (vi) (e.g., AMG-176, MIK665, S63845, or any combination thereof).
Embodiment 151: The method of embodiment 100, wherein the autophagy inhibitor is chloroquine, 3-methyladenine, hydroxychloroquine, spautin-1, SAR405, bafilomycin A1, 5-amino-4-imidazole carboxamide riboside (AICAR), okadaic acid, autophagy-suppressive algal toxins, analogues of CAMP, LY204002, N6-mercaptopurine riboside, vinblastine, a ULK1 inhibitor, a VPS inhibitor or any combination thereof.
Embodiment 152: The method of embodiment 151, wherein the ULK1 inhibitor is a ULK1/2 inhibitor.
Embodiment 153: The method of embodiment 151, wherein the ULK inhibitor is a ULK inhibitor described in section I (h) (i) (a) (e.g., ULK-101, MRT68921, SBI-0206965, MRT67307, MRT68920, MRT68922, MRT199665, LY3009120, Dorsomorphin, or any combination thereof.
Embodiment 154: The method of embodiment 151, wherein the VPS inhibitor is a VPS inhibitor described in section I (h) (i) (b) (e.g., PIK-III, VPS34-IN1, SAR405, Spautin-1, NSC185058, or any combination thereof).
Embodiment 155: The method of embodiment 100, wherein the macropinocytosis inhibitor is a macropinocytosis inhibitor described in section I (h) (i) (c) (e.g., EIPA (ethylisopropylamiloride), Wortmannin, Amiloride, Apilimod, Dyngo-4a, Latrunculin B, or any combination thereof).
Embodiment 156: The method of embodiment 100, wherein the Wnt/Beta-catenin pathway inhibitor is a beta-catenin inhibitor, a PORCN inhibitor, a GSK3 inhibitor, a CLK inhibitor, or any combination thereof. The method of claim 63, wherein the beta-catenin inhibitor is a beta-catenin inhibitor described in section I (j) (i) (e.g., tegavivant, foscenvivant, PRI-724 (also known as ICG-001), C-82, BC2059, or any combination thereof).
Embodiment 157: The method of embodiment 156, wherein the PORCN inhibitor is a PORCN inhibitor described in section I (j) (ii) (e.g., LGK974 (WNT974), ETC-1922159, CGX1321, CWP232291, or any combination thereof).
Embodiment 158: The method of embodiment 156, wherein the GSK3 inhibitor is a GSK3 inhibitor described in section I (j) (iii) (e.g., Tideglusib, laduviglusib, LiCl (Lithium chloride), CHIR99021, SB216763, AZD1080, LY2090314, or any combination thereof).
Embodiment 159: The method of embodiment 156, wherein the CLK inhibitor is a CLK inhibitor described in section I (j) (iv) (e.g., SM08502, SM04690, TG003, KH-CB19, T3.5, CX-4945, or any combination thereof).
Embodiment 160: The method of embodiment 100, wherein the JAK/STAT pathway inhibitor is an inhibitor of JAK1, JAK2, JAK3, STAT3, STAT5, or any combination thereof.
Embodiment 160: The method of embodiment 160, wherein the JAK inhibitor is Ruxolitinib, Fedratinib, Tofacitinib, Baricitinib, or any combination thereof.
Embodiment 161: The method of embodiment 160, wherein the STAT inhibitor is SD-36, Stattic, S31-201, OPB-31121, Napabucasin (BBI608), or any combination thereof.
Embodiment 162: The method of embodiment 100, wherein the epigenetic modulator is a HDAC inhibitor, a BET inhibitor, an EZH2 inhibitor, a Co-REST inhibitor, an EP300 inhibitor, an LSD1 inhibitor, a PRMT5 inhibitor, an MAT2A inhibitor, a DOTL1 inhibitor, or any combination thereof.
Embodiment 163: The method of embodiment 100, wherein the farnesyl transferase inhibitor is tipifarnib, lonafarnib, rilapladib, or any combination thereof.
Embodiment 164: The method of embodiment 100, wherein the TGFbeta inhibitor is galunisertib (LY2157299), vactosertib (TEW-7197), Fresolimumab, Lerdelimumab, Trabedersen, curcumin, resveratrol, or any combination thereof.
Embodiment 165: The method of embodiment 100, wherein the HSP90 inhibitor is geldanamycin or a derivative (e.g., 17-AAG, 17-DMAG), KOS 953, Radicicol or a derivative (e.g., PU-H₇₁), SNX-2112, Ganetespib, AT13387, Onalespib, Luminespib, KW-2478, or any combination thereof.
Embodiment 166: The method of embodiment 100, wherein the GPX4 inhibitor is RSL3, ML162, DPI7, FINO2, MCB-613, CBS9106, ML210, ODSH, TLN232, or any combination thereof.
Embodiment 167: The method of embodiment 100, wherein the NRF2 inhibitor is ML385, Brusatol, CDDO-Im, RTA-408, trigonelline, or any combination thereof.
Embodiment 168: The method of embodiment 100, wherein the TEAD inhibitor is VT-107, a pan-TEAD inhibitor, K-975, IAG933, VT-104, Verteporfin, CA3, a Statin, or any combination thereof.
Embodiment 169: The method of embodiment 100, wherein the notch/gamma secretase inhibitor is nirogacestat.
Embodiment 170: The method of embodiment 100, wherein the hedgehog inhibitor is Vismodegib, Sonidegib, Glasdegib, or any combination thereof.
Embodiment 171: The method of embodiment 100, wherein the chemotherapeutic is FOLFOX, FOLFIRI, 5-FU, Tipircil, trifluridine, TMZ, docetaxel, gemcitabine, abraxane, paclitaxel, cisplatin, carboplatin, etoposide, or any combination thereof.
Embodiment 172: The method of embodiment 100: wherein the immunotherapy is an immune checkpoint inhibitor, a cytokine inhibitor, a cytokine, a vaccine, an antibody therapy, a bispecific antibody, a cellular therapy, or any combination thereof.
Embodiment 173: The method of embodiment 172, wherein the immunotherapy is described in Table 1.
Embodiment 174: The method of any one of embodiments 96 to 98, wherein the one or more additional therapeutic agent comprises an immune checkpoint inhibitor and chemotherapeutic agent.
Embodiment 175: The method of any one of embodiment 96 to 98, wherein the one or more additional therapeutic agent comprises a COX1/2 inhibitor or a COX1 inhibitor.
Embodiment 176: The method of any one of embodiment 96 to 98, wherein the one or more additional therapeutic agent comprises an xCT inhibitor.
Embodiment 177: The method of embodiment 176, wherein the inhibitor xCT inhibitor is a SLCA11 inhibitor.
Embodiment 178: The method of any one of embodiments 96 to 98, wherein the one or more additional therapeutic agent comprises a PPAR agonist.
Embodiment 179: The method of any one of embodiments 96 to 98, wherein the one or more additional therapeutic agent comprises a menin inhibitor.
Embodiment 180: The method of any one of embodiments 96 to 98, wherein the one or more additional therapeutic agent comprises a PTEN stabilizer.
Embodiment 181: The method of any one of embodiments 96 to 98, wherein the one or more additional therapeutic agent comprises a SGK inhibitor.
Embodiment 182: The method of any one of embodiments 96 to 98, wherein the one or more additional therapeutic agent comprises a myc inhibitor.
Embodiment 183: The method of any one of embodiments 96 to 98, wherein the one or more additional therapeutic agent comprises a DLL3 inhibitor.
Embodiment 184: The method of any one of embodiments 96 to 98, wherein the one or more additional therapeutic agent comprises a CCR8 inhibitor.
Embodiment 185: The method of any one of embodiments 96 to 98, wherein the one or more additional therapeutic agent comprises a Sting agonist.
Embodiment 186: The method of any one of embodiments 96 to 98, wherein the one or more additional therapeutic agent comprises a SCD1 inhibitor.
Embodiment 187: The method of any one of embodiments 96 to 98, wherein the one or more additional therapeutic agent comprises a GSPT1 inhibitor.
Embodiment 188: The method of embodiments 182 or 187, wherein the inhibitor is MRT-2359.
Embodiment 189: The method of any one of embodiments 96 to 98, wherein the one or more additional therapeutic agent comprises a NEK7 modulator.
Embodiment 190: The method of any one of embodiments 96 to 98, wherein the one or more additional therapeutic agent comprises a hormone receptor modulator.
Embodiment 191: The method of any one of embodiments 96 to 98, wherein the one or more additional therapeutic agents comprises a CTLA-4 inhibitor.
Embodiment 192: The method of embodiment 191, wherein the CTLA-4 inhibitor is botensilimab.
Embodiment 193: The method of embodiment 192, wherein the combination further comprises an immune check point inhibitor.
Embodiment 194: The method of embodiment 193, wherein the immune checkpoint inhibitor is balstilimab.
Embodiment 195: The method of any one of embodiments 96 to 98, wherein the combination comprises an EGFR inhibitor and pembrolizumab.
Embodiment 196: The method of any one of embodiments 96 to 98, wherein the combination comprises a SHP2 inhibitor, an immune checkpoint inhibitor and CTLA-4 inhibitor.
Embodiment 197: The method of any one of embodiments 96 to 196, wherein the combination further comprises one or more pharmaceutically acceptable excipients.
Embodiment 198: A pharmaceutical composition of embodiment 197.
Embodiment 199: The pharmaceutical composition of embodiment 198 for use in a method of treating a RAS related disease or disorder (e.g., a cancer), the method comprising administering the pharmaceutical composition to the subject in need thereof.
Embodiment 200: The method of any one of embodiments 96 to 199, wherein the RAS(ON) inhibitor(s) and the one or more additional therapeutic agent(s) are administered simultaneously.
Embodiment 201: The method of any one of embodiments 96 to 199, wherein the RAS(ON) inhibitor(s) and the one or more additional therapeutic agent(s) are administered sequentially.
Embodiment 202: The method of any one of embodiments 96 to 199, wherein the RAS(ON) inhibitor(s) and the one or more additional therapeutic agent(s) are formulated in a single composition.
Embodiment 203: The method of any one of embodiments 96 to 199, wherein the RAS(ON) inhibitor(s) and the one or more additional therapeutic agent(s) are formulated in separate compositions.
Embodiment 203: The method of any one of embodiments 96 to 203, wherein the RAS related disease or disorder is cancer, or the cell is a cancer cell.
Embodiment 204: The method of embodiment 203, wherein the cancer is astrocytic, breast, cervical, colorectal, endometrial, esophageal, gastric, head and neck, hepatocellular, laryngeal, lung, oral, ovarian, prostate, and thyroid carcinomas and sarcomas. Other cancers include, for example: Cardiac, for example: sarcoma (angiosarcoma, fibrosarcoma, rhabdomyosarcoma, liposarcoma), myxoma, rhabdomyoma, fibroma, lipoma and teratoma; Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, lipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver, for example: hepatoma (hepatocellular carcinoma), cholangiocarcinoma, hepatoblastoma, angiosarcoma, hepatocellular adenoma, hemangioma; Biliary tract, for example: gall bladder carcinoma, ampullary carcinoma, cholangiocarcinoma; Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors; Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma); Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic, for example: blood (myeloid leukemia (acute and chronic), acute lymphoblastic myeloproliferative neoplasms), multiple myeloma, myelodysplastic syndrome), Hodgkin's disease, non-Hodgkin's lymphoma (malignant lymphoma); Skin, for example: malignant melanoma, basal cell carcinoma, squamous cell carcinoma, Kaposi's sarcoma, moles dysplastic nevi, lipoma, angioma, dermatofibroma, keloids, psoriasis; and Adrenal glands, for example: neuroblastoma.
Embodiment 205: The method of embodiment 204, wherein the cancer is a lung cancer (e.g., NSCLC) or a gastric cancer (e.g., PDAC, or CRC).
Embodiment 206: The method of any one of embodiments 96 to 205, wherein the subject has increased progression-free survival relative the progression-free survival of a subject or subject population receiving a monotherapy with the RAS(ON) inhibitor or the additional therapeutic agent.
Embodiment 207: The method of any one of embodiments 96 to 205, wherein the subject has increased probability of relapse-free survival relative the relapse-free survival of a subject or subject population receiving a monotherapy with the RAS(ON) inhibitor or the additional therapeutic agent.
Embodiment 208: The method of any one of embodiments 96 to 205, wherein the subject has increased anti-tumor activity relative to anti-tumor activity in a subject or subject population receiving a monotherapy with the RAS(ON) inhibitor or the additional therapeutic agent.
Embodiment 209: The method according to any one of embodiments 1 to 208, wherein the one or more additional therapeutic agents synergistically increases the sensitivity of the cancer cells to the RAS(ON) inhibitor.
Embodiment 210: The method according to any one of embodiments 1 to 208, wherein the RAS(ON) inhibitor synergistically increases the sensitivity of the cancer cells to the one or more additional therapeutic agents.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1. Improved Responses and Prolonged Durability with RAS(ON) Inhibitor Doublet

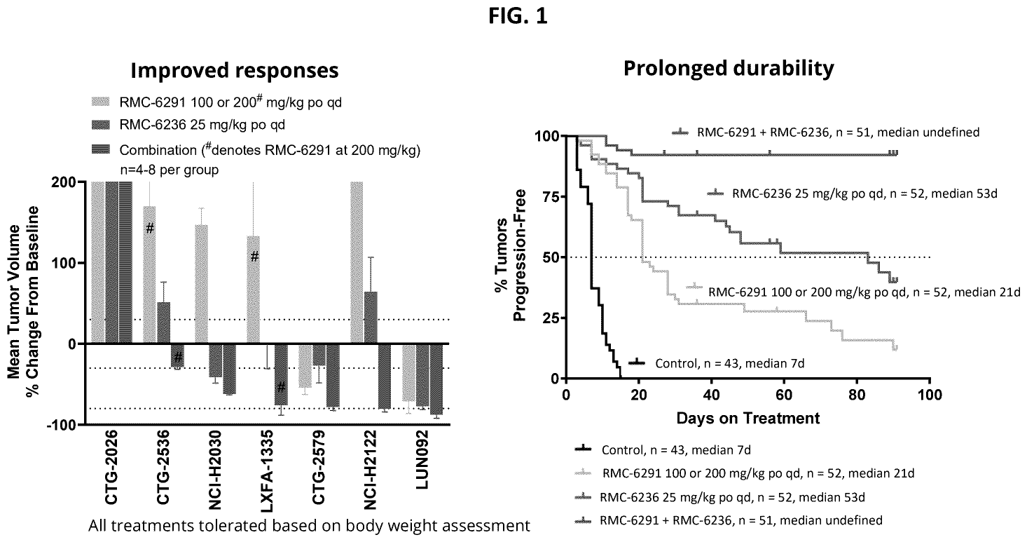

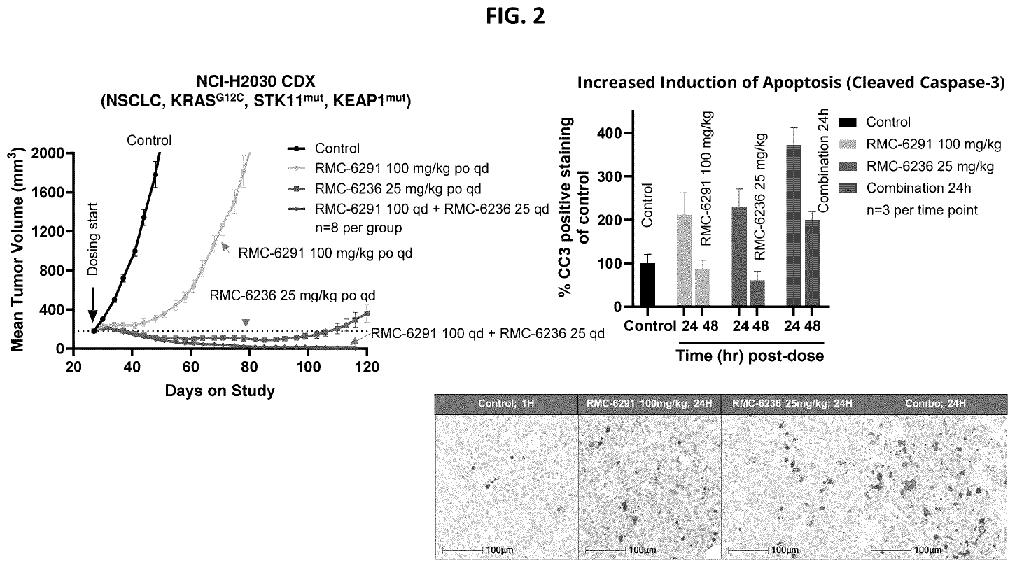

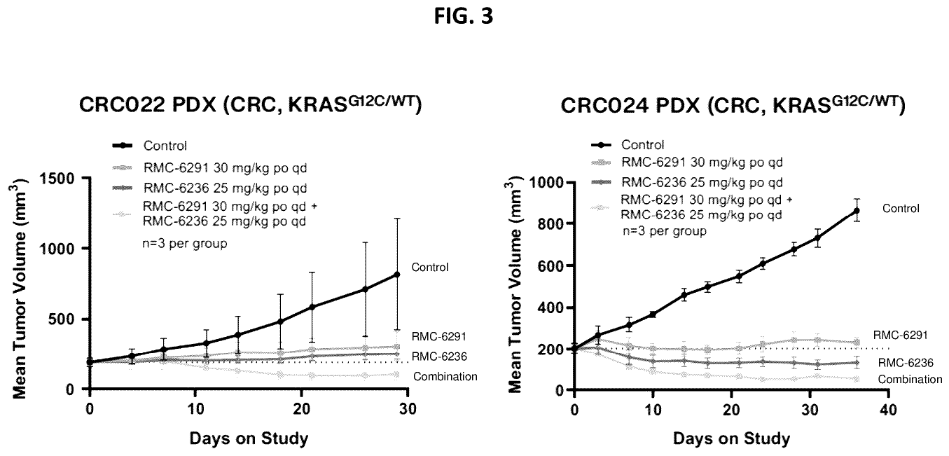

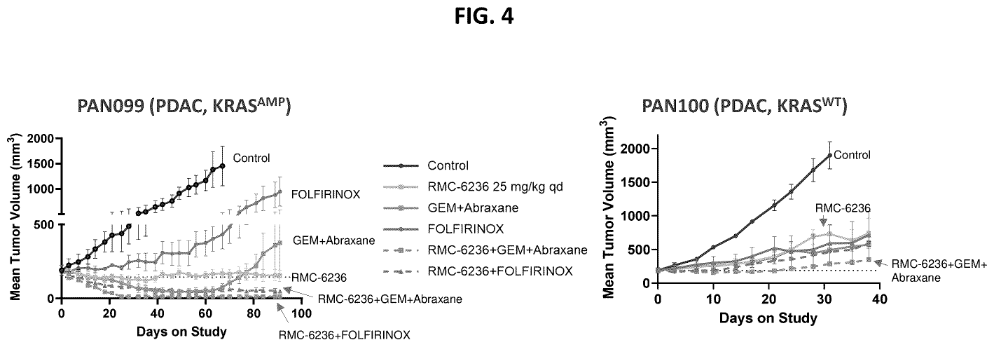

The combination of RMC-6291 and 6236 provides improved responses and prolonged durability in NSCLC models, including difficult to treat co-mutational models (FIG. 1 and FIG. 2). RMC-6291+RMC-6236 demonstrated clear combination benefit in KRAS^{G12C} PDX Models of CRC at clinically translatable doses. The addition of RMC-6236 to RMC-6291 at either 30 mg/kg or 100 mg/kg in these models demonstrated comparable profound combo benefit in depth and durability of response (FIG. 3). RMC-6239 monotherapy shows comparable activity against EGFR-mutant PDAC as chemotherapy and when combined with GEM+Abraxane or FOLFIRINOX, shows a combination benefit resulting in increased activity and durability (FIG. 4).

### Example 2. The RAS(ON) Multi-Selective Inhibitor Blocks Downstream MAPK and PI3K Pathway Activation in KRAS^{G12X}-Mutant Cancers

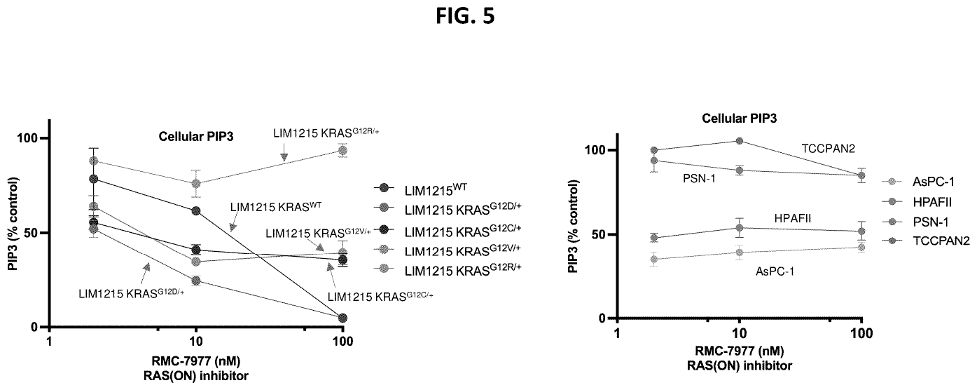

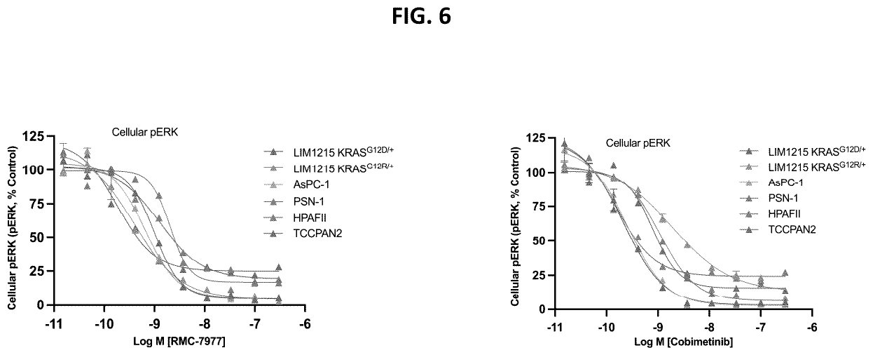

RAS(ON) multi-selective inhibitor RMC-7977 inhibits PI3K activity differently in different RAS mutants. RAS inhibition by RMC-7977 completely suppressed PI3K activity KRAS^{WT} and KRAS^{G12D}, had a moderate inhibitory effect in KRAS^{G12C} and KRAS^{G12V}, and had no effect in KRAS^{G12R} line. RMC-7977 suppresses PIP3 levels only in KRAS^{G12D}-mutant PDAC lines but not in KRAS^{G12R}-mutant PDAC lines (FIG. 5). RAS(ON) multi-selective inhibitor RMC-7977 targets downstream MAPK and PI3K signaling in KRAS^{G12D} cells. RMC-7977 decreased the levels of pERK (MAPK signaling) in both KRAS^{G12D} and KRAS^{G12R} mutant cell lines. MEK inhibitor decreased the levels of pERK (MAPK signaling) in both KRAS^{G12D} and KRAS^{G12R} mutant cell lines (FIG. 6). Direct inhibition of RAS with RAS(ON) multi-selective inhibitor RMC-7977 suppresses both PI3K and MAPK activity in isogenic and mutant KRAS^{G12D} lines, but not KRAS^{G12R} lines, thus providing rationale for a combination of a RAS(ON) multi-selective inhibitor plus a PI3K pathway inhibitor in KRAS^{G12R} mutant cancers.

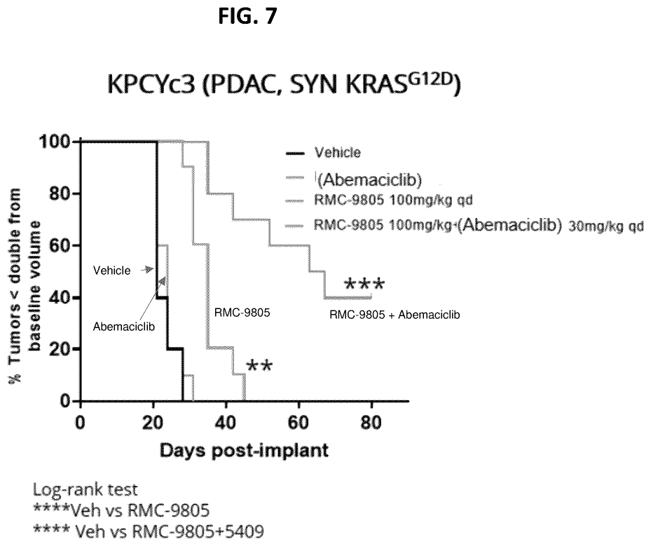

RMC-9805 in combination with Abemaciclib significantly prolongs survival in PDAC models (FIG. 7).

### OTHER EMBODIMENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

1. A method of treating cancer in a subject in need thereof, the method comprising administering to the subject a therapeutically effective amount of a combination, comprising i) a RAS(ON) inhibitor; and ii) one or more additional therapeutic agents.

2. A method of treating a RAS related disease or disorder in a subject in need thereof, the method comprising administering to the subject a therapeutically effective amount of a combination, comprising i) a RAS(ON) inhibitor; and ii) one or more additional therapeutic agents.

3. A method of inhibiting RAS activity and activity of one or more target proteins, in a cell, the method comprising administering to the cell an effective amount of a combination, comprising i) a RAS(ON) inhibitor; and ii) one or more additional therapeutic agents, wherein the one more additional therapeutic agents modulate the activity of the one or more target proteins.

4. The method of any one of claims 1 to 3, wherein the RAS(ON) inhibitor is selected from a RAS(ON) inhibitor described in section I (A).

5. The method of any one of claims 1 to 4, wherein the one or more additional therapeutic agents is a RAS/MAPK pathway inhibitor, a Kinase inhibitor, a Receptor Tyrosine Kinase inhibitor, a PI3K/mTOR pathway inhibitor, a DNA Damage Response inhibitor, a Cell Cycle inhibitor, an Anti-apoptotic protein inhibitor, an Autophagy inhibitor, a Macropinocytosis inhibitor, a Wnt/Beta-catenin pathway inhibitor, a JAK/STAT pathway inhibitor, an Epigenetic modulator, an immunotherapy, a farnesyl transferase inhibitor, a TGFbeta inhibitor, a HSP90 inhibitor, a GPX4 inhibitor, a NRF2 inhibitor, a TEAD inhibitor, a NOTCH inhibitor, a gamma secretase inhibitor, a hedgehog inhibitor, a chemotherapeutic, a proteasome inhibitor, or any combination thereof.

6. The method of claim 5, wherein the RAS/MAPK pathway inhibitor is a RAS (OFF) inhibitor, a SOS1 inhibitor, a SHP2 inhibitor, a MEK inhibitor, a RAF inhibitor, a ERK inhibitor, a MAPK inhibitor, or any combination thereof.

7. The method of claim 6, wherein the RAS (OFF) inhibitor is selected from a RAS (OFF) inhibitor described in section I (b) (i).

8. The method of claim 5, wherein the kinase inhibitor is a PKA inhibitor, a FAK inhibitor, a ROCK inhibitor, a MSK1 inhibitor, a RSK inhibitor, an ALK inhibitor, or any combination thereof.

9. The method of claim 5, wherein the Receptor Tyrosine Kinase inhibitor is an EGFR inhibitor, a HER2 inhibitor, a MET inhibitor, an AXL inhibitor, an IGFR inhibitor, a RET inhibitor, a ROS1 inhibitor, a PDGFR inhibitor, a FGFR inhibitor, a VEGF inhibitor, or any combination thereof.

10. The method of claim 5, wherein the PI3K/mTOR pathway inhibitor is a PI3K inhibitor, an AKT inhibitor, an mTOR inhibitor, an MNK inhibitor, an eIF4 inhibitor, or any combination thereof.

11. The method of claim 10, wherein the eIF4 inhibitor is an eIF4A inhibitor or an eIF4G inhibitor.

12. The method of claim 5, wherein the DNA damage response inhibitor is a Wee1 inhibitor, a CHK inhibitor, an ATM inhibitor, an ATR inhibitor, a PARP inhibitor, a DNA-PK inhibitor, or any combination thereof.

13. The method of claim 5, wherein the cell cycle inhibitor is a CDK inhibitor, an Aurora kinase inhibitor, a PLK inhibitor, a KSP inhibitor, or any combination thereof.

14. The method of claim 13, wherein the CDK inhibitor is a CDK2 inhibitor, a CDK4/6 inhibitor, a CDK7 inhibitor, or a CDK9 inhibitor, or any combination thereof.

15. The method of claim 5, wherein the anti-apoptotic inhibitors is a Bcl inhibitor, an XIAP inhibitor, a survivin inhibitor, an Mcl-1 inhibitor, or a FLIP inhibitor, or any combination thereof.

16. The method of claim 15, wherein the ULK1 inhibitor is a ULK1/2 inhibitor.

17. The method of claim 5, wherein the Wnt/Beta-catenin pathway inhibitor is a beta-catenin inhibitor, a PORCN inhibitor, a GSK3 inhibitor, a CLK inhibitor, or any combination thereof.

18. The method of claim 5, wherein the JAK/STAT pathway inhibitor is an inhibitor of JAK1, JAK2, JAK3, STAT3, STAT5, or any combination thereof.

19. The method of claim 5, wherein the epigenetic modulator is a HDAC inhibitor, a BET inhibitor, an EZH2 inhibitor, a Co-REST inhibitor, an EP300 inhibitor, an LSD1 inhibitor, a PRMT5 inhibitor, an MAT2A inhibitor, a DOTL1 inhibitor, or any combination thereof.

20. The method of claim 5, wherein the immunotherapy is an immune checkpoint inhibitor, a cytokine inhibitor, a cytokine, a vaccine, an antibody therapy, a bispecific antibody, a cellular therapy, or any combination thereof.

21. The method of claim 20, wherein the immunotherapy is described in Table 1.

22. The method of any one of claims 1 to 4, wherein the one or more additional therapeutic agent comprises an immune checkpoint inhibitor and chemotherapeutic agent.

23. The method of any one of claims 1 to 4, wherein the combination comprises an EGFR inhibitor and pembrolizumab.

24. The method of any one of claims 1 to 4, wherein the combination comprises a SHP2 inhibitor, an immune checkpoint inhibitor and CTLA-4 inhibitor.

25. The method of any one of claims 1 to 24, wherein the combination further comprises one or more pharmaceutically acceptable excipients.

26. The method of any one of claims 1 to 25, wherein the cancer is astrocytic, breast, cervical, colorectal, endometrial, esophageal, gastric, head and neck, hepatocellular, laryngeal, lung, oral, ovarian, prostate, and thyroid carcinomas and sarcomas. Other cancers include, for example: Cardiac, for example: sarcoma (angiosarcoma, fibrosarcoma, rhabdomyosarcoma, liposarcoma), myxoma, rhabdomyoma, fibroma, lipoma and teratoma; Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, lipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver, for example: hepatoma (hepatocellular carcinoma), cholangiocarcinoma, hepatoblastoma, angiosarcoma, hepatocellular adenoma, hemangioma; Biliary tract, for example: gall bladder carcinoma, ampullary carcinoma, cholangiocarcinoma; Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors; Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma); Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic, for example: blood (myeloid leukemia (acute and chronic), acute lymphoblastic myeloproliferative neoplasms), multiple myeloma, myelodysplastic syndrome), Hodgkin's disease, non-Hodgkin's lymphoma (malignant lymphoma); Skin, for example: malignant melanoma, basal cell carcinoma, squamous cell carcinoma, Kaposi's sarcoma, moles dysplastic nevi, lipoma, angioma, dermatofibroma, keloids, psoriasis; and Adrenal glands, for example: neuroblastoma.

27. The method of claim 26, wherein the cancer is a lung cancer (e.g., NSCLC) or a gastric cancer (e.g., PDAC, or CRC).

28. The method of any one of claims 1 to 27, wherein the subject has increased progression-free survival relative to a progression-free survival of a subject or subject population receiving a monotherapy with the RAS(ON) inhibitor or a monotherapy of the one or more additional therapeutic agents.

29. The method of any one of claims 1 to 27, wherein the subject has increased probability of a relapse-free survival relative to a probability of a relapse-free survival of a subject or subject population receiving a monotherapy with the RAS(ON) inhibitor or a monotherapy of the one or more additional therapeutic agents.

30. The method of any one of claims 1 to 27, wherein the subject has increased anti-tumor activity relative to an anti-tumor activity in a subject or subject population receiving a monotherapy with the RAS(ON) inhibitor or a monotherapy of the one or more additional therapeutic agents.

31. The method according to any one of claims 1 to 27, wherein the one or more additional therapeutic agents synergistically increases the sensitivity of the cancer cells to the RAS(ON) inhibitor.

32. The method according to any one of claims 1 to 27, wherein the RAS(ON) inhibitor synergistically increases the sensitivity of the cancer cells to the one or more additional therapeutic agents.
